(19) United States
(12) Patent Application Publication
(10) Pub. No.: US 2022/0370509 A1
(43) Pub. Date: Nov. 24, 2022

(54) COMPOSITIONS AND METHODS FOR TREATING OR PREVENTING CROHN'S DISEASE

(71) Applicant: Orchard Therapeutics (Europe) Limited, London (GB)

(72) Inventors: Bobby GASPAR, London (GB); Jia L. WOLFE, Winchester, MA (US); Pervinder SAGOO, St Albans (GB)

(21) Appl. No.: 17/771,524

(22) PCT Filed: Nov. 12, 2020
(86) PCT No.: PCT/US2020/060288

§ 371 Date: Apr. 25, 2022

Related U.S. Application Data
(60) Provisional application No. 62/934,345, filed on Nov. 12, 2019.

(51) Int. Cl. A61K 35/545; A61P 1/02; A61K 45/06; A61K 38/17; A61K 48/00

(52) U.S. Cl. CPC A61K 35/545; A61P 1/02; A61K 45/06; A61K 38/1709; A61K 48/0066

(57) ABSTRACT

Described herein are compositions and methods for treating a subject having or at risk of developing Crohn's disease. Using the compositions and methods of the disclosure, a patient, such as an adult human patient, may be provided one or more agents that elevate the expression and/or activity levels of Nucleotide-binding oligomerization domain-containing protein 2 (NOD2). Exemplary agents that may be used in conjunction with the compositions and methods of the disclosure for this purpose include cells, such as pluripotent cells, that express NOD2.

### FIELD OF THE INVENTION

The disclosure relates to methods for treating Crohn's disease by way of modulating gene expression, as well as compositions that may be used in such methods.

### SEQUENCE LISTING

The instant application contains a Sequence Listing which has been submitted electronically in ASCII format and is hereby incorporated by reference in its entirety. Said ASCII copy, created on Nov. 12, 2020 is named 51139-021WO2_Sequence_Listing_11.12.20_ST25 and is 15,172 bytes in size.

### BACKGROUND

Crohn's disease is an inflammatory bowel disease that causes inflammation of the digestive tract, which can lead to abdominal pain, severe diarrhea, fatigue, weight loss, and malnutrition. There is currently no cure for Crohn's disease, and long-term, effective treatment options are limited. For patients afflicted with Crohn's disease, the disease can have a devastating impact on their lifestyle, as common symptoms of Crohn's disease include diarrhea, cramping, abdominal pain, fever, and even rectal bleeding. Crohn's disease and complications associated with it often result in the patient requiring surgery, often more than once. There remains a need for therapeutic modalities that target underlying causes of Crohn's disease to achieve effective amelioration of symptoms and disease remission.

### SUMMARY OF THE INVENTION

The present disclosure relates to compositions and methods for the treatment of Crohn's disease. In a first aspect, the disclosure provides a method of treating Crohn's disease in a patient (e.g., a mammalian patient, such as a human patient (e.g., an adult human patient)) in need thereof by providing to the patient one or more agents that collectively increase expression and/or activity of functional Nucleotide-binding oligomerization domain-containing protein 2 (NOD2).

In a further aspect, the disclosure provides a method of inducing sustained disease remission of Crohn's disease in a patient (e.g., a mammalian patient, such as a human patient (e.g., an adult human patient)) in need thereof, the method comprising providing to the patient one or more agents that increase expression and/or activity of functional NOD2.

In another aspect, the disclosure provides a method of increasing the sensing of muramyl dipeptide (MDP) by the innate immune system in a patient (e.g., a mammalian patient, such as a human patient (e.g., an adult human patient)) that has Crohn's disease, the method comprising providing to the patient one or more agents that increase expression and/or activity of functional NOD2.

In another aspect, the disclosure provides a method of increasing NFκB signal transduction detection in a patient (e.g., a mammalian patient, such as a human patient (e.g., an adult human patient)) that has Crohn's disease, the method comprising providing to the patient one or more agents that increase expression and/or activity of functional NOD2.

In another aspect, the disclosure provides a method of treating Crohn's disease in a patient (e.g., a mammalian patient, such as a human patient (e.g., an adult human patient)), the method comprising:
(i) determining whether the patient has a loss-of-function mutation in an endogenous gene encoding functional NOD2 (such as R702W, G908R, or L1007fs); and
(ii) administering to the patient one or more agents that increase expression and/or activity of functional NOD2 if the patient has a loss-of-function mutation in the endogenous gene

In some embodiments of any of the foregoing aspects of the disclosure, the patient has a defect in NOD2 expression (e.g., by way of a loss-of-function mutation). They patient may be one, for example, that expresses lower than normal, physiological levels of NOD2.

In some embodiments of any of the foregoing aspects of the disclosure, the patient has a loss-of-function mutation in an endogenous gene encoding functional NOD2. In some embodiments, the loss-of-function mutation in an endogenous gene encoding functional NOD2 is selected from the group consisting of R702W, G908R, and L1007fs.

In another aspect, the disclosure provides one or more agents that increase expression and/or activity of functional NOD2 for use in a method of treating Crohn's disease in a patient (e.g., a mammalian patient, such as a human patient (e.g., an adult human patient)) in need thereof.

In another aspect, the disclosure provides one or more agents that increase expression and/or activity of functional NOD2 for use in therapy.

In another aspect, the disclosure provides one or more agents that increase expression and/or activity of functional NOD2 for use in a method of treating Crohn's disease in a patient (e.g., a mammalian patient, such as a human patient (e.g., an adult human patient)) in need thereof, wherein the method comprises:
(i) determining whether the patient has a loss-of-function mutation in an endogenous gene encoding functional NOD2 (such as R702W, G908R, or L1007fs); and
(ii) administering to the patient one or more agents that increase expression and/or activity of functional NOD2 if the patient has a loss-of-function mutation in the endogenous gene,

In some embodiments of any of the preceding three aspects of the disclosure, the patient has a defect in NOD2 expression (e.g., by way of a loss-of-function mutation). They patient may be one, for example, that expresses lower than normal, physiological levels of NOD2.

In some embodiments of any of the preceding three aspects of the disclosure, the patient has a loss-of-function mutation in an endogenous gene encoding functional NOD2. In some embodiments, the loss-of-function mutation in an endogenous gene encoding functional NOD2 is selected from the group consisting of R702W, G908R, and L1007fs.

In another aspect, the disclosure provides use of one or more agents that increase expression and/or activity of functional NOD2 in the manufacture of a medicament for treating Crohn's disease in a patient (e.g., a mammalian patient, such as a human patient (e.g., an adult human patient)) in need thereof. The patient may have a defect in NOD2 expression (e.g., by way of a loss-of-function mutation). They patient may be one, for example, that expresses lower than normal, physiological levels of NOD2. In some embodiments of this aspect of the disclosure, the patient has a loss-of-function mutation in an endogenous gene encoding functional NOD2. In some embodiments of this aspect of the disclosure, the loss-of-function mutation in an endogenous gene encoding functional NOD2 is selected from the group consisting of R702W, G908R, and L1007fs.

In some embodiments of any of the foregoing aspects of the disclosure, the one or more agents contain (i) one or more nucleic acid molecules that collectively encode NOD2, (ii) one or more interfering RNA molecules that collectively increase expression and/or activity of NOD2, (iii) one or more nucleic acid molecules encoding the one or more interfering RNA molecules (e.g., short interfering RNA (siRNA), short hairpin RNA (shRNA), and/or micro RNA (miRNA)), (iv) one or more of the proteins themselves, and/or (v) one or more small molecules that collectively increase expression and/or activity of NOD2.

### Nucleic Acids Encoding Functional NOD2

In some embodiments, the one or more agents contain a nucleic acid molecule that encodes functional NOD2.

In some embodiments, the nucleic acid molecule that encodes functional NOD2 has at least 85% sequence identity (e.g., 85%, 86%, 87%, 88%, 89%, 90%, 91%, 92%, 93%, 94%, 95%, 96%, 97%, 98%, 99%, or 100% sequence identity) to the nucleic acid sequence of SEQ ID NO: 1. In some embodiments, the nucleic acid has at least 90% sequence identity (e.g., 90%, 91%, 92%, 93%, 94%, 95%, 96%, 97%, 98%, 99%, or 100% sequence identity) to the nucleic acid sequence of SEQ ID NO: 1. In some embodiments, the nucleic acid has at least 95% sequence identity (e.g., 95%, 96%, 97%, 98%, 99%, or 100% sequence identity) to the nucleic acid sequence of SEQ ID NO: 1. In some embodiments, the nucleic acid has the nucleic acid sequence of SEQ ID NO: 1.

In some embodiments, the encoded functional NOD2 protein has an amino acid sequence that is at least 85% identical (e.g., 85%, 86%, 87%, 88%, 89%, 90%, 91%, 92%, 93%, 94%, 95%, 96%, 97%, 98%, 99%, or 100% identical) to the amino acid sequence of SEQ ID NO: 2. In some embodiments, the encoded functional NOD2 protein has an amino acid sequence that is at least 90% identical (e.g., 90%, 91%, 92%, 93%, 94%, 95%, 96%, 97%, 98%, 99%, or 100% identical) to the amino acid sequence of SEQ ID NO: 2. In some embodiments, the encoded functional NOD2 protein has an amino acid sequence that is at least 95% identical (e.g., 95%, 96%, 97%, 98%, 99%, or 100% identical) to the amino acid sequence of SEQ ID NO: 2. In some embodiments, the encoded functional NOD2 protein has the amino acid sequence of SEQ ID NO: 2.

In some embodiments, the functional NOD2 protein has an amino acid sequence that differs from the amino acid sequence of SEQ ID NO: 2 by way of one or more conservative amino acid substitutions (e.g., by way of from 1 to 50 conservative amino acid substitutions).

### Nucleic Acids Provided to the Patient by Cellular Therapy

In some embodiments, the nucleic acid molecule is provided to the patient by administering to the patient a composition containing a population of cells that express functional NOD2. The cells may be pluripotent cells, such as CD34+ cells (e.g., hematopoietic stem cells and/or myeloid progenitor cells), induced pluripotent stem cells, and/or embryonic stem cells.

In some embodiments, the composition is administered systemically to the patient. For example, the composition may be administered to the patient by way of intravenous injection.

In some embodiments, the cells are autologous cells with respect to the patient. In some embodiments, the cells are allogeneic cells with respect to the patient (e.g., HLA-matched allogeneic cells).

In some embodiments, the cells (e.g., pluripotent cells, such as CD34+ cells (e.g., hematopoietic stem cells or myeloid progenitor cells), induced pluripotent stem cells, and/or embryonic stem cells) are transduced ex vivo to express functional NOD2. For example, the cells may be transduced with a viral vector selected from the group consisting of an adenovirus, a parvovirus, a coronavirus, a rhabdovirus, a paramyxovirus, a picornavirus, an alphavirus, a herpes virus, a poxvirus, and a Retroviridae family virus. In some embodiments, the viral vector is a Retroviridae family viral vector, such as a lentiviral vector, alpharetroviral vector, or gammaretroviral vector. In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector is a pseudotyped viral vector, such as a pseudotyped viral vector selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the cells are transduced by contacting the cells with a viral vector (e.g., a viral vector described above) and a substance that reduces activity and/or expression of protein kinase C (PKC).

In some embodiments, the substance that reduces activity and/or expression of PKC is a PKC inhibitor. The PKC inhibitor may be staurosporine or a variant thereof.

In some embodiments, the cell is further contacted with stauprimide, e.g., as described in Caravatti et al. Bioorg. Med. Chem. Letters 4:199-404, 1994, the disclosure of which is hereby incorporated by reference in its entirety.

In some embodiments, the method of transducing the cells comprises contacting the cell with a histone deacetylase (HDAC) inhibitor.

In some embodiments, the method of transducing the cells comprises contacting the cells with an activator of prostaglandin E receptor signaling.

In some embodiments, the activator of prostaglandin E receptor signaling is a small molecule, such as a compound described in WO 2007/112084 or WO 2010/108028, the disclosures of each of which are incorporated herein by reference as they pertain to prostaglandin E receptor signaling activators.

In some embodiments, the activator of prostaglandin E receptor signaling is a small molecule, such as a small organic molecule, a prostaglandin, a Wnt pathway agonist, a cAMP/PI3K/AKT pathway agonist, a Ca²⁺ second messenger pathway agonist, a nitric oxide (NO)/angiotensin signaling agonist, or another compound known to stimulate the prostaglandin signaling pathway, such as a compound selected from Mebeverine, Flurandrenolide, Atenolol, Pindolol, Gaboxadol, Kynurenic Acid, Hydralazine, Thiabendazole, Bicuclline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and or a derivative of any of these compounds.

In some embodiments, the activator of prostaglandin E receptor signaling is a naturally-occurring or synthetic chemical molecule or polypeptide that binds to and/or interacts with a prostaglandin E receptor, typically to activate or increase one or more of the downstream signaling pathways associated with a prostaglandin E receptor.

In some embodiments, the activator of prostaglandin E receptor signaling is selected from the group consisting of prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling is PGE2 or dmPG2.

In some embodiments, the activator of prostaglandin E receptor signaling is 15d-PGJ2, delta12-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, I I-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxy PGE2.

In some embodiments, the activator of prostaglandin E receptor signaling is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling is a non-PGE2-based ligand. In some embodiments, the activator of prostaglandin E receptor signaling is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE2-based EP4 agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists

In some embodiments, the activator of prostaglandin E receptor signaling is a Wnt agonist. Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt1Oa, Wnt1Ob, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃ or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, Calif.)). The structure of CHIR-99021 is

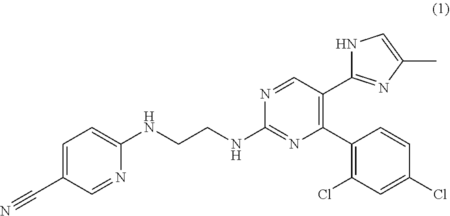 [SMILES: Cc1c[nH]c(-c2cnc(NCCNc3ccc(C#N)cn3)nc2-c2ccc(Cl)cc2Cl)n1]

or a salt thereof.

The structure of CHIR-98023 is

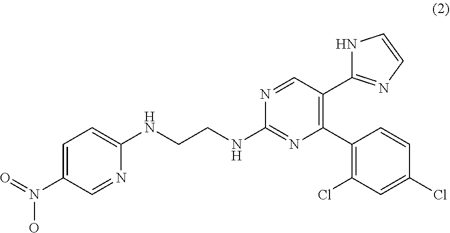

or a salt thereof.

In some embodiments, the activator of prostaglandin E receptor signaling is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling is an agent that increases signaling through the NO/Angiotensin signaling, such as an agent selected from the group consisting of L-Arg, Sodium Nitroprusside, Sodium Vanadate, Bradykinin, and derivatives thereof.

In some embodiments, the method of transducing the cells comprises contacting the cells with a GSK3 inhibitor.

In some embodiments, the GSK3 inhibitor is CHIR-99021 or CHIR-98023.

In some embodiments, the GSK3 inhibitor is Li₂CO₃.

In some embodiments, the method of transducing the cells comprises contacting the cells with caraphenol A, which is described, for example, in Ozog et al., Blood 134:1298-1311 (2019), the disclosure of which is incorporated herein by reference.

In some embodiments, the method of transducing the cells comprises contacting the cells with a polycationic polymer.

In some embodiments, the polycationic polymer is polybrene, protamine sulfate, polyethylenimine, or a polyethylene glycol/poly-L-lysine block copolymer.

In some embodiments, the polycationic polymer is protamine sulfate.

In some embodiments, the method of transducing the cells comprises contacting the cells with an agent that inhibits mTOR signaling. The agent that inhibits mTOR signaling may be, for example, rapamycin, among other suppressors of mTOR signaling.

In some embodiments, the method of transducing the cells comprises contacting the cells with tacrolimus and/or vectorfusin.

In some embodiments, the method of transducing the cells comprises contacting the cells with a cyclosporine, such as cyclosporine A (CsA) or cyclosporine H (CsH).

In some embodiments, the cells are spun (e.g., by centrifugation, i.e., “centrifuged”) while being contacted with the viral vector (e.g., in combination with the one or more agents described above). This process, referred to herein as “spinoculation,” may occur with a centripetal force of, e.g., from about 200×g to about 2,000×g. In some embodiments, the cells are spun at a centripetal force of from about 300× g to about 1,200×g while being contacted with the viral vector (e.g., in combination with the one or more agents described above). For example, the cells may be spun at a centripetal force of about 300× g, 400× g, 500× g, 600× g, 700× g, 800× g, 900× g, 1,000×g, 1,100×g, or 1,200×g while being contacted with the viral vector (e.g., in combination with the one or more agents described above). In some embodiments, the cells are spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the cells are spun at room temperature, such as at a temperature of about 25° C.

In some embodiments, the cells (e.g., pluripotent cells, such as CD34+ cells (e.g., hematopoietic stem cells or myeloid progenitor cells), induced pluripotent stem cells, and/or embryonic stem cells) are transfected ex vivo to express the one or more proteins. For example, the cells may be transfected using an agent selected from the group consisting of a cationic polymer, diethylaminoethyldextran, polyethylenimine, a cationic lipid, a liposome, calcium phosphate, an activated dendrimer, and a magnetic bead. In some embodiments, the cells are transfected using a technique selected from the group consisting of electroporation, Nucleofection, squeeze-poration, sonoporation, optical transfection, Magnetofection, and impalefection.

In some embodiments, the cells (e.g., pluripotent cells, such as CD34+ cells (e.g., hematopoietic stem cells or myeloid progenitor cells), induced pluripotent stem cells, and/or embryonic stem cells) are obtained by recapitulating a functional NOD2 gene in the cells at a genetic locus that encodes a defective NOD2 protein, for example, in circumstances in which the cells are autologous cells obtained from the patient suffering from Crohn's disease. For example, in some embodiments, the cells are obtained by delivering to the cells, ex vivo, a nuclease that catalyzes cleavage of a phosphodiester bond at a target position within the genome of the cell, such as a position encoding endogenous NOD2.

The nuclease may be, for example, a clustered regularly interspaced short palindromic repeats (CRISPR)-associated protein. In some embodiments, the CRISPR-associated protein is CRISPR-associated protein 9 (Cas9) or CRISPR-associated protein 12a (Cas12a). In some embodiments, the CRISPR-associated protein is Cas1, Cas1B, Cas2, Cas3, Cas4, Cas5, Cas6, Cas7, Cas8, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, or Cpf1 endonuclease, or a homolog thereof, a recombination of the naturally occurring molecule thereof, optionally expressed using a codon-optimized gene thereof.

When CRISPR-associated proteins are used, in addition to delivery of the nuclease to the cell, the cell may be delivered a guide ribonucleic acid (gRNA). The gRNA may direct the nuclease to site-specifically effectuate one or more single-strand breaks or double-strand breaks within or near an endogenous region of the cell genome that encodes NOD2. For example, the gRNA may direct the nuclease to effectuate one or more single-strand breaks or double-strand breaks within or near an endogenous NOD2 gene in the cell genome. In such instances, the nuclease may induce a permanent deletion or inactivation of the endogenous NOD2 gene in the cell genome.

In some embodiments, the gRNA may direct the nuclease to effectuate one or more single-strand breaks or double-strand breaks within or near a region of the cell genome that contains a transcription regulatory element that controls NOD2 expression, such as a region of the cell genome that contains an endogenous NOD2 promoter. In such instances, the nuclease may induce a permanent deletion or inactivation of the transcription regulatory element that controls NOD2 expression, such as a permanent deletion of inactivation of an endogenous NOD2 promoter.

In addition to delivery of a nuclease and gRNA, the cell may be delivered a nucleic acid template containing a functional NOD2 gene. In such instances, the nuclease may effectuate a single-strand break or double-strand break at a locus within or near the endogenous NOD2 gene in the cell, facilitating insertion of the nucleic acid template containing a functional NOD2 gene into the locus containing the single-strand break or double-strand break. This insertion results in a permanent insertion or inactivation of the endogenous NOD2 gene and stable expression of the functional NOD2 gene encoded by the template nucleic acid.

In some embodiments, in lieu of delivering to the cells a template nucleic acid encoding functional NOD2, the nuclease that is delivered to the cells may be used to directly edit the cell genome so as to negate a defect-causing NOD2 mutation and recapitulate functional NOD2 gene expression. For example, in a technique known as base-editing, the nuclease may be catalytically impaired with respect to nuclease activity, and may be fused to a reverse transcriptase that itself contains the gRNA. The nuclease is unable to catalyze single-strand or double-strand breaks, but retains the helicase activity required to unwind the target DNA. The reverse transcriptase may then use the gRNA to direct the reverse transcriptase to a target site within the cell genome that contains, for example, a mutation causing a defect in NOD2 activity. Preferably, the gRNA encodes an edit to the genetic locus (e.g., encodes an amino acid substitution) that restores functional NOD2 activity and negates the defect-causing mutation. Once localized to the target site, the gRNA (termed a “prime-editing guide RNA” or “pegRNA”) may then serve as a template from which the reverse transcriptase may effectuate the desired nucleotide edit. Methods for using pegRNA-containing reverse transcriptases to site-specifically edit genetic loci are described, for example, in Anzalone et al., Nature (2019) doi:10.1038/s41586-019-1711-4, the disclosure of which is incorporated herein by reference. Additional methods for DNA base editing that may be used to negate a defect-causing NOD2 mutation in the cells and recapitulate expression of a functional NOD2 protein are described in Cohen, “Novel CRISPR-derived ‘base editors’ surgically alter DNA or RNA, offering new ways to fix mutations,’ Science Magazine, October 2017, the disclosure of which is incorporated herein by reference.

In some embodiments, the nuclease delivered to the cells is a transcription activator-like effector nuclease, a meganuclease, or a zinc finger nuclease. In these instances, as well, the cells may additionally be contacted with a nucleic acid molecule encoding functional NOD2 while the cells are contacted with the nuclease. In some embodiments, the nucleic acid molecule encoding functional NOD2 comprises a 5′ homology arm and a 3′ homology arm having nucleic acid sequences that are sufficiently similar to the nucleic acid sequences located 5′ to the target position and 3′ to the target position, respectively, to promote homologous recombination.

In some embodiments, the nuclease, gRNA, and/or template nucleic acid described above, are delivered to the cells by contacting the cells with a viral vector that encodes the nuclease, gRNA, and/or template nucleic acid. For example, the viral vector that encodes the nuclease, gRNA, and/or template nucleic acid may be an adeno-associated virus (AAV), an adenovirus, a parvovirus, a coronavirus, a rhabdovirus, a paramyxovirus, a picornavirus, an alphavirus, a herpes virus, a poxvirus, and a Retroviridae family virus. In some embodiments, the viral vector that encodes the nuclease, gRNA, and/or template nucleic acid is a Retroviridae family viral vector, such as a lentiviral vector, alpharetroviral vector, or gammaretroviral vector. In some embodiments, the Retroviridae family viral vector that encodes the nuclease, gRNA, and/or template nucleic acid contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector that encodes the nuclease, gRNA, and/or template nucleic acid is an integration-deficient lentiviral vector (IDLV), as described, for example, in Wanisch et al., Mol Ther. 17:1316-1332 (2009), the disclosure of which is incorporated herein by reference. In some embodiments, the viral vector that encodes the nuclease, gRNA, and/or template nucleic acid is an AAV selected from the group consisting of AAV1, AAV2, AAV3, AAV4, AAV5, AAV6, AAV7, AAV8, AAV9, AAV10, and AAVrh74. In some embodiments, the viral vector that encodes the nuclease, gRNA, and/or template nucleic acid is a pseudotyped viral vector, such as a pseudotyped viral vector selected from the group consisting of a pseudotyped AAV, a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the nuclease, gRNA, and/or template nucleic acid is delivered to the cells by transfecting the cells with a gene that encodes the nuclease and/or gRNA, and/or that contains the template nucleic acid. For example, the cells may be transfected with a gene that encodes the nuclease and/or gRNA, and/or that contains the template nucleic acid, by contacting the cells with a gene encoding the nuclease and/or gRNA, and/or that contains the template nucleic acid, in the presence of a cationic polymer, diethylaminoethyldextran, polyethylenimine, a cationic lipid, a liposome, calcium phosphate, an activated dendrimer, and/or a magnetic bead. In some embodiments, the cells are transfected with a gene that encodes the nuclease and/or gRNA, and/or that contains the template nucleic acid, using a technique selected from the group consisting of electroporation, Nucleofection, squeeze-poration, sonoporation, optical transfection, Magnetofection, and impalefection.

Additional methods that may be used to deliver the nuclease, gRNA, and/or template nucleic acid to the cells are described, for example, in Martin et al., Cell Stem Cell 24:821-828 (2019), the disclosure of which is incorporated herein by reference.

In some embodiments, prior to administering the composition to the patient, a population of precursor cells is isolated from the patient or a donor, and the precursor cells are expanded ex vivo to yield the population of cells being administered to the patient. In some embodiments, the precursor cells are CD34+ HSCs. In some embodiments, the precursor cells are expanded without loss of HSC functional potential.

In some embodiments, the precursor cells are expanded ex vivo by contacting the precursor cells with one or more cell expansion agents described herein or known in the art to promote cell proliferation, thereby yielding the population of cells being administered to the subject. For example, the expansion agent may be StemRegenin 1, also known in the art as compound SR1, represented by formula (3), below.

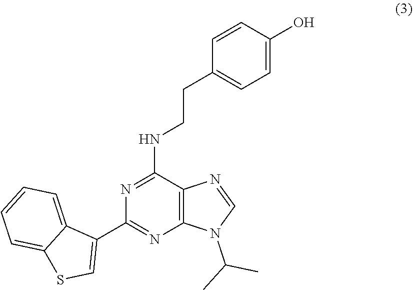 [SMILES: CC(C)n1cnc2c(NCCc3ccc(O)cc3)nc(-c3csc4ccccc34)nc21]

SR1 and other expansion agents are described, for example, in U.S. Pat. Nos. 8,927,281 and 9,580,426, the disclosures of each of which are incorporated herein by reference in their entirety.

Additional expansion agents that may be used in conjunction with the compositions and methods of the disclosure include compound UM-171, which is described in U.S. Pat. No. 9,409,906, the disclosure of which is incorporated herein by reference in its entirety. Expansion agents that may be used herein further include structural or stereoisomeric variants of compound UM-171, such as the compounds described in US 2017/0037047, the disclosure of which is incorporated herein by reference in its entirety. The structure of compound UM-171 is shown in formula (4), below.

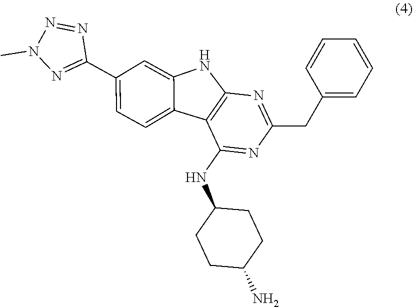 [SMILES: Cn1nnc(-c2ccc3c(c2)[nH]c2nc(Cc4ccccc4)nc(N[C@H]4CC[C@H](N)CC4)c23)n1]

In some embodiments, the expansion agent is a bromide salt of compound (5), such as a compound represented by formula (5), below.

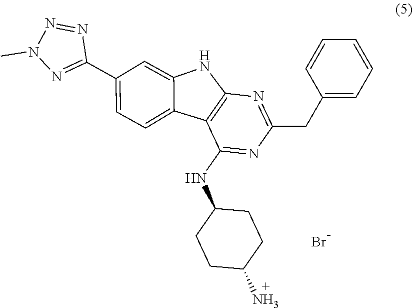 [SMILES: Cn1nnc(-c2ccc3c(c2)[nH]c2nc(Cc4ccccc4)nc(N[C@H]4CC[C@H](N)CC4)c23)n1 ; Br]

Additional expansion agents that may be used in conjunction with the compositions and methods of the disclosure include histone deacetylase (HDAC) inhibitors, as described, for example, in WO 2000/023567, the disclosure of which is incorporated herein by reference. Exemplary agents that may be used to expand a population of precursor cells as described herein are trichostatin A, trapoxin, trapoxin A, chlamydocin, sodium butyrate, dimethyl sulfoxide, suberanilohydroxamic acid, m-carboxycinnamic acid bishydroxamide, HC-toxin, Cyl-2, WF-3161, depudecin, and radicicol, among others.

In some embodiments, prior to isolation of the precursor cells from the patient or donor, the patient or donor is administered one or more pluripotent cell mobilization agents that stimulate the migration of pluripotent cells (e.g., CD34+ HSCs and HPCs) from a stem cell niche, such as the bone marrow, to peripheral circulation. Exemplary cell mobilization agents that may be used in conjunction with the compositions and methods of the disclosure are described herein and known in the art. For example, the mobilization agent may be a C—X—C motif chemokine receptor (CXCR) 2 (CXCR2) agonist. The CXCR2 agonist may be Gro-beta, or a truncated variant thereof. Gro-beta and variants thereof are described, for example, in U.S. Pat. Nos. 6,080,398; 6,447,766; and 6,399,053, the disclosures of each of which are incorporated herein by reference in their entirety. Additionally or alternatively, the mobilization agent may include a CXCR4 antagonist, such as plerixafor or a variant thereof. Plerixafor and structurally similar compounds are described, for example, in U.S. Pat. Nos. 6,987,102; 7,935,692; and 7,897,590, the disclosures of each of which are incorporated herein by reference. Additionally or alternatively, the mobilization agent may include granulocyte colony-stimulating factor (G-CSF). The use of G-CSF as an agent to induce mobilization of pluripotent cells (e.g., CD34+ HSCs and/or HPCs) from a stem cell niche to peripheral circulation is described, for example, in US 2010/0178271, the disclosure of which is incorporated herein by reference in its entirety.

In some embodiments, prior to administering the population of cells to the patient, a population of endogenous pluripotent cells (e.g., a population of endogenous CD34+ HSCs or HPCs) is ablated in the patient by administration of one or more conditioning agents to the subject. In some embodiments, the method includes ablating a population of endogenous pluripotent cells (e.g., a population of endogenous CD34+ HSCs or HPCs) in the patient by administering to the subject one or more conditioning agents prior to administering to the subject the population of cells. The one or more conditioning agents may be myeloablative conditioning agents that deplete a wide variety of hematopoietic cells from the bone marrow of the subject. For example, the one or more conditioning agents may include an alkylating agent, such as a nitrogen mustard (e.g., bendamustine, chlorambucil, cyclophosphamide, ifosfamide, mechlorethamine, or melphalan), a nitrosourea (e.g., carmustine, lomustine, or streptozocin), an alkyl sulfonate (e.g., busulfan), a triazine (e.g., dacarbazine or temozolomide), or an ethylenimine (e.g., altretamine or thiotepa). In some embodiments, the one or more conditioning agents are non-myeloablative conditioning agents that selectively target and ablate a specific population of endogenous pluripotent cells, such as a population of endogenous CD34+ HSCs or HPCs. For example, the one or more conditioning agents may include cytarabine, antithymocyte globulin, fludarabine, or idarubicin.

In some embodiments, upon administration of the population of cells to the subject, the administered cells, or progeny thereof, differentiate into one or more cell types selected from megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

### Nucleic Acids Provided to the Patient by Viral Gene Therapy

In some embodiments, the nucleic acid molecule that encodes functional NOD2 is provided to the patient by administering to the patient one or more viral vectors that contain the nucleic acid molecule encoding functional NOD2. In some embodiments, the one or more viral vectors are administered systemically to the patient. For example, the one or more viral vectors may be administered to the patient by way of intravenous injection. In some embodiments, the one or more viral vectors contain an adenovirus, a parvovirus, a coronavirus, a rhabdovirus, a paramyxovirus, a picornavirus, an alphavirus, a herpes virus, a poxvirus, or a Retroviridae family virus. In some embodiments, the viral vector is a Retroviridae family viral vector, such as a lentiviral vector, alpharetroviral vector, or gammaretroviral vector. In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector is a pseudotyped viral vector, such as a pseudotyped viral vector selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

### Transcription Regulatory Elements

In some embodiments of any of the foregoing aspects or embodiments of the disclosure, the nucleic acid molecule contains a transgene encoding functional NOD2 operably linked to a ubiquitous promoter. The ubiquitous promoter may be, for example, an elongation factor 1-alpha (EF1α) promoter or an intron-less form of the EF1α promoter known as the EF1α short (EFS) promoter. In some embodiments, the nucleic acid molecule contains a transgene encoding functional NOD2 operably linked to a tissue-specific promoter. The tissue-specific promoter may be, for example, a sp146/p47 promoter, CD11b promoter, CD68 promoter, sp146/gp9 promoter, or an endogenous NOD2 promoter.

### Patient Populations

In some embodiments of any of the foregoing aspects or embodiments of the disclosure, the patient is a mammal, such as a human. In some embodiments, the patient has a loss-of-function mutation in an endogenous gene encoding functional NOD2. The mutation may be, for example, R702W, G908R, or L1007fs. In some embodiments, the mutation is heterozygous. In some embodiments, the mutation is homozygous.

In some embodiments, the patient has previously been treated with one or more immunosuppressive agents, biologic agents, and/or corticosteroids. In some embodiments, the patient has not responded to treatment with the one or more immunosuppressive agents, biologic agents, and/or corticosteroids. The one or more immunosuppressive agents may comprise, for example, azathioprine, methotrexate and/or infliximab or other biological agents, including other monoclonal antibodies.

In some embodiments, prior to providing the patient with the one or more agents that increase expression and/or activity of functional NOD2, the patient exhibits persistent disease activity, as assessed by endoscopy, colonoscopy, and/or magnetic resonance enterography.

In some embodiments, prior to providing the patient with the one or more agents that increase expression and/or activity of functional NOD2, the patient has been determined to be at risk of short bowel disease and/or refractory colonic disease if the patient were to undergo an imminent surgical procedure.

In some embodiments, prior to providing the patient with the one or more agents that increase expression and/or activity of functional NOD2, the patient exhibits a persistent perianal lesion such that the patient is not a candidate for coloproctectomy.

In some embodiments, prior to providing the patient with the one or more agents that increase expression and/or activity of functional NOD2, the patient exhibits impaired function and/or quality of life. The function and/or quality of life may be assessed, for example, by way of an Inflammatory Bowel Disease Questionnaire (IBDQ), a European Questionnaire of Life Quality, or a Karnofsky Index.

Therapeutic Activities of Agents that Increase NOD2 Expression

In some embodiments, after providing the patient with the one or more agents that increase expression and/or activity of functional NOD2, the patient exhibits sustained disease remission. For example, the patient may exhibit sustained disease remission for 30 days, two months, three months, four months, five months, six months, seven months, eight months, nine months, ten months, eleven months, or one year or longer after providing the patient with the one or more agents that increase expression and/or activity of functional NOD2.

In some embodiments, after providing the patient with the one or more agents that increase expression and/or activity of functional NOD2, the patient no longer requires treatment with immunosuppressive agents, biologic agents, and/or corticosteroids. For example, the patient may not require treatment with the immunosuppressive agents, biologic agents, and/or corticosteroids for at least three months (e.g., for from about three months to about one year, such as for three months, four months, five months, six months, seven months, eight months, nine months, ten months, eleven months, or one year, or longer). In some embodiments, the patient does not require treatment with the immunosuppressive agents, biologic agents, and/or corticosteroids for up to five years.

In some embodiments, after providing the patient with the one or more agents that increase expression and/or activity of functional NOD2, the patient does not exhibit evidence of erosive disease in the gastrointestinal tract, as assessed by endoscopy and/or radiology.

For example, after providing the patient with the one or more agents that increase expression and/or activity of functional NOD2, the patient may not exhibit evidence of erosive disease in the gastrointestinal tract, as assessed by endoscopy and/or radiology for at least three months (e.g., for from about three months to about one year, such as for three months, four months, five months, six months, seven months, eight months, nine months, ten months, eleven months, or one year, or longer). In some embodiments, the patient does not exhibit evidence of erosive disease in the gastrointestinal tract, as assessed by endoscopy and/or radiology for up to five years.

### Pharmaceutical Compositions—Cells Expressing Functional NOD2

In a further aspect, the disclosure provides a pharmaceutical composition comprising (i) a population of cells (e.g., pluripotent cells) that express functional NOD2 and (ii) one or more carriers, diluents, and/or excipients. The cells (e.g., pluripotent cells) may be, for example, human cells, such as human HSCs or HPCs. In some embodiments, the cells are embryonic stem cells. In some embodiments, the cells are induced pluripotent stem cells. The cells may be, for example, CD34+ cells, such as myeloid progenitor cells. In some embodiments, the cells are CD34+, CD38−, CD45RA−, CD90+, CD49F+, and/or lin−.

In some embodiments, the composition is formulated for administration to a human patient. For example, the composition may be formulated for intravenous injection to the human patient.

In some embodiments, the cells (e.g., pluripotent cells) are autologous with respect to the patient. In some embodiments, the cells (e.g., pluripotent cells) are allogeneic with respect to the patient (e.g., HLA-matched with respect to the patient).

In some embodiments, the cells (e.g., pluripotent cells) comprise a transgene encoding functional NOD2 operably linked to a ubiquitous promoter. The promoter may be an EF1α promoter or an EFS promoter. The cells may comprise a transgene encoding functional NOD2 operably linked to a tissue-specific promoter. The promoter may be, for example, a CD11b promoter, sp146/p47 promoter, CD68 promoter, sp146/gp9 promoter, or an endogenous NOD2 promoter.

### Pharmaceutical Compositions—Viral Vectors Expressing Functional NOD2

In another aspect, the disclosure provides a pharmaceutical composition comprising (i) a viral vector that encodes functional NOD2 and (ii) one or more carriers, diluents, and/or excipients.

In some embodiments, the viral vector is selected from the group consisting of a Retroviridae family virus, an adenovirus, a parvovirus, a coronavirus, a rhabdovirus, a paramyxovirus, a picornavirus, an alphavirus, a herpes virus, and a poxvirus.

In some embodiments, the viral vector is a Retroviridae family viral vector, such as a lentiviral vector, alpharetroviral vector, or gammaretroviral vector. In some embodiments, the Retroviridae family viral vector contains a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR. In some embodiments, the viral vector is a pseudotyped viral vector, such as a pseudotyped viral vector selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

In some embodiments, the composition is formulated for administration to a human patient, for example, by way of intravenous injection to the patient.

In some embodiments, the viral vector comprises a transgene encoding functional NOD2 operably linked to a ubiquitous promoter. The promoter may be an EF1α promoter or an EFS promoter. In some embodiments, the viral vector comprises a transgene encoding NOD2 operably linked to a tissue-specific promoter. In some embodiments, the promoter is a CD11b promoter, sp146/p47 promoter, CD68 promoter, sp146/gp9 promoter, or an endogenous NOD2 promoter.

### Kits

In a further aspect, the disclosure provides a kit comprising the pharmaceutical composition of any one of the foregoing aspects or embodiments of the disclosure. The kit may further comprise a package insert instructing a user of the kit to administer the pharmaceutical composition to a human patient having Crohn's disease. In some embodiments, the package insert instructs a user of the kit to perform the method of the above aspects or embodiments of the disclosure.

### Definitions

As used herein, the terms “ablate,” “ablating,” “ablation,” “condition,” “conditioning,” and the like refer to the depletion of one or more cells in a population of cells in vivo or ex vivo. In some embodiments of the present disclosure, it may be desirable to ablate endogenous cells within a patient (e.g., a patient undergoing treatment for a disease described herein) before administering a therapeutic composition, such as a therapeutic population of cells, to the patient. This can be beneficial, for example, in order to provide newly-administered cells with an environment within which the cells may engraft. Ablation of a population of endogenous cells can be performed in a manner that selectively targets a specific cell type, for example, using antibodies or antibody-drug conjugates that bind to an antigen expressed on the target cell and subsequently engender the killing of the target cell. Additionally or alternatively, ablation may be performed in a non-specific manner using cytotoxins that do not localize to a particular cell type, but are instead capable of exerting their cytotoxic effects on a variety of different cells. Examples of ablation include depletion of at least 5% of cells (e.g., at least 5%, 10%, 15%, 20%, 25%, 30%, 35%, 40%, 45%, 50%, or more) in a population of cells in vivo or in vitro. Quantifying cell counts within a sample of cells can be performed using a variety of cell-counting techniques, such as through the use of a counting chamber, a Coulter counter, flow cytometry, or other cell-counting methods known in the art.

Exemplary agents that can be used to “ablate” a population of cells in a patient (i.e., to “condition”) a patient for treatment) in accordance with the compositions and methods of the disclosure include alkylating agents, such as nitrogen mustards (e.g., bendamustine, chlorambucil, cyclophosphamide, ifosfamide, mechlorethamine, or melphalan), nitrosoureas (e.g., carmustine, lomustine, or streptozocin), alkyl sulfonates (e.g., busulfan), triazines (e.g., dacarbazine or temozolomide), or ethylenimines (e.g., altretamine or thiotepa). In some embodiments, the one or more conditioning agents are non-myeloablative conditioning agents that selectively target and ablate a specific population of endogenous pluripotent cells, such as a population of endogenous CD34+ HSCs or HPCs. For example, the one or more conditioning agents may include cytarabine, antithymocyte globulin, fludarabine, or idarubicin.

As used herein, the term “about” refers to a quantity that varies by as much as 30% (e.g., 25%, 20%, 25%, 10%, 9%, 8%, 7%, 6%, 5%, 4%, 3%, 2% or 1%) relative to a reference quantity.

As used herein in the context of a protein of interest, the term “activity” refers to the biological functionality that is associated with a wild-type form of the protein. For example, in the context of an enzyme, the term “activity” refers to the ability of the protein to effectuate substrate turnover in a manner that yields the product of a corresponding chemical reaction. Activity levels of enzymes can be detected and quantitated, for example, using substrate turnover assays known in the art. As another example, in the context of a membrane-bound receptor, the term “activity” may refer to signal transduction initiated by the receptor, e.g., upon binding to its cognate ligand. Activity levels of receptors involved in signal transduction pathways can be detected and quantitated, for example, by observing an increase in the outcome of receptor signaling, such as an increase in the transcription of one or more genes (which may be detected, e.g., using polymerase chain reaction techniques known in the art).

As used herein, a compound that “activates prostaglandin E receptor signaling” or the like refers to a compound having the ability to increase signal transduction activity of a prostaglandin E receptor in a prostaglandin E receptor-expressing cell that is contacted with the specified compound as compared to prostaglandin E receptor signal transduction activity in a prostaglandin E receptor-expressing cell that is not contacted with the specified compound. Assays that can be used to measure prostaglandin E receptor signal transduction are described, e.g., in WO 2010/108028, the disclosure of which is incorporated herein by reference as it pertains to methods of assessing prostaglandin E receptor signaling.

As used herein, the terms “administering,” “administration,” and the like refer to directly giving a patient a therapeutic agent (e.g., a population of cells, such as a population of pluripotent cells (e.g., embryonic stem cells, induced pluripotent stem cells, or CD34+ cells)) by any effective route. Exemplary routes of administration are described herein and include systemic administration routes, such as intravenous injection, among others.

As used herein, the term “allogeneic” refers to cells, tissues, nucleic acid molecules, or other substances obtained or derived from a different subject of the same species. For example, in the context of a population of cells (e.g., a population of pluripotent cells) expressing one or more proteins described herein, allogeneic cells include those that are (i) obtained from a subject that is not undergoing therapy and are then (ii) transduced or transfected with a vector that directs the expression of one or more desired proteins. The phrase “directs expression” refers to the inclusion of one or more polynucleotides encoding the one or more proteins to be expressed. The polynucleotide may contain additional sequence motifs that enhances expression of the protein of interest.

As used herein, the term “autologous” refers to cells, tissues, nucleic acid molecules, or other substances obtained or derived from an individual's own cells, tissues, nucleic acid molecules, or the like. For example, in the context of a population of cells (e.g., a population of pluripotent cells) expressing one or more proteins described herein, autologous cells include those that are obtained from the patient undergoing therapy that are then transduced or transfected with a vector that directs the expression of one or more proteins of interest.

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, “codon optimization” refers a process of modifying a nucleic acid sequence in accordance with the principle that the frequency of occurrence of synonymous codons (e.g., codons that code for the same amino acid) in coding DNA is biased in different species. Such codon degeneracy allows an identical polypeptide to be encoded by a variety of nucleotide sequences. Sequences modified in this way are referred to herein as “codon-optimized.” This process may be performed on any of the sequences described in this specification to enhance expression or stability. Codon optimization may be performed in a manner such as that described in, e.g., U.S. Pat. Nos. 7,561,972, 7,561,973, and 7,888,112, each of which is incorporated herein by reference in its entirety. The sequence surrounding the translational start site can be converted to a consensus Kozak sequence according to known methods. See, e.g., Kozak et al, Nucleic Acids Res. 15 (20): 8125-8148, incorporated herein by reference in its entirety. Multiple stop codons can be incorporated.

As used herein, the terms “condition” and “conditioning” refer to processes by which a subject is prepared for receipt of a transplant containing a population of cells (e.g., a population of pluripotent cells, such as CD34+ cells). Such procedures promote the engraftment of a cell transplant, for example, by selectively depleting endogenous cells (e.g., endogenous CD34+ cells, among others) thereby creating a vacancy which is in turn filled by the exogenous cell transplant. According to the methods described herein, a subject may be conditioned for cell transplant procedure by administration to the subject of one or more agents capable of ablating endogenous cells (e.g., CD34+ cells, among others), radiation therapy, or a combination thereof. Conditioning regimens useful in conjunction with the compositions and methods of the disclosure may be myeloablative or non-myeloablative. Other cell-ablating agents and methods well known in the art (e.g., antibodies and antibody-drug conjugates) may also be used.

As used herein, the terms “conservative mutation,” “conservative substitution,” “conservative amino acid substitution,” and the like refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties of naturally occurring amino acids

| | | | | Electrostatic | |
|---|---|---|---|---|---|
| | | | Side- | character at | |
| | 3 Letter | 1 Letter | chain | physiological pH | Steric |
| Amino Acid | Code | Code | Polarity | (7.4) | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms in | |
| | | | | equilibrium at pH 7.4 | |
| Isoleucine | Ile | I | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein in the context of a gene of interest, the term “disrupt” refers to preventing the formation of a functional gene product. A gene product is considered to be functional according to the present disclosure if it fulfills its normal (wild type) function(s). Disruption of the gene prevents expression of a functional factor (e.g., protein) encoded by the gene and may be achieved, for example, by way of an insertion, deletion, or substitution of one or more bases in a sequence encoded by the gene and/or a promoter and/or an operator that is necessary for expression of the gene in a subject. The disrupted gene may be disrupted by, e.g., removal of at least a portion of the gene from a genome of the subject, alteration of the gene to prevent expression of a functional factor (e.g., protein) encoded by the gene, an interfering RNA, or expression of a dominant negative factor by an exogenous gene. Materials and methods for genetically modifying cells (e.g., pluripotent cells, such as CD34+ cells, hematopoietic stem cells, and myeloid progenitor cells, among others) so as to disrupt the expression of one or more genes are detailed, for example, in U.S. Pat. Nos. 8,518,701; 9,499,808; and US 2012/0222143, the disclosures of each of which are incorporated herein by reference in their entirety (in case of conflict, the instant specification is controlling).

As used herein, the terms “embryonic stem cell” and “ES cell” refer to an embryo-derived totipotent or pluripotent stem cell, derived from the inner cell mass of a blastocyst that can be maintained in an in vitro culture under suitable conditions. ES cells are capable of differentiating into cells of any of the three vertebrate germ layers, e.g., the endoderm, the ectoderm, or the mesoderm. ES cells are also characterized by their ability to propagate indefinitely under suitable in vitro culture conditions. ES cells are described, for example, in Thomson et al., Science 282:1145 (1998), the disclosure of which is incorporated herein by reference as it pertains to the structure and functionality of embryonic stem cells.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein, the term “expansion agent” refers to a substance capable of promoting the proliferation of a given cell type ex vivo. Accordingly, a “hematopoietic stem cell expansion agent” or an “HSC expansion agent” refers to a substance capable of promoting the proliferation of a population of hematopoietic stem cells ex vivo. Hematopoietic stem cell expansion agents include those that effectuate the proliferation of a population of hematopoietic stem cells such that the cells retain hematopoietic stem cell functional potential. Exemplary hematopoietic stem cell expansion agents that may be used in conjunction with the compositions and methods of the disclosure include, without limitation, aryl hydrocarbon receptor antagonists, such as those described in U.S. Pat. Nos. 8,927,281 and 9,580,426, the disclosures of each of which are incorporated herein by reference in their entirety, and, in particular, compound SR1. Additional hematopoietic stem cell expansion agents that may be used in conjunction with the compositions and methods of the disclosure include compound UM-171 and other compounds described in U.S. Pat. No. 9,409,906, the disclosure of which is incorporated herein by reference in its entirety. Hematopoietic stem cell expansion agents further include structural and/or stereoisomeric variants of compound UM-171, such as the compounds described in US 2017/0037047, the disclosure of which is incorporated herein by reference in its entirety. Additional hematopoietic stem cell expansion agents suitable for use in the instant disclosure include histone deacetylase (HDAC) inhibitors, such as trichostatin A, trapoxin, trapoxin A, chlamydocin, sodium butyrate, dimethyl sulfoxide, suberanilohydroxamic acid, m-carboxycinnamic acid bishydroxamide, HC-toxin, Cyl-2, WF-3161, depudecin, and radicicol, among others described, for example, in WO 2000/023567, the disclosure of which is incorporated herein by reference.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. In the context of a gene that encodes a protein product, the terms “gene expression” and the like are used interchangeably with the terms “protein expression” and the like. Expression of a gene or protein of interest in a subject can manifest, for example, by detecting: an increase in the quantity or concentration of mRNA encoding corresponding protein (as assessed, e.g., using RNA detection procedures described herein or known in the art, such as quantitative polymerase chain reaction (qPCR) and RNA seq techniques), an increase in the quantity or concentration of the corresponding protein (as assessed, e.g., using protein detection methods described herein or known in the art, such as enzyme-linked immunosorbent assays (ELISA), among others), and/or an increase in the activity of the corresponding protein (e.g., in the case of an enzyme, as assessed using an enzymatic activity assay described herein or known in the art) in a sample obtained from the subject. As used herein, a cell is considered to “express” a gene or protein of interest if one or more, or all, of the above events can be detected in the cell or in a medium in which the cell resides. For example, a gene or protein of interest is considered to be “expressed” by a cell or population of cells if one can detect (i) production of a corresponding RNA transcript, such as an mRNA template, by the cell or population of cells (e.g., using RNA detection procedures described herein); (ii) processing of the RNA transcript (e.g., splicing, editing, 5′ cap formation, and/or 3′ end processing, such as using RNA detection procedures described herein); (iii) translation of the RNA template into a protein product (e.g., using protein detection procedures described herein); and/or (iv) post-translational modification of the protein product (e.g., using protein detection procedures described herein).

As used herein, the term “functional potential” as it pertains to a pluripotent cell, such as a hematopoietic stem cell, refers to the functional properties of stem cells which include: 1) multi-potency (which refers to the ability to differentiate into multiple different blood lineages including, but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells); 2) self-renewal (which refers to the ability of stem cells to give rise to daughter cells that have equivalent potential as the mother cell, and further that this ability can repeatedly occur throughout the lifetime of an individual without exhaustion); and 3) the ability of stem cells or progeny thereof to be reintroduced into a transplant recipient whereupon they home to the stem cell niche and re-establish productive and sustained cell growth and differentiation.

As used herein, the terms “hematopoietic stem cells” and “HSCs” refer to immature blood cells having the capacity to self-renew and to differentiate into mature blood cells of diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). It is known in the art that such cells may or may not include CD34+ cells. CD34+ cells are immature cells that express the CD34 cell surface marker. In humans, CD34+ cells are believed to include a subpopulation of cells with the stem cell properties defined above, whereas in mice, HSCs are CD34−. In addition, HSCs also refer to long term repopulating HSC (LT-HSC) and short-term repopulating HSC (ST-HSC). LT-HSC and ST-HSC are differentiated, based on functional potential and on cell surface marker expression. For example, human HSC can be CD34+, CD38−, CD45RA−, CD90+, CD49F+, and lin− (negative for mature lineage markers including CO2, CD3, CD4, CD7, CD8, CD10, CD11B, CD19, CD20, CD56, CD235A). In mice, bone marrow LT-HSC can be CD34−, SCA-1+, C-kit+, CD135−, Slamf1/CD150+, CD48−, and lin− (negative for mature lineage markers including Ter119, CD11b, Gr1, CD3, CD4, CD8, B220, IL-7ra), whereas ST-HSC can be CD34+, SCA-1+, C-kit+, CD135−, Slamf1/CD150+, and lin− (negative for mature lineage markers including Ter119, CD11b, Gr1, CD3, CD4, CD8, B220, IL-7ra). In addition, ST-HSC are less quiescent (i.e., more active) and more proliferative than L T-HSC under homeostatic conditions. However, LT-HSC have greater self-renewal potential (i.e., they survive throughout adulthood, and can be serially transplanted through successive recipients), whereas ST-HSC have limited self-renewal (i.e., they survive for only a limited period of time, and do not possess serial transplantation potential). Any of these HSCs can be used in any of the methods described herein. Optionally, ST-HSCs are useful because they are highly proliferative and thus, can more quickly give rise to differentiated progeny.

As used herein, an agent that inhibits histone deacetylation refers to a substance or composition (e.g., a small molecule, protein, interfering RNA, messenger RNA, or other natural or synthetic compound, or a composition such as a virus or other material composed of multiple substances) capable of attenuating or preventing the activity of histone deacetylase, more particularly its enzymatic activity either via direct interaction or via indirect means such as by causing a reduction in the quantity of a histone deacetylase produced in a cell or by inhibition of the interaction between a histone deacetylase and an acetylated histone substrate. Inhibiting histone deacetylase enzymatic activity means reducing the ability of a histone deacetylase to catalyze the removal of an acetyl group from a histone residue (e.g., a mono-, di-, or tri-methylated lysine residue; a monomethylated arginine residue, or a symmetric/asymmetric dimethylated arginine residue, within a histone protein). Preferably, such inhibition is specific, such that the agent that inhibits histone deacetylation reduces the ability of a histone deacetylase to remove an acetyl group from a histone residue at a concentration that is lower than the concentration of the inhibitor that is required to produce another, unrelated biological effect.

As used herein, the terms “histone deacetylase” and “HDAC” refer to any one of a family of enzymes that catalyze the removal of acetyl groups from the ε-amino groups of lysine residues at the N-terminus of a histone. Unless otherwise indicated by context, the term “histone” is meant to refer to any histone protein, including HI, H2A, H2B, H3, H4, and H5, from any species. Human HDAC proteins or gene products, include, but are not limited to, HDAC-1, HDAC-2, HDAC-3, HDAC-4, HDAC-5, HDAC-6, HDAC-7, HDAC-8, HDAC-9, HDAC-10, and HDAC-11.

As used herein, the term “HLA-matched” refers to a donor-recipient pair in which none of the HLA antigens are mismatched between the donor and recipient, such as a donor providing a hematopoietic stem cell graft to a recipient in need of hematopoietic stem cell transplant therapy. HLA-matched (i.e., where all of the 6 alleles are matched) donor-recipient pairs have a decreased risk of graft rejection, as endogenous T cells and NK cells are less likely to recognize the incoming graft as foreign, and, are thus less likely to mount an immune response against the transplant.

As used herein, the term “HLA-mismatched” refers to a donor-recipient pair in which at least one HLA antigen, in particular with respect to HLA-A, HLA-B, HLA-C, and HLA-DR, is mismatched between the donor and recipient, such as a donor providing a hematopoietic stem cell graft to a recipient in need of hematopoietic stem cell transplant therapy. In some embodiments, one haplotype is matched and the other is mismatched. HLA-mismatched donor-recipient pairs may have an increased risk of graft rejection relative to HLA-matched donor-recipient pairs, as endogenous T cells and NK cells are more likely to recognize the incoming graft as foreign in the case of an HLA-mismatched donor-recipient pair, and such T cells and NK cells are thus more likely to mount an immune response against the transplant.

As used herein, the terms “induced pluripotent stem cell,” “iPS cell,” and “iPSC” refer to a pluripotent stem cell that can be derived directly from a differentiated somatic cell. Human iPS cells can be generated by introducing specific sets of reprogramming factors into a non-pluripotent cell that can include, for example, Oct3/4, Sox family transcription factors (e.g., Sox1, Sox2, Sox3, Soxl5), Myc family transcription factors (e.g., c-Myc, 1-Myc, n-Myc), Kruppel-like family (KLF) transcription factors (e.g., KLF1, KLF2, KLF4, KLF5), and/or related transcription factors, such as NANOG, LIN28, and/or Glis1. Human iPS cells can also be generated, for example, by the use of miRNAs, small molecules that mimic the actions of transcription factors, or lineage specifiers. Human iPS cells are characterized by their ability to differentiate into any cell of the three vertebrate germ layers, e.g., the endoderm, the ectoderm, or the mesoderm. Human iPS cells are also characterized by their ability propagate indefinitely under suitable in vitro culture conditions. Human iPS cells are described, for example, in Takahashi and Yamanaka, Cell 126:663 (2006), the disclosure of which is incorporated herein by reference as it pertains to the structure and functionality of iPS cells.

As used herein, the term “inhibitor” refers to an agent (e.g., a small molecule, peptide fragment, protein, antibody, or antigen-binding fragment thereof) that binds to, and/or otherwise suppresses the activity of, a target molecule.

As used herein in the context of hematopoietic stem and/or progenitor cells, the term “mobilization” refers to release of such cells from a stem cell niche where the cells typically reside (e.g., the bone marrow) into peripheral circulation. “Mobilization agents” are agents that are capable of inducing the release of hematopoietic stem and/or progenitor cells from a stem cell niche into peripheral circulation.

As used herein, the term “myeloablative” or “myeloablation” refers to a conditioning regiment that substantially impairs or destroys the hematopoietic system, typically by exposure to a cytotoxic agent or radiation. Myeloablation encompasses complete myeloablation brought on by high doses of cytotoxic agent or total body irradiation that destroys the hematopoietic system.

As used herein, the term “non-myeloablative” or “myelosuppressive” refers to a conditioning regiment that does not eliminate substantially all hematopoietic cells of host origin.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “pharmaceutically acceptable” refers to those compounds, materials, compositions and/or dosage forms, which are suitable for contact with the tissues of a subject, such as a mammal (e.g., a human) without excessive toxicity, irritation, allergic response and other problem complications commensurate with a reasonable benefit/risk ratio.

As used herein, the term “pharmaceutical composition” refers to a composition containing a therapeutic agent (e.g., an agent that increases NOD2 activity and/or expression to physiologically normal levels) that may be administered to a subject, such as a mammal, e.g., a human, in order to prevent, treat or control a particular disease or condition affecting the mammal, such as Crohn's disease as described herein.

As used herein, the term “pluripotent cell” refers to a cell that possesses the ability to develop into more than one differentiated cell type, such as a cell type of the hematopoietic lineage (e.g., granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Examples of pluripotent cells are ESCs, iPSCs, and CD34+ cells.

As used herein, the term “promoter” refers to a recognition site on DNA that is bound by an RNA polymerase. The polymerase drives transcription of the transgene. Exemplary promoters suitable for use with the compositions and methods described herein are described, for example, in Sandelin et al., Nature Reviews Genetics 8:424 (2007), the disclosure of which is incorporated herein by reference as it pertains to nucleic acid regulatory elements. Additionally, the term “promoter” may refer to a synthetic promoter, which are regulatory DNA sequences that do not occur naturally in biological systems. Synthetic promoters contain parts of naturally occurring promoters combined with polynucleotide sequences that do not occur in nature and can be optimized to express recombinant DNA using a variety of transgenes, vectors, and target cell types.

As used herein, the term “tissue-specific promoter” refers to a promoter that selectively facilitates the expression of a gene of interest in a particular cell type or tissue type. Examples of tissue-specific promoters that may be used in conjunction with the compositions and methods of the disclosure include a sp146/p47 promoter, CD11b promoter, CD68 promoter, and a sp146/gp9 promoter, among others.

As used herein, the term “ubiquitous promoter” refers to a promoter that facilitates the expression of a gene of interest in a variety of cell types or tissue types, such as a promoter that does not exhibit a preference for facilitating gene expression in one cell type over another or in one tissue type over another. Examples of ubiquitous promoters that may be used in conjunction with the compositions and methods of the disclosure include an elongation factor 1-alpha promoter, among others.

As used herein, the term “plasmid” refers to a to an extrachromosomal circular double stranded DNA molecule into which additional DNA segments may be ligated. A plasmid is a type of vector, a nucleic acid molecule capable of transporting another nucleic acid to which it has been linked. Certain plasmids are capable of autonomous replication in a host cell into which they are introduced (e.g., bacterial plasmids having a bacterial origin of replication and episomal mammalian plasmids). Other vectors (e.g., non-episomal mammalian vectors) can be integrated into the genome of a host cell upon introduction into the host cell, and thereby are replicated along with the host genome. Certain plasmids are capable of directing the expression of genes to which they are operably linked.

As used herein, a therapeutic agent is considered to be “provided” to a patient if the patient is directly administered the therapeutic agent or if the patient is administered a substance that is processed or metabolized in vivo so as to yield the therapeutic agent endogenously. For example, a patient, such as a patient having Crohn's disease as described herein, may be provided a protein of the disclosure (e.g., functional NOD2) by direct administration of the protein or by administration of a substance (e.g., a NOD2 gene) that is processed or metabolized in vivo so as to yield the desired protein endogenously. Additional examples of “providing” a protein of interest to a patient are instances in which the patient is administered (i) a nucleic acid molecule encoding the protein of interest, (ii) a vector (e.g., a viral vector) containing such a nucleic acid molecule, (iii) a cell or population of cells containing such a vector or nucleic acid molecule, (iv) an interfering RNA molecule, such as a siRNA, shRNA, or miRNA molecule, that stimulates expression of the protein endogenously upon administration to the patient, or (v) a protein precursor that is processed, for example, by way of one or more post-translational modifications, to yield the desired protein endogenously.

As used herein, the term “regulatory sequence” includes promoters, enhancers and other expression control elements (e.g., polyadenylation signals) that control the transcription or translation of the gene(s). Such regulatory sequences are described, for example, in Perdew et al., Regulation of Gene Expression (Humana Press, New York, N.Y., (2014)); incorporated herein by reference.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) isolated from a subject. The term sample can also relate to a prepared or processed samples, such as a mRNA- or cDNA-containing sample.

As used herein, the term “splice variant” refers to a transcribed product (i.e. RNA) of a single gene that can be processed to produce different mRNA molecules as a result of alternative inclusion or exclusion of specific exons (e.g. exon skipping) within the precursor mRNA. Proteins produced from translation of specific splice variants may differ in their structure and biological activity.

As used herein, the terms “stem cell” and “undifferentiated cell” refer to a cell in an undifferentiated or partially differentiated state that has the developmental potential to differentiate into multiple cell types. A stem cell is capable of proliferation and giving rise to more such stem cells while maintaining its functional potential. Stem cells can divide asymmetrically, which is known as obligatory asymmetrical differentiation, with one daughter cell retaining the functional potential of the parent stem cell and the other daughter cell expressing some distinct other specific function, phenotype and/or developmental potential from the parent cell. The daughter cells themselves can be induced to proliferate and produce progeny that subsequently differentiate into one or more mature cell types, while also retaining one or more cells with parental developmental potential. A differentiated cell may derive from a multipotent cell, which itself is derived from a multipotent cell, and so on. Alternatively, some of the stem cells in a population can divide symmetrically into two stem cells. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating. In some embodiments, the term stem cell refers generally to a naturally occurring parent cell whose descendants (progeny cells) specialize, often in different directions, by differentiation, e.g., by acquiring completely individual characters, as occurs in progressive diversification of embryonic cells and tissues. Some differentiated cells also have the capacity to give rise to cells of greater developmental potential. Such capacity may be natural or may be induced artificially upon treatment with various factors. Cells that begin as stem cells might proceed toward a differentiated phenotype, but then can be induced to “reverse” and re-express the stem cell phenotype, a term often referred to as “dedifferentiation” or “reprogramming” or “retrodifferentiation” by persons of ordinary skill in the art.

As used herein, the term “transgene” refers to a recombinant nucleic acid (e.g., DNA or cDNA) encoding a gene product (e.g., a gene product described herein). The gene product may be an RNA, peptide, or protein. In addition to the coding region for the gene product, the transgene may include or be operably linked to one or more elements to facilitate or enhance expression, such as a promoter, enhancer(s), destabilizing domain(s), response element(s), reporter element(s), insulator element(s), polyadenylation signal(s), and/or other functional elements. Embodiments of the disclosure may utilize any known suitable promoter, enhancer(s), destabilizing domain(s), response element(s), reporter element(s), insulator element(s), polyadenylation signal(s), and/or other functional elements.

As used herein, the term “transfection” refers to any of a wide variety of techniques commonly used for the introduction of exogenous DNA into a prokaryotic or eukaryotic host cell, e.g., electroporation, lipofection, calcium-phosphate precipitation, DEAE-dextran transfection, Nucleofection, squeeze-poration, sonoporation, optical transfection, Magnetofection, impalefection, and the like.

As used herein, the terms “subject” and “patient” are used interchangeably and refer to an organism (e.g., a mammal, such as a human) that is at risk of developing or has been diagnosed as having, and/or is undergoing treatment for, a disease, such as Crohn's disease as described herein.

As used herein, the terms “transduction” and “transduce” refer to a method of introducing a viral vector construct or a part thereof into a cell and subsequent expression of a transgene encoded by the vector construct or part thereof in the cell.

As used herein, “treatment” and “treating” refer to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those prone to or at risk of developing the condition or disorder, as well as those in which the condition or disorder is to be prevented.

As used herein, the term “vector” includes a nucleic acid vector, e.g., a DNA vector, such as a plasmid, an RNA vector, virus, or other suitable replicon (e.g., viral vector). A variety of vectors have been developed for the delivery of polynucleotides encoding exogenous proteins into a prokaryotic or eukaryotic cell. Examples of such expression vectors are disclosed in, e.g., WO 1994/011026; incorporated herein by reference as it pertains to vectors suitable for the expression of a gene of interest. Expression vectors suitable for use with the compositions and methods described herein contain a polynucleotide sequence as well as, e.g., additional sequence elements used for the expression of proteins and/or the integration of these polynucleotide sequences into the genome of a mammalian cell. Vectors that can be used for the expression of a protein or proteins described herein include plasmids that contain regulatory sequences, such as promoter and enhancer regions, which direct gene transcription. Additionally, useful vectors for expression of a protein or proteins described herein may contain polynucleotide sequences that enhance the rate of translation of the corresponding gene or genes or improve the stability or nuclear export of the mRNA that results from gene transcription. Examples of such sequence elements are 5′ and 3′ untranslated regions, an IRES, and a polyadenylation signal site in order to direct efficient transcription of a gene or genes carried on an expression vector. Expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, or zeocin, among others.

As used herein in the context of providing a therapeutic agent to a patient (e.g., a patient having Crohn's disease), the terms “Nucleotide-binding oligomerization domain-containing protein 2” and its abbreviation, “NOD2,” are used interchangeably and refer to the gene encoding NOD2, or the corresponding protein product, as context will dictate. The terms “Nucleotide-binding oligomerization domain-containing protein 2” and “NOD2” embrace wild-type forms of the NOD2 gene or protein, as well as variants (e.g., splice variants, truncations, concatemers, and fusion constructs, among others) of wild-type NOD2 proteins and nucleic acids encoding the same.

As used herein, the term “functional NOD2” refers to a wild-type form of the NOD2 gene or protein, as well as variants (e.g., splice variants, truncations, concatemers, and fusion constructs, among others) of wild-type NOD2 proteins and nucleic acids encoding the same, so long as such variants retain normal, physiological abilities of wild-type NOD2, such as the ability to detect bacterial peptidoglycans, particularly muramyl dipeptide (MDP), and/or to stimulate an immune response thereto. Examples of such variants may include proteins having at least 70% sequence identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 99.9% identity, or more) to any of the amino acid sequences of a wild-type NOD2 protein (e.g., SEQ ID NO: 2), such as variants having an amino acid sequence that differs from that of wild-type NOD2 by way of one or more conservative amino acid substitutions, provided that the NOD2 variant retains the therapeutic function of a wild-type NOD2.

SEQ ID NO: 2 corresponds to UniProt reference sequence Q9HC29-1, and is shown below:

(SEQ ID NO: 2)
MGEEGGSASHDEEERASVLLGHSPGCEMCSQEAFQ
AQRSQLVELLVSGSLEGFESVLDWLLSWEVLSWED
YEGFHLLGQPLSHLARRLLDTVWNKGTWACQKLIA
AAQEAQADSQSPKLHGCWDPHSLHPARDLQSHRPA
IVRRLHSHVENMLDLAWERGFVSQYECDEIRLPIF
TPSQRARRLLDLATVKANGLAAFLLQHVQELPVPL
ALPLEAATCKKYMAKLRTTVSAQSRFLSTYDGAET
LCLEDIYTENVLEVWADVGMAGPPQKSPATLGLEE
LFSTPGHLNDDADTVLVVGEAGSGKSTLLQRLHLL
WAAGQDFQEFLFVFPFSCRQLQCMAKPLSVRTLLF
EHCCWPDVGQEDIFQLLLDHPDRVLLTFDGFDEFK
FRFTDRERHCSPTDPTSVQTLLFNLLQGNLLKNAR
KVVTSRPAAVSAFLRKYIRTEFNLKGFSEQGIELY
LRKRHHEPGVADRLIRLLQETSALHGLCHLPVFSW
MVSKCHQELLLQEGGSPKTTTDMYLLILQHFLLHA
TPPDSASQGLGPSLLRGRLPTLLHLGRLALWGLGM
CCYVFSAQQLQAAQVSPDDISLGFLVRAKGVVPGS
TAPLEFLHITFQCFFAAFYLALSADVPPALLRHLF
NCGRPGNSPMARLLPTMCIQASEGKDSSVAALLQK
AEPHNLQITAAFLAGLLSREHWGLLAECQTSEKAL
LRRQACARWCLARSLRKHFHSIPPAAPGEAKSVHA
MPGFIWLIRSLYEMQEERLARKAARGLNVGHLKLT
FCSVGPTECAALAFVLQHLRRPVALQLDYNSVGDI
GVEQLLPCLGVCKALYLRDNNISDRGICKLIECAL
HCEQLQKLALFNNKLTDGCAHSMAKLLACRQNFLA
LRLGNNYITAAGAQVLAEGLRGNTSLQFLGFWGNR
VGDEGAQALAEALGDHQSLRWLSLVGNNIGSVGAQ
ALALMLAKNVMLEELCLEENHLQDEGVCSLAEGLK
KNSSLKILKLSNNCITYLGAEALLOALERNDTILE
VWLRGNTFSLEEVDKLGCRDTRLLL

An exemplary NOD2 nucleic acid sequence is European Nucleotide Archive reference sequence AF178930.1, which corresponds to SEQ ID NO: 1, shown below:

(SEQ ID NO: 1)
GTAGACAGATCCAGGCTCACCAGTCCTGTGCCACT
GGGCTTTTGGCGTTCTGCACAAGGCCTACCCGCAG
ATGCCATGCCTGCTCCCCCAGCCTAATGGGCTTTG
ATGGGGGAAGAGGGTGGTTCAGCCTCTCACGATGA
GGAGGAAAGAGCAAGTGTCCTCCTCGGACATTCTC
CGGGTTGTGAAATGTGCTCGCAGGAGGCTTTTCAG
GCACAGAGGAGCCAGCTGGTCGAGCTGCTGGTCTC
AGGGTCCCTGGAAGGCTTCGAGAGTGTCCTGGACT
GGCTGCTGTCCTGGGAGGTCCTCTCCTGGGAGGAC
TACGAGGGCTTCCACCTCCTGGGCCAGCCTCTCTC
CCACTTGGCCAGGCGCCTTCTGGACACCGTCTGGA
ATAAGGGTACTTGGGCCTGTCAGAAGCTCATCGCG
GCTGCCCAAGAAGCCCAGGCCGACAGCCAGTCCCC
CAAGCTGCATGGCTGCTGGGACCCCCACTCGCTCC
ACCCAGCCCGAGACCTGCAGAGTCACCGGCCAGCC
ATTGTCAGGAGGCTCCACAGCCATGTGGAGAACAT
GCTGGACCTGGCATGGGAGCGGGGTTTCGTCAGCC
AGTATGAATGTGATGAAATCAGGTTGCCGATCTTC
ACACCGTCCCAGAGGGCAAGAAGGCTGCTTGATCT
TGCCACGGTGAAAGCGAATGGATTGGCTGCCTTCC
TTCTACAACATGTTCAGGAATTACCAGTCCCATTG
GCCCTGCCTTTGGAAGCTGCCACATGCAAGAAGTA
TATGGCCAAGCTGAGGACCACGGTGTCTGCTCAGT
CTCGCTTCCTCAGTACCTATGATGGAGCAGAGACG
CTCTGCCTGGAGGACATATACACAGAGAATGTCCT
GGAGGTCTGGGCAGATGTGGGCATGGCTGGACCCC
CGCAGAAGAGCCCAGCCACCCTGGGCCTGGAGGAG
CTCTTCAGCACCCCTGGCCACCTCAATGACGATGC
GGACACTGTGCTGGTGGTGGGTGAGGCGGGCAGTG
GCAAGAGCACGCTCCTGCAGCGGCTGCACTTGCTG
TGGGCTGCAGGGCAAGACTTCCAGGAATTTCTCTT
TGTCTTCCCATTCAGCTGCCGGCAGCTGCAGTGCA
TGGCCAAACCACTCTCTGTGCGGACTCTACTCTTT
GAGCACTGCTGTTGGCCTGATGTTGGTCAAGAAGA
CATCTTCCAGTTACTCCTTGACCACCCTGACCGTG
TCCTGTTAACCTTTGATGGCTTTGACGAGTTCAAG
TTCAGGTTCACGGATCGTGAACGCCACTGCTCCCC
GACCGACCCCACCTCTGTCCAGACCCTGCTCTTCA
ACCTTCTGCAGGGCAACCTGCTGAAGAATGCCCGC
AAGGTGGTGACCAGCCGTCCGGCCGCTGTGTCGGC
GTTCCTCAGGAAGTACATCCGCACCGAGTTCAACC
TCAAGGGCTTCTCTGAACAGGGCATCGAGCTGTAC
CTGAGGAAGCGCCATCATGAGCCCGGGGTGGCGGA
CCGCCTCATCCGCCTGCTCCAAGAGACCTCAGCCC
TGCACGGTTTGTGCCACCTGCCTGTCTTCTCATGG
ATGGTGTCCAAATGCCACCAGGAACTGTTGCTGCA
GGAGGGGGGGTCCCCAAAGACCACTACAGATATGT
ACCTGCTGATTCTGCAGCATTTTCTGCTGCATGCC
ACCCCCCCAGACTCAGCTTCCCAAGGTCTGGGACC
CAGTCTTCTTCGGGGCCGCCTCCCCACCCTCCTGC
ACCTGGGCAGACTGGCTCTGTGGGGCCTGGGCATG
TGCTGCTACGTGTTCTCAGCCCAGCAGCTCCAGGC
AGCACAGGTCAGCCCTGATGACATTTCTCTTGGCT
TCCTGGTGCGTGCCAAAGGTGTCGTGCCAGGGAGT
ACGGCGCCCCTGGAATTCCTTCACATCACTTTCCA
GTGCTTCTTTGCCGCGTTCTACCTGGCACTCAGTG
CTGATGTGCCACCAGCTTTGCTCAGACACCTCTTC
AATTGTGGCAGGCCAGGCAACTCACCAATGGCCAG
GCTCCTGCCCACGATGTGCATCCAGGCCTCGGAGG
GAAAGGACAGCAGCGTGGCAGCTTTGCTGCAGAAG
GCCGAGCCGCACAACCTTCAGATCACAGCAGCCTT
CCTGGCAGGGCTGTTGTCCCGGGAGCACTGGGGCC
TGCTGGCTGAGTGCCAGACATCTGAGAAGGCCCTG
CTCCGGCGCCAGGCCTGTGCCCGCTGGTGTCTGGC
CCGCAGCCTCCGCAAGCACTTCCACTCCATCCCGC
CAGCTGCACCGGGTGAGGCCAAGAGCGTGCATGCC
ATGCCCGGGTTCATCTGGCTCATCCGGAGCCTGTA
CGAGATGCAGGAGGAGCGGCTGGCTCGGAAGGCTG
CACGTGGCCTGAATGTTGGGCACCTCAAGTTGACA
TTTTGCAGTGTGGGCCCCACTGAGTGTGCTGCCCT
GGCCTTTGTGCTGCAGCACCTCCGGCGGCCCGTGG
CCCTGCAGCTGGACTACAACTCTGTGGGTGACATT
GGCGTGGAGCAGCTGCTGCCTTGCCTTGGTGTCTG
CAAGGCTCTGTATTTGCGCGATAACAATATCTCAG
ACCGAGGCATCTGCAAGCTCATTGAATGTGCTCTT
CACTGCGAGCAATTGCAGAAGTTAGCTCTATTCAA
CAACAAATTGACTGACGGCTGTGCACACTCCATGG
CTAAGCTCCTTGCATGCAGGCAGAACTTCTTGGCA
TTGAGGCTGGGGAATAACTACATCACTGCCGCGGG
AGCCCAAGTGCTGGCCGAGGGGCTCCGAGGCAACA
CCTCCTTGCAGTTCCTGGGATTCTGGGGCAACAGA
GTGGGTGACGAGGGGGCCCAGGCCCTGGCTGAAGC
CTTGGGTGATCACCAGAGCTTGAGGTGGCTCAGCC
TGGTGGGGAACAACATTGGCAGTGTGGGTGCCCAA
GCCTTGGCACTGATGCTGGCAAAGAACGTCATGCT
AGAAGAACTCTGCCTGGAGGAGAACCATCTCCAGG
ATGAAGGTGTATGTTCTCTCGCAGAAGGACTGAAG
AAAAATTCAAGTTTGAAAATCCTGAAGTTGTCCAA
TAACTGCATCACCTACCTAGGGGCAGAAGCCCTCC
TGCAGGCCCTTGAAAGGAATGACACCATCCTGGAA
GTCTGGCTCCGAGGGAACACTTTCTCTCTAGAGGA
GGTTGACAAGCTCGGCTGCAGGGACACCAGACTCT
TGCTTTGAAGTCTCCGGGAGGATGTTCGTCTCAGT
TTGTTTGTGAGCAGGCTGTGAGTTTGGGCCCCAGA
GGCTGGGTGACATGTGTTGGCAGCCTCTTCAAAAT
GAGCCCTGTCCTGCCTAAGGCTGAACTTGTTTTCT
GGGAACACCATAGGTCACCTTTATTCTGGCAGAGG
AGGGAGCATCAGTGCCCTCCAGGATAGACTTTTCC
CAAGCCTACTTTTGCCATTGACTTCTTCCCAAGAT
TCAATCCCAGGATGTACAAGGACAGCCCCTCCTCC
ATAGTATGGGACTGGCCTCTGCTGATCCTCCCAGG
CTTCCGTGTGGGTCAGTGGGGCCCATGGATGTGCT
TGTTAACTGAGTGCCTTTTGGTGGAGAGGCCCGGC
CTCTCACAAAAGACCCCTTACCACTGCTCTGATGA
AGAGGAGTACACAGAACACATAATTCAGGAAGCAG
CTTTCCCCATGTCTCGACTCATCCATCCAGGCCAT
TCCCCGTCTCTGGTTCCTCCCCTCCTCCTGGACTC
CTGCACACGCTCCTTCCTCTGAGGCTGAAATTCAG
AATATTAGTGACCTCAGCTTTGATATTTCACTTAC
AGCACCCCCAACCCTGGCACCCAGGGTGGGAAGGG
CTACACCTTAGCCTGCCCTCCTTTCCGGTGTTTAA
GACATTTTTGGAAGGGGACACGTGACAGCCGTTTG
TTCCCCAAGACATTCTAGGTTTGCAAGAAAAATAT
GACCACACTCCAGCTGGGATCACATGTGGACTTTT
ATTTCCAGTGAAATCAGTTACTCTTCAGTTAAGCC
TTTGGAAACAGCTCGACTTTAAAAAGCTCCAAATG
CAGCTTTAAAAAATTAATCTGGGCCAGAATTTCAA
ACGGCCTCACTAGGCTTCTGGTTGATGCCTGTGAA
CTGAACTCTGACAACAGACTTCTGAAATAGACCCA
CAAGAGGCAGTTCCATTTCATTTGTGCCAGAATGC
TTTAGGATGTACAGTTATGGATTGAAAGTTTACAG
GAAAAAAAATTAGGCCGTTCCTTCAAAGCAAATGT
CTTCCTGGATTATTCAAAATGATGTATGTTGAAGC
CTTTGTAAATTGTCAGATGCTGTGCAAATGTTATT
ATTTTAAACATTATGATGTGTGAAAACTGGTTAAT
ATTTATAGGTCACTTTGTTTTACTGTCTTAAGTTT
ATACTCTTATAGACAACATGGCCGTGAACTTTATG
CTGTAAATAATCAGAGGGGAATAAACTGTTGAGTC
AAAAC

As used herein, an agent that “increases expression and/or activity of NOD2” refers to an agent that, upon administration to a patient (e.g., a human patient having Crohn's disease as described herein) facilitates expression of functional NOD2 at physiologically normal levels. Thus, increased expression or activity of NOD2 is relative to the amount present in the patient before treatment with the agent. For example, an agent that “increases expression and/or activity of NOD2” includes one that, upon administration to a human patient having Crohn's disease as described herein, effectuates expression of functional NOD2 at a level of from about 20% to about 200% of functional NOD2 expression observed in a human subject of comparable age and body mass index that does not have Crohn's disease. The agent may, for example, effectuate expression of functional NOD2 at a level of about 20% of that observed in a human subject of comparable age and body mass index that does not have Crohn's disease. In some embodiments, the agent effectuates expression of functional NOD2 at a level of about 30% of that observed in a human subject of comparable age and body mass index that does not have Crohn's disease. In some embodiments, the agent effectuates expression of functional NOD2 at a level of about 40% of that observed in a human subject of comparable age and body mass index that does not have Crohn's disease. In some embodiments, the agent effectuates expression of functional NOD2 at a level of about 50% of that observed in a human subject of comparable age and body mass index that does not have Crohn's disease. In some embodiments, the agent effectuates expression of functional NOD2 at a level of about 60% of that observed in a human subject of comparable age and body mass index that does not have Crohn's disease. In some embodiments, the agent effectuates expression of functional NOD2 at a level of about 70% of that observed in a human subject of comparable age and body mass index that does not have Crohn's disease. In some embodiments, the agent effectuates expression of functional NOD2 at a level of about 80% of that observed in a human subject of comparable age and body mass index that does not have Crohn's disease. In some embodiments, the agent effectuates expression of functional NOD2 at a level of about 90% of that observed in a human subject of comparable age and body mass index that does not have Crohn's disease. In some embodiments, the agent effectuates expression of functional NOD2 at a level of about 100% of that observed in a human subject of comparable age and body mass index that does not have Crohn's disease. In some embodiments, the agent effectuates expression of functional NOD2 at a level of about 110% of that observed in a human subject of comparable age and body mass index that does not have Crohn's disease. In some embodiments, the agent effectuates expression of functional NOD2 at a level of about 120% of that observed in a human subject of comparable age and body mass index that does not have Crohn's disease. In some embodiments, the agent effectuates expression of functional NOD2 at a level of about 130% of that observed in a human subject of comparable age and body mass index that does not have Crohn's disease. In some embodiments, the agent effectuates expression of functional NOD2 at a level of about 140% of that observed in a human subject of comparable age and body mass index that does not have Crohn's disease. In some embodiments, the agent effectuates expression of functional NOD2 at a level of about 150% of that observed in a human subject of comparable age and body mass index that does not have Crohn's disease. In some embodiments, the agent effectuates expression of functional NOD2 at a level of about 160% of that observed in a human subject of comparable age and body mass index that does not have Crohn's disease. In some embodiments, the agent effectuates expression of functional NOD2 at a level of about 170% of that observed in a human subject of comparable age and body mass index that does not have Crohn's disease. In some embodiments, the agent effectuates expression of functional NOD2 at a level of about 180% of that observed in a human subject of comparable age and body mass index that does not have Crohn's disease. In some embodiments, the agent effectuates expression of functional NOD2 at a level of about 190% of that observed in a human subject of comparable age and body mass index that does not have Crohn's disease. In some embodiments, the agent effectuates expression of functional NOD2 at a level of about 200% of that observed in a human subject of comparable age and body mass index that does not have Crohn's disease.

As used herein, an agent that “increases expression and/or activity of NOD2” is preferably not one that will stimulate functional NOD2 expression in a manner sufficiently excessive to induce pathology. For example, an agent that “increases expression and/or activity of NOD2” is desirably one that recapitulates physiologically normal levels of functional NOD2 expression in a patient (e.g., a human patient having Crohn's disease) that has a NOD2 deficiency.

As used herein, the term “alkyl” refers to monovalent, optionally branched alkyl groups, such as those having from 1 to 6 carbon atoms, or more. This term is exemplified by groups such as methyl, ethyl, n-propyl, isopropyl, n-butyl, isobutyl, tert-butyl, n-hexyl and the like.

As used herein, the term “lower alkyl” refers to alkyl groups having from 1 to 6 carbon atoms.

As used herein, the term “aryl” refers to an unsaturated aromatic carbocyclic group of from 6 to 14 carbon atoms having a single ring (e.g., phenyl) or multiple condensed rings (e.g., naphthyl). Preferred aryl include phenyl, naphthyl, phenanthrenyl and the like.

As used herein, the terms “aralkyl” and “aryl alkyl” are used interchangeably and refer to an alkyl group containing an aryl moiety. Similarly, the terms “aryl lower alkyl” and the like refer to lower alkyl groups containing an aryl moiety.

As used herein, the term “alkyl aryl” refers to alkyl groups having an aryl substituent, including benzyl, phenethyl and the like.

As used herein, the term “heteroaryl” refers to a monocyclic heteroaromatic, or a bicyclic or a tricyclic fused-ring heteroaromatic group. Particular examples of heteroaromatic groups include optionally substituted pyridyl, pyrrolyl, furyl, thienyl, imidazolyl, oxazolyl, isoxazolyl, thiazolyl, isothiazolyl, pyrazolyl, 1,2,3-triazolyl, 1,2,4-triazolyl, 1,2,3-oxadiazolyl, 1,2,4-oxadia-zolyl, 1,2,5-oxadiazolyl, I,3,4-oxadiazolyl,1,3,4-triazinyl, 1,2,3-triazinyl, benzofuryl, [2,3-dihydrojbenzofuryl, isobenzofuryl, benzothienyl, benzotriazolyl, isobenzothienyl, indolyl, isoindolyl, 3H-indolyl, benzimidazolyl, imidazo[I,2-a]pyridyl, benzothiazolyl, benzoxa-zolyl, quinolizinyl, quinazolinyl, pthalazinyl, quinoxalinyl, cinnolinyl, napthyridinyl, pyrido[3,4-b]pyridyl, pyrido[3,2-b]pyridyl, pyrido[4,3-b]pyridyl, quinolyl, isoquinolyl, tetrazolyl, 5,6,7,8-tetrahydroquinolyl, 5,6,7,8-tetrahydroisoquinolyl, purinyl, pteridinyl, carbazolyl, xanthenyl, benzoquinolyl, and the like.

As used herein, the term “alkyl heteroaryl” refers to alkyl groups having a heteroaryl substituent, including 2-furylmethyl, 2-thienylmethyl, 2-(1H-indol-3-yl)ethyl and the like.

As used herein, the term “lower alkenyl” refers to alkenyl groups preferably having from 2 to 6 carbon atoms and having at least 1 or 2 sites of alkenyl unsaturation. Exemplary alkenyl groups are ethenyl (—CH═CH₂), n−2-propenyl (allyl, —CH₂CH═CH₂) and the like.

As used herein, the term “alkenyl aryl” refers to alkenyl groups having an aryl substituent, including 2-phenylvinyl and the like.

As used herein, the term “alkenyl heteroaryl” refers to alkenyl groups having a heteroaryl substituent, including 2-(3-pyridinyl)vinyl and the like.

As used herein, the term “lower alkynyl” refers to alkynyl groups preferably having from 2 to 6 carbon atoms and having at least 1-2 sites of alkynyl unsaturation, preferred alkynyl groups include ethynyl (—C≡CH), propargyl (−CH₂C≡CH), and the like.

As used herein, the term “alkynyl aryl” refers to alkynyl groups having an aryl substituent, including phenylethynyl and the like.

As used herein, the term “alkynyl heteroaryl” refers to alkynyl groups having a heteroaryl substituent, including 2-thienylethynyl and the like.

As used herein, the term “cycloalkyl” refers to a monocyclic cycloalkyl group having from 3 to 8 carbon atoms, such as cyclopropyl, cyclobutyl, cyclopentyl, cyclohexyl, cycloheptyl, cyclooctyl, and the like.

As used herein, the term “lower cycloalkyl” refers to a saturated carbocyclic group of from 3 to 8 carbon atoms having a single ring (e.g., cyclohexyl) or multiple condensed rings (e.g., norbornyl). Preferred cycloalkyl include cyclopentyl, cyclohexyl, norbornyl and the like.

As used herein, the term “heterocycloalkyl” refers to a cycloalkyl group in which one or more ring carbon atoms are replaced with a heteroatom, such as a nitrogen atom, an oxygen atom, a sulfur atom, and the like. Exemplary heterocycloalkyl groups are pyrrolidinyl, piperidinyl, oxopiperidinyl, morpholinyl, piperazinyl, oxopiperazinyl, thiomorpholinyl, azepanyl, diazepanyl, oxazepanyl, thiazepanyl, dioxothiazepanyl, azokanyl, tetrahydrofuranyl, tetrahydropyranyl, and the like.

As used herein, the term “alkyl cycloalkyl” refers to alkyl groups having a cycloalkyl substituent, including cyclohexylmethyl, cyclopentylpropyl, and the like.

As used herein, the term “alkyl heterocycloalkyl” refers to C₁-C₆-alkyl groups having a heterocycloalkyl substituent, including 2-(1-pyrrolidinyl)ethyl, 4-morpholinylmethyl, (1-methyl-4-piperidinyl)methyl and the like.

As used herein, the term “carboxy” refers to the group —C(O)OH.

As used herein, the term “alkyl carboxy” refers to C₁-C₆-alkyl groups having a carboxy substituent, including 2-carboxyethyl and the like.

As used herein, the term “acyl” refers to the group —C(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “acyloxy” refers to the group —OC(O)R, wherein R may be, for example, C₁-C₆-alkyl, aryl, heteroaryl, C₁-C₆-alkyl aryl, or C₁-C₆-alkyl heteroaryl, among other substituents.

As used herein, the term “alkoxy” refers to the group —O—R, wherein R is, for example, an optionally substituted alkyl group, such as an optionally substituted C₁-C₆-alkyl, aryl, heteroaryl, C₁-C₆-alkyl aryl, or C₁-C₆-alkyl heteroaryl, among other substituents. Exemplary alkoxy groups include by way of example, methoxy, ethoxy, phenoxy, and the like.

As used herein, the term “alkoxycarbonyl” refers to the group —C(O)OR, wherein R is, for example, hydrogen, C₁-C₆-alkyl, aryl, heteroaryl, C₁-C₆-alkyl aryl, or C₁-C₆-alkyl heteroaryl, among other possible substituents.

As used herein, the term “alkyl alkoxycarbonyl” refers to alkyl groups having an alkoxycarbonyl substituent, including 2-(benzyloxycarbonyl)ethyl and the like.

As used herein, the term “aminocarbonyl” refers to the group —C(O)NRR′, wherein each of R and R′ may independently be, for example, hydrogen, C₁-C₆-alkyl, aryl, heteroaryl, C₁-C₆-alkyl aryl, or C₁-C₆-alkyl heteroaryl, among other substituents.

As used herein, the term “alkyl aminocarbonyl” refers to alkyl groups having an aminocarbonyl substituent, including 2-(dimethylaminocarbonyl)ethyl and the like.

As used herein, the term “acylamino” refers to the group —NRC(O)R′, wherein each of R and R′ may independently be, for example, hydrogen, C₁-C₆-alkyl, aryl, heteroaryl, C₁-C₆-alkyl aryl, or C₁-C₆-alkyl heteroaryl, among other substituents.

As used herein, the term “alkyl acylamino” refers to alkyl groups having an acylamino substituent, including 2-(propionylamino)ethyl and the like.

As used herein, the term “ureido” refers to the group —NRC(O)NR′R″, wherein each of R, R′, and R″ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary ureido groups further include moieties in which R′ and R″, together with the nitrogen atom to which they are attached, form a 3-8-membered heterocycloalkyl ring.

As used herein, the term “alkyl ureido” refers to alkyl groups having an ureido substituent, including 2-(N′-methylureido)ethyl and the like.

As used herein, the term “amino” refers to the group —NRR′, wherein each of R and R′ may independently be, for example, hydrogen, C₁-C₆-alkyl, aryl, heteroaryl, C₁-C₆-alkyl aryl, C₁-C₆-alkyl heteroaryl, cycloalkyl, or heterocycloalkyl, among other substituents. Exemplary amino groups further include moieties in which R and R′, together with the nitrogen atom to which they are attached, can form a 3-8-membered heterocycloalkyl ring.

As used herein, the term “alkyl amino” refers to alkyl groups having an amino substituent, including 2-(1-pyrrolidinyl)ethyl and the like.

As used herein, the term “ammonium” refers to a positively charged group —N+RR′R″, wherein each of R, R′, and R″ may independently be, for example, C₁-C₆-alkyl, C₁-C₆-alkyl aryl, C₁-C₆-alkyl heteroaryl, cycloalkyl, or heterocycloalkyl, among other substituents. Exemplary ammonium groups further include moieties in which R and R′, together with the nitrogen atom to which they are attached, form a 3-8-membered heterocycloalkyl ring.

As used herein, the term “halogen” refers to fluoro, chloro, bromo and iodo atoms.

As used herein, the term “sulfonyloxy” refers to a group —OSO₂—R wherein R is selected from hydrogen, C₁-C₆-alkyl, C₁-C₆-alkyl substituted with halogens, e.g., an —OSO₂—CF₃ group, aryl, heteroaryl, C₁-C₆-alkyl aryl, and C₁-C₆-alkyl heteroaryl.

As used herein, the term “alkyl sulfonyloxy” refers to alkyl groups having a sulfonyloxy substituent, including 2-(methylsulfonyloxy)ethyl and the like.

As used herein, the term “sulfonyl” refers to group “—SO₂—R” wherein R is selected from hydrogen, aryl, heteroaryl, C₁-C₆-alkyl, C₁-C₆-alkyl substituted with halogens, e.g., an —SO₂—CF₃ group, C₁-C₆-alkyl aryl or C₁-C₆-alkyl heteroaryl.

As used herein, the term “alkyl sulfonyl” refers to alkyl groups having a sulfonyl substituent, including 2-(methylsulfonyl)ethyl and the like.

As used herein, the term “sulfinyl” refers to a group “—S(O)—R” wherein R is selected from hydrogen, C₁-C₆-alkyl, C₁-C₆-alkyl substituted with halogens, e.g., a —SO—CF₃ group, aryl, heteroaryl, C₁-C₆-alkyl aryl or C₁-C₆-alkyl heteroaryl.

As used herein, the term “alkyl sulfinyl” refers to C₁-C₅-alkyl groups having a sulfinyl substituent, including 2-(methylsulfinyl)ethyl and the like.

As used herein, the term “sulfanyl” refers to groups —S—R, wherein R is, for example, alkyl, aryl, heteroaryl, C₁-C₆-alkyl aryl, or C₁-C₆-alkyl heteroaryl, among other substituents. Exemplary sulfanyl groups are methylsulfanyl, ethylsulfanyl, and the like.

As used herein, the term “alkyl sulfanyl” refers to alkyl groups having a sulfanyl substituent, including 2-(ethylsulfanyl)ethyl and the like.

As used herein, the term “sulfonylamino” refers to a group —NRSO₂—R′, wherein each of R and R′ may independently be hydrogen, C₁-C₆-alkyl, aryl, heteroaryl, C₁-C₆-alkyl aryl, or C₁-C₆-alkyl heteroaryl, among other substituents.

As used herein, the term “alkyl sulfonylamino” refers to alkyl groups having a sulfonylamino substituent, including 2-(ethylsulfonylamino)ethyl and the like.

Unless otherwise constrained by the definition of the individual substituent, the above set out groups, like “alkyl”, “alkenyl”, “alkynyl”, “aryl” and “heteroaryl” etc. groups can optionally be substituted, for example, with one or more substituents, as valency permits, such as a substituent selected from alkyl (e.g., C₁-C₆-alkyl), alkenyl (e.g., C₂-C₆-alkenyl), alkynyl (e.g., C₂-C₆-alkynyl), cycloalkyl, heterocycloalkyl, alkyl aryl (e.g., C₁-C₆-alkyl aryl), alkyl heteroaryl (e.g., C₁-C₆-alkyl heteroaryl, alkyl cycloalkyl (e.g., C₁-C₆-alkyl cycloalkyl), alkyl heterocycloalkyl (e.g., C₁-C₆-alkyl heterocycloalkyl), amino, ammonium, acyl, acyloxy, acylamino, aminocarbonyl, alkoxycarbonyl, ureido, aryl, heteroaryl, sulfinyl, sulfonyl, alkoxy, sulfanyl, halogen, carboxy, trihalomethyl, cyano, hydroxy, mercapto, nitro, and the like. In some embodiments, the substitution is one in which neighboring substituents have undergone ring closure, such as situations in which vicinal functional substituents are involved, thus forming, e.g., lactams, lactones, cyclic anhydrides, acetals, thioacetals, and aminals, among others.

As used herein, the term “optionally fused” refers to a cyclic chemical group that may be fused with a ring system, such as cycloalkyl, heterocycloalkyl, aryl, or heteroaryl. Exemplary ring systems that may be fused to an optionally fused chemical group include, e.g., indolyl, isoindolyl, benzofuranyl, isobenzofuranyl, benzothiophenyl, benzoxazolyl, benzothiazolyl, benzoisoxazolyl, benzoisothiazolyl, indazolyl, benzimidazolyl, quinolinyl, isoquinolinyl, phthalazinyl, quinoxalinyl, quinazolinyl, cinnolinyl, indolizinyl, naphthyridinyl, pteridinyl, indanyl, naphtyl, 1,2,3,4-tetrahydronaphthyl, indolinyl, isoindolinyl, 2,3,4,5-tetrahydrobenzo[b]oxepinyl, 6,7,8,9-tetrahydro-5H-benzocycloheptenyl, chromanyl, and the like.

As used herein, the term “pharmaceutically acceptable salt” refers to a salt, such as a salt of a compound described herein, that retains the desired biological activity of the non-ionized parent compound from which the salt is formed. Examples of such salts include, but are not restricted to acid addition salts formed with inorganic acids (e.g., hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disulfonic acid, and poly-galacturonic acid. The compounds can also be administered as pharmaceutically acceptable quaternary salts, such as quaternary ammonium salts of the formula —NR,R′,R″ ⁺Z⁻, wherein each of R, R′, and R″ may independently be, for example, hydrogen, alkyl, benzyl, C₁-C₆-alkyl, C₂-C₆-alkenyl, C₂-C₆-alkynyl, C₁-C₆-alkyl aryl, C₁-C₆-alkyl heteroaryl, cycloalkyl, heterocycloalkyl, or the like, and Z is a counterion, such as chloride, bromide, iodide, —O-alkyl, toluenesulfonate, methyl sulfonate, sulfonate, phosphate, carboxylate (such as benzoate, succinate, acetate, glycolate, maleate, malate, fumarate, citrate, tartrate, ascorbate, cinnamoate, mandeloate, and diphenylacetate), or the like.

As used herein, for example, in the context of a protein kinase C (PKC) inhibitor, such as staurosporine, the term “variant” refers to an agent containing one or more modifications relative to a reference agent and that (i) retains a functional property of the reference agent (e.g., the ability to inhibit PKC activity) and/or (ii) is converted within a cell (e.g., a cell of a type described herein, such as a CD34+ cell) into the reference agent. In the context of small molecule PKC inhibitors, such as staurosporine, structural variants of a reference compound include those that differ from the reference compound by the inclusion and/or location of one or more substituents, as well as variants that are isomers of a reference compound, such as structural isomers (e.g., regioisomers) or stereoisomers (e.g., enantiomers or diastereomers), as well as prodrugs of a reference compound. In the context of an interfering RNA molecule, a variant may contain one or more nucleic acid substitutions relative to a parent interfering RNA molecule.

The structural compositions described herein also include the tautomers, geometrical isomers (e.g., E/Z isomers and cis/trans isomers), enantiomers, diastereomers, and racemic forms, as well as pharmaceutically acceptable salts thereof. Such salts include, e.g., acid addition salts formed with pharmaceutically acceptable acids like hydrochloride, hydrobromide, sulfate or bisulfate, phosphate or hydrogen phosphate, acetate, benzoate, succinate, fumarate, maleate, lactate, citrate, tartrate, gluconate, methanesulfonate, benzenesulfonate, and para-toluenesulfonate salts.

As used herein, chemical structural formulas that do not depict the stereochemical configuration of a compound having one or more stereocenters will be interpreted as encompassing any one of the stereoisomers of the indicated compound, or a mixture of one or more such stereoisomers (e.g., any one of the enantiomers or diastereomers of the indicated compound, or a mixture of the enantiomers (e.g., a racemic mixture) or a mixture of the diastereomers). As used herein, chemical structural formulas that do specifically depict the stereochemical configuration of a compound having one or more stereocenters will be interpreted as referring to the substantially pure form of the particular stereoisomer shown. “Substantially pure” forms refer to compounds having a purity of greater than 85%, such as a purity of from 85% to 99%, 85% to 99.9%, 85% to 99.99%, or 85% to 100%, such as a purity of 85%, 86%, 87%, 88%, 89%, 90%, 91%, 92%, 93%, 94%, 95%, 96%, 97%, 98%, 99%, 99.9%, 99.99%, 99.999%, or 100%, as assessed, for example, using chromatography and nuclear magnetic resonance techniques known in the art.

### BRIEF DESCRIPTION OF THE FIGURES

FIG. 1 is a schematic demonstrating an exemplary procedure that may be used to recapitulate functional NOD2 expression at a genetic locus that is near or within an endogenous gene encoding a defective NOD2 protein. A genetic locus in a target cell, such as an autologous cell obtained from a patient suffering from Crohn's disease, may be edited at a site near or within the gene encoding endogenous NOD2. The gene encoding endogenous NOD2 may have a mutation causing a NOD2 defect. To edit the target cell genome at this site, the cell may be provided a nuclease, such as a CRISPR-associated protein, along with a guide RNA (g RNA) and a template nucleic acid that encodes functional NOD2. The gRNA may direct the nuclease to the desired site within the target cell by base pair hybridization. The nuclease may then catalyze a single-strand break or double-strand break at the desired site, at which point the template nucleic acid encoding functional NOD2 may insert into the target cell genome at the desired site. The template nucleic acid encoding functional NOD2 may insert at a site that is operably joined to the endogenous NOD2 promoter, resulting in recapitulation of functional NOD2 protein expression.

FIGS. 2A-2C are graphs showing that NOD2 activation effectuates robust inflammatory cytokine release by human monocytes. THP-1 human monocytic cells were pre-stimulated for 18 hours with 10 ng/mL PMA (FIG. 2A), 10 ng/mL LPS (FIG. 2B), or 5 ng/mL TNFα (FIG. 2C) followed by treatment with MDP to induce NOD2 signaling. NOD2-dependent cytokine production was assayed in cell supernatants after 18-24 hours by flow based cytometric bead array analysis and ELISA. Data shown is representative of at least 3 independent experiments. PMA: phorbol 12-myristate 13-acetate; LPS: lipopolysaccharide; MDP: muramyl dipeptide. THP-1: ATCC No. TIB-202.

FIG. 3 is a graph showing that NOD2 activation effectuates robust inflammatory cytokine release by peripheral blood CD14+ monocytes in the absence of priming. Peripheral blood CD14+ monocytes isolated from healthy donors were treated with MDP to induce NOD2 signaling. NOD2-dependent cytokine production was assayed in cell supernatants after 18-24 hours by flow based cytometric bead array analysis. Data shows fold increase in cytokine production relative to unstimulated cells and is representative of at least 3 independent experiments.

FIGS. 4A and 4B are graphs demonstrating that wild-type murine tissue-isolated and bone marrow-derived monocytes show a characteristic proinflammatory response to NOD2 activation. Primary murine peritoneal macrophages (CD11b+) were primed overnight by LPS treatment, followed by stimulation of NOD2 signaling by MDP treatment (FIG. 4A). Murine bone marrow-derived macrophages, generated by ex vivo culture in GM-CSF, were primed by overnight treatment with LPS (10 ng/mL), followed by stimulation of NOD2 signaling by MDP treatment (FIG. 4B). NOD2 stimulation resulted in release of active/processed IL-1β detected by ELISA. Data shown is representative of at least 3 independent experiments, where UT indicates untreated cells, isolated from wild-type C57BL6 mice.

FIG. 5 is a graph showing that NOD2 disruption impairs the THP-1 monocyte inflammatory cytokine response to MDP. Several NOD2-mutant THP-1 clonal cell lines were generated using CRISPR-Cas9 to model NOD2-deficiency in Crohn's Disease. Wildtype (WT), several exon-2 and exon-8 targeted NOD2 knock out clones (KO), and THP-1 cells undergoing mock CRISPR-Cas9 NOD2 disruption (Mock), were primed with LPS overnight, followed by stimulation with MDP (10 μg/mL). The relative increase in IL-8 release is expressed as fold change relative to untreated cells (UT), detected by ELISA analysis of cell supernatants. NOD2 KO THP-1 clones show an inability to generate a proinflammatory cytokine response to MDP stimulation. Data shown is representative of at least 3 independent experiments. Statistical significance paired t-test untreated vs MDP *p<0.001.

FIG. 6 is a graph showing that NOD2 disruption by CRISPR-Cas9 gene editing impairs CD34+ HSC-derived myeloid inflammatory cytokine response to MDP. Peripheral blood-derived CD34+ cells isolated from healthy donors were subject to targeted disruption of NOD2 by CRISPR-Cas9 gene editing (RNP+guideRNA nucleofection). Gene edited cells, NOD2 KO cells, and MOCK edited cells (receiving RNP only) were then cultured for 14 days in the presence of cytokines to promote differentiation towards monocyte/macrophage lineage committed cells. Cell cultures were then stimulated with MDP (0-100 μg/mL) for 18-24 hours and cell supernatants were assayed for IL-8 cytokine release by ELISA. Gene editing NOD2 KO efficiency (85-90%) in CD34+ cells was confirmed by T7 endonuclease assay and Inference of CRISPR Edits (ICE) analysis (not shown). Data shown is representative of 2 independent experiments.

FIGS. 7A-7C are graphs showing that NOD2−/− mice have an impaired macrophage inflammatory cytokine responses to MDP. WT and NOD2−/− murine bone marrow-derived macrophages (FIGS. 7A and 7B) and monocytes (FIG. 7C) generated by ex vivo culture in GM-CSF or M-CSF, respectively, were primed by overnight treatment with LPS (1 ng/mL), followed by stimulation of NOD2 signaling by MDP treatment. NOD2 stimulation resulted in release of IL-6 (FIG. 7A), TNFα (FIG. 7B), and active/processed IL-1β (FIG. 7C) by WT-derived cells, but was absent in NOD2−/− cells, as detected by flow cytometric bead array analysis or ELISA. Data shown is representative of at least 3 independent experiments and is expressed as fold change in levels of cytokine release upon MDP treatment relative to untreated cells. Statistical significance t-test NOD2−/− vs WT *p<0.001.

FIGS. 8A-8D show the design and validation of lentiviral vectors to restore functional NOD2 expression under the control of various promoters to regulate gene expression, as well as the use of codon-optimized sequences to deliver higher transgene expression. FIG. 8A is a schematic showing some of the lentiviral constructs generated to restore functional NOD2 gene expression, under constitutive (EF1α/EFS), myeloid lineage specific (CD11b) or endogenous NOD2 (NOD2p) promoter control, of codon optimized (coNOD2) or WT NOD2 protein, or an irrelevant protein (GFP). THP-1 monocytes were transduced with lentiviral vectors (multiplicity of infection (moi) 10) and relative gene expression of WT NOD2 (FIG. 8B) and coNOD2 (FIG. 8C) were detected by transgene-specific RT-PCR analysis after 4 days (relative to untransduced cells). Inset data shows quantification of the transgene vector copy number (VCN) detected by analysis of genomic DNA. Flow cytometry dotplots showing staining of human NOD2 protein in Untreated (UT) and transduced (LV-NOD2, LV-coNOD2) HT29 cells. Data shown is representative of 3 independent experiments (FIG. 8D).

FIG. 9 is a graph showing that lentiviral transduction of murine bone marrow HSC restores functional NOD2 expression in NOD2−/− monocytes. Bone marrow lineage negative HSC isolated from WT or NOD2−/− mice were transduced with a lentiviral vector encoding NOD2 (LV-NOD2, EFS promoter). Murine bone marrow-derived macrophages were then generated by ex vivo culture in GM-CSF. Cells were primed by overnight treatment with LPS (1 ng/mL), followed by stimulation of NOD2 signaling by MDP (10 μg/mL) treatment. NOD2-mediated IL-6 production was detected in cell supernatants by ELISA after 18-24 hours. IL-6 production is expressed as fold increase in cytokine levels relative to untreated cells (UT). Data shown is representative of 2 independent experiments. Statistical significance t-test Untransduced vs Transduced *p<0.001.

FIGS. 10A-10D show that lentiviral transduction of NOD2-deficient THP-1 cells can restore human monocyte inflammatory responses to MDP. THP-1 WT and CRISPR-Cas9 gene edited clones (NOD2KO or mock edited) were transduced with LV-coNOD2 vector (moi 10). Three days after transduction, THP-1 cells were primed with LPS and then treated with MDP (10 μg/mL) to stimulate NOD2 activity. (FIG. 10A) Lentiviral transduction of NOD2 KO THP-1 clones resulted in restoration of NOD2-dependent IL-8 cytokine release detected by ELISA. (FIG. 10B) Lentiviral transduction efficiency of THP-1 cells was confirmed by assessing their transduction using a GFP reporter-LV construct; flow cytometry dotplots show representative transduction efficiency achieved at LV moi 10. (FIG. 100) NOD2 gene expression was confirmed by RT-PCR analysis of transduced cells (relative to untransduced cells). (FIG. 10D) Quantification of the transgene vector copy number (mean VCN) detected by analysis of genomic DNA in transduced THP-1 cells. Data shown is representative of 2-3 independent experiments. Statistical significance paired t-test Transduced vs Untransduced *p<0.001.

FIGS. 11A-11D are graphs showing that lentiviral transduction of NOD2-deficient THP-1 cells can restore human monocyte inflammatory responses to MDP. THP-1 WT and CRISPR-Cas9 gene edited clones (KO exon 8 clones 1 & 2, or mock edited) were transduced with lentiviral vectors (moi 10) encoding wild type (NOD2) or codon-optimized NOD2 (coNOD2) sequences, or an irrelevant encoded protein, GFP. Three days after transduction, THP-1 cells were primed with LPS and then treated with MDP (10 μg/mL) to stimulate NOD2 activity. (FIG. 11A) MDP exposure and priming with LPS results in elevated IL-8 release in wild-type and mock-treated cells. Samples from left to right along the horizontal axis denote (i) untransduced cells, (ii) untransduced cells treated with MDP, and (iii) untransduced cells primed with LPS. (FIG. 11B) MDP exposure results in a robust IL-8 release response in cells transduced with a lentiviral vector containing a NOD2 transgene under the control of an EFS promoter. Samples from left to right along the horizontal axis denote (i) untransduced cells, (ii) untransduced cells treated with MDP, (iii) cells transduced with a lentiviral vector containing a wild-type NOD2 transgene under the control of an EFS promoter, in the absence of MDP, (iv) cells transduced with a lentiviral vector containing a wild-type NOD2 transgene under the control of an EFS promoter, in the presence of MDP, (v) cells transduced with a lentiviral vector containing a codon-optimized NOD2 transgene under the control of an EFS promoter, in the absence of MDP, (vi) cells transduced with a lentiviral vector containing a codon-optimized NOD2 transgene under the control of an EFS promoter, in the presence of MDP, (vii) cells transduced with a lentiviral vector containing a wild-type GFP transgene under the control of a CD11b promoter, in the absence of MDP, and (viii) cells transduced with a lentiviral vector containing a wild-type GFP transgene under the control of a CD11b promoter, in the presence of MDP. (FIG. 11C) MDP exposure results in a robust IL-8 release response in cells transduced with a lentiviral vector containing a NOD2 transgene under the control of a CD11b promoter. Samples from left to right along the horizontal axis denote (i) untransduced cells, (ii) untransduced cells treated with MDP, (iii) cells transduced with a lentiviral vector containing a wild-type NOD2 transgene under the control of a CD11b promoter, in the absence of MDP, (iv) cells transduced with a lentiviral vector containing a wild-type NOD2 transgene under the control of a CD11b promoter, in the presence of MDP, (v) cells transduced with a lentiviral vector containing a codon-optimized NOD2 transgene under the control of a CD11b promoter, in the absence of MDP, (vi) cells transduced with a lentiviral vector containing a codon-optimized NOD2 transgene under the control of a CD11b promoter, in the presence of MDP, (vii) cells transduced with a lentiviral vector containing a wild-type GFP transgene under the control of a CD11b promoter, in the absence of MDP, and (viii) cells transduced with a lentiviral vector containing a wild-type GFP transgene under the control of a CD11b promoter, in the presence of MDP. (FIG. 11D) MDP exposure results in a robust IL-8 release response in cells transduced with a lentiviral vector containing a NOD2 transgene under the control of an endogenous NOD2 promoter. Samples from left to right along the horizontal axis denote (i) untransduced cells, (ii) untransduced cells treated with MDP, (iii) cells transduced with a lentiviral vector containing a wild-type NOD2 transgene under the control of a wild-type endogenous NOD2 promoter, in the absence of MDP, (iv) cells transduced with a lentiviral vector containing a wild-type NOD2 transgene under the control of a wild-type endogenous NOD2 promoter, in the presence of MDP, (v) cells transduced with a lentiviral vector containing a codon-optimized NOD2 transgene under the control of a wild-type endogenous NOD2 promoter, in the absence of MDP, (vi) cells transduced with a lentiviral vector containing a codon-optimized NOD2 transgene under the control of a wild-type endogenous NOD2 promoter, in the presence of MDP, (vii) cells transduced with a lentiviral vector containing a wild-type GFP transgene under the control of a CD11b promoter, in the absence of MDP, and (viii) cells transduced with a lentiviral vector containing a wild-type GFP transgene under the control of a CD11b promoter, in the presence of MDP.

FIG. 12 is a graph illustrating NOD2 gene transfer by lentiviral transduction of peripheral blood-derived CD34+ cells. CD34+ cells isolated from mobilized peripheral blood of healthy human volunteers were transduced with lentiviral vectors (moi 10 & 50) generated to transfer WT or codon optimized NOD2 under the control of a myeloid lineage-specific promoter (CD11b promoter) or a constitutive promoter (EFS promoter). Data shows quantification of the transgene vector copy number (mean VCN) detected by analysis of genomic DNA isolated from myeloid cell cultures 14 days post-transduction and is representative of 3 independent experiments.

FIGS. 13A and 13B are graphs showing that NOD2 gene transfer by lentiviral transduction of NOD2-KO peripheral blood-derived CD34+ cells can partially restore MDP detection by differentiated CD34+ cell cultures. CD34+ cells isolated from mobilized peripheral blood of healthy human volunteers were firstly subject to gene editing by CRISPR-Cas9 to disrupt NOD2 (NOD2KO or mock) and then transduced with LV-coNOD2 vectors. CD34+ cells were then differentiated in vitro for 2 weeks (final cultures composed of 15-30% CD11b+CD14+ cells) after which cultures were treated with MDP to stimulate NOD2 activity. Lentiviral transduction of NOD2 KO cells resulted in partial restoration of NOD2-dependent (FIG. 13A) IL-8 and (FIG. 13B) TNFα cytokine release upon MDP stimulation (1 μg/mL) detected by ELISA. CD34+ cells were transduced with lentiviral vectors generated to transfer WT NOD2 or codon optimized NOD2 under the control of a myeloid lineage-specific promoter (CD11b promoter) or a constitutive promoter (EFS promoter).

FIG. 14 shows a pair of flow cytometry contour plots illustrating the ability of gene editing to effectuate targeted GFP insertion into the NOD2 locus in CD34+ HSCs. A GFP reporter sequence was used to validate a gene editing strategy for targeted insertion of a payload donor template into exon2 of the NOD2 gene locus. Gene editing of peripheral blood derived CD34+ cells was performed using CRISPR-Cas9 RNP nucleofection. Donor payload delivery was achieved using a template sequence delivered by an AAV6 vector. Particularly, the donor payload included a transgene encoding GFP under the control of the EFS promoter. The efficiency of homology-directed repair was confirmed by flow cytometry detection of GFP+ cells in myeloid differentiated cell cultures. Data shown is representative of 2 independent experiments. Targeting to the NOD2 locus was confirmed by an ‘In-Out’ PCR approach, in which one primer is located in the targeted genomic locus outside the homology arm and the other primer is located inside the transgene cassette (data not shown).

### DETAILED DESCRIPTION

The present disclosure provides compositions and methods for treating or preventing Crohn's disease. The compositions and methods described herein may be used, for example, to treat a patient, such as an adult human patient, that is suffering from Crohn's disease, as well as to prophylactically treat a patient at risk of developing Crohn's disease. Patients may be treated, for example, by providing to the patients one or more agents that elevate the expression and/or activity of functional Nucleotide-binding oligomerization domain-containing protein 2 (NOD2), such as a population of cells (e.g., a population of pluripotent cells, such as hematopoietic stem cells) that express functional NOD2. Without being limited by mechanism, the provision of such agents may treat an underlying cause of the disease and reverse its pathophysiology. Thus, using the compositions and methods described herein, a patient may not only be treated in a manner that alleviates one or more symptoms associated with Crohn's disease, but also in a curative fashion or preventative fashion.

### NOD2 Signal Transduction

NOD2 is an intracellular pattern recognition receptor (PRR), which recognizes bacterial pathogens and initiates an immune response accordingly. As a PRR, NOD2 recognizes bacterial lipopolysaccharide (LPS), muramyldipeptide (MDP), and other pathogen-associated molecular patterns (PAMPs). NOD2 is a 110 kDa cytoplasmic protein belonging to the Nod-like receptor (NLR) family. Its expression is largely restricted to monocytes and other antigen-presenting cells (APCs). Proteins in the NLR family generally contain a nucleotide-binding oligomerization domain (NOD) responsible for coordinating protein oligomerization. NOD2 also contains a C-terminal leucine-rich repeat (LRR) domain with 11 LRR repeats. The LRR domain coordinates ligand binding and other protein-protein interactions. NOD2 also contains two N-terminal caspase associated recruitment domains (CARDs). The CARDs recruit proteins to promote apoptosis. The NOD2 CARDs are also capable of activating NFκB signaling via a homophilic CARD-CARD interaction with RICK, a serine-threonine kinase. RICK then associates with IKKγ to promote IKK-dependent activation of NFκB.

Using the compositions and methods of the disclosure, an agent that increases NOD2 activity and/or expression, such as a cell (e.g., a CD34+ cell or other pluripotent cell described herein) that expresses NOD2, can be administered to a patient suffering from Crohn's disease (e.g., a patient having a defect in NOD2 expression) so as to promote the following beneficial characteristics:

(i) restoration of physiologically normal levels of NOD2 expression;

(ii) an increase in MDP detection;

(iii) an increase in NFκB signal transduction; and/or

(iv) an augmented immune response against pathogenic microbes.

Without being limited by mechanism, the section that follows describes how agents that increase the NOD2 activity and/or expression and effectuate one or more, or all, of the beneficial phenotypes described above can be used to treat Crohn's disease.

### Crohn's Disease

### Etiology and NOD2 Restoration Therapy

Crohn's disease is an autoinflammatory diseases that can be caused by defective NOD2 activity. This aberration in NOD2 activity can be triggered by mutations clustered in the nucleotide-binding oligomerization domain (NBD) of NOD2. Such mutations include R702W, G908R, L1007fs.

NOD2 is usually maintained in an inactive, autoinhibited conformation in the cell by way of interactions between the NACHT and leucine-rich repeat (LRR) domains, as well as interaction with cellular chaperones. NOD2 is activated upon recognition of MDP, through direct peptide interaction with the LRR. NOD2 and MDP association induces a conformational change based activation of the NOD2 protein. However, mutations in NOD2, such as those described above, prevent interaction and sensing of MDP, and subsequent activation of the NOD2 protein.

Using the compositions and methods of the disclosure, a patient, such as a human patient suffering from Crohn's disease, may be administered an agent that expresses a functional NOD2 protein that does not contain an activity-disrupting mutation. Exemplary agents that achieve this effect are pluripotent cells, such as hematopoietic stem cells and hematopoietic progenitor cells, that express functional NOD2. The sections that follow describe exemplary procedures for producing such agents, as well as how such agents may be used to treat a patient suffering from Crohn's disease.

### Diagnosis

A patient (e.g., a human patient) can be diagnosed as having Crohn's disease in a variety of ways. Genetic testing offers one avenue by which a patient may be diagnosed as having (or at risk of developing) Crohn's disease. For example, a genetic analysis can be used to determine whether a patient has a loss-of-function mutation in an endogenous gene encoding NOD2, such as a mutation in a NOD2 gene selected from the group consisting of R702W, G908R, and L1007fs. Exemplary genetic tests that can be used to determine whether a patient has such a mutation include polymerase chain reaction (PCR) methods known in the art and described herein, among others.

Clinically, Crohn's disease may be detected, for example, by way of a blood test. In this setting, Crohn's disease may manifest as anemia, a condition characterized by an insufficiency of red blood cells to deliver oxygen to tissues. Crohn's disease may also manifest in the form of infection (e.g., a bacterial infection), which can be detected in blood by identifying bacterial nucleic acids, for example, using molecular biology techniques known in the art, such as PCR-based methods, among others.

Another indicator of Crohn's disease is the presence of occult blood in a patient's stool. This can readily be assessed upon analysis of a stool sample obtained from the patient.

Crohn's disease may also be detected by way of a colonoscopy. This procedure allows inspection of the entire colon and the end of the ileum. Clusters of inflammatory granulomas, if present, may confirm a diagnosis of Crohn's disease.

Another useful procedure for diagnosing a patient as having Crohn's disease is computerized tomography (CT). A CT scan may be used to inspect the entire bowel as well as at tissues exterior to the bowel. This technique allows the detection of complications associate with Crohn's disease, including abscesses, fistulas, and intestinal blockages.

A further procedure that can be used to facilitate a diagnosis of Crohn's disease is magnetic resonance imaging (MRI). MRI is particularly useful for evaluating a fistula proximal to the anus (e.g., detectable by way of a pelvic MRI) or the small intestine (e.g., detectable by way of MR enterography). Either or both may be indicative of Crohn's disease.

Another technique that may be used to detect Crohn's disease in a patient is capsule endoscopy. In this procedure, the patient swallows a capsule containing a microscale camera, which visualizes the small intestine. The images thus obtained may be inspected for signs of infection, which may be indicative of Crohn's disease.

In yet another technique, one may be diagnosed as having Crohn's disease by way of balloon-assisted enteroscopy. In this procedure, a scope is used to visualize further into the small bowel, particularly in regions not accessible to standard endoscopes. This technique is often useful when a capsule endoscopy reveals abnormalities, but the diagnosis is still in question.

### Prevention

Using the compositions and methods described herein, a subject (e.g., a human subject) may be administered one or more agents that increase activity and/or expression of functional NOD2 (e.g., to within physiologically normal levels) so as to prevent the onset of Crohn's disease. The subject may be one that is at risk of developing Crohn's disease, but has not yet presented with an observable symptom of the disease. For example, the subject may be one that has a loss-of-function mutation in an endogenous gene encoding NOD2, such as a mutation in a NOD2 gene selected from the group consisting of R702W, G908R, and L1007fs. As described above, a subject can be identified as having such a mutation using standard molecular biology techniques known in the art and described herein, including PCR-based methodologies, among others.

### Methods of Producing Functional NOD2-Expressing Cells by Viral Transduction

### Transduction Using a Protein Kinase C Modulator

A variety of agents can be used to reduce PKC activity and/or expression. Without being limited by mechanism, such agents can augment viral transduction by stimulating Akt signal transduction and/or maintaining cofilin in a dephosphorylated state, thereby promoting actin depolymerization. This actin depolymerization event may serve to remove a physical barrier that hinders entry of a viral vector into the nucleus of a target cell.

Staurosporine and Variants Thereof

In some embodiments, the substance that reduces activity and/or expression of PKC is a PKC inhibitor. The PKC inhibitor may be staurosporine or a variant thereof. For example, the PKC inhibitor may be a compound represented by formula (I)

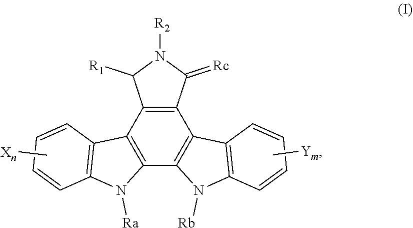

wherein R₁ is H, OH, optionally substituted alkoxy, optionally substituted acyloxy, optionally substituted amino, optionally substituted alkylamino, optionally substituted amido, halogen, optionally substituted C₁₋₆ alkyl, optionally substituted C₂₋₆ alkenyl, optionally substituted C₂₋₆ alkynyl, optionally substituted acyl, optionally substituted alkoxycarbonyl, oxo, thiocarbonyl, optionally substituted carboxy, or ureido;

R₂ is H, optionally substituted C₁₋₆ alkyl, optionally substituted C₂₋₆ alkenyl, optionally substituted C₂₋₆ alkynyl, or optionally substituted acyl;

Rₐ and R_{b} are each, independently, H, optionally substituted C₁₋₆ alkyl, optionally substituted C₂₋₆ alkenyl, or optionally substituted C₂₋₆ alkynyl, optionally substituted and optionally fused aryl, optionally substituted and optionally fused heteroaryl, optionally substituted and optionally fused cycloalkyl, or optionally substituted and optionally fused heterocycloalkyl, or Rₐ and R_{b}, together with the atoms to which they are bound, are joined to form an optionally substituted and optionally fused heterocycloalkyl ring;

R_{c} is O, NR_{d}, or S;

R_{d} is H, optionally substituted C₁₋₆ alkyl, optionally substituted C₂₋₆ alkenyl, or optionally substituted C₂₋₆ alkynyl;

each X is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;

each Y is, independently, halogen, optionally substituted haloalkyl, cyano, optionally substituted amino, hydroxyl, thiol, optionally substituted alkoxy, optionally substituted alkylthio, optionally substituted acyloxy, optionally substituted alkoxycarbonyl, optionally substituted carboxy, ureido, optionally substituted alkyl sulfonyl, optionally substituted aryl sulfonyl, optionally substituted heteroaryl sulfonyl, optionally substituted cycloalkyl sulfonyl, optionally substituted heterocycloalkyl sulfonyl, optionally substituted alkyl sulfanyl, optionally substituted aryl sulfanyl, optionally substituted heteroaryl sulfanyl, optionally substituted cycloalkyl sulfanyl, optionally substituted heterocycloalkyl sulfanyl, optionally substituted alkyl sulfinyl, optionally substituted aryl sulfinyl, optionally substituted heteroaryl sulfinyl, optionally substituted cycloalkyl sulfinyl, optionally substituted heterocycloalkyl sulfinyl, optionally substituted alkyl, optionally substituted alkenyl, optionally substituted alkynyl, optionally substituted and optionally fused aryl, optionally substituted and optionally fused heteroaryl, optionally substituted and optionally fused cycloalkyl, or optionally substituted and optionally fused heterocycloalkyl;

[symbol] represents a bond that is optionally present;

n is an integer from 0-4; and

m is an integer from 0-4;

or a salt thereof.

Interfering RNA

Exemplary PKC modulating agents that may be used in conjunction with the compositions and methods of the disclosure include interfering RNA molecules, such as short interfering RNA (siRNA), short hairpin RNA (shRNA), and/or micro RNA (miRNA), that diminish PKC gene expression. Methods for producing interfering RNA molecules are known in the art and are described in detail, for example, in WO 2004/044136 and U.S. Pat. No. 9,150,605, the disclosures of each of which are incorporated herein by reference in their entirety.

### Transduction Using an HDAC Inhibitor

A variety of agents can be used to inhibit histone deacetylases in order to increase the expression of a transgene during viral transduction. Without wishing to be bound by theory, reduced transgene expression from viral vectors may be caused by epigenetic silencing of vector genomes carried out by histone deacetylates. Hydroxamic acids represent a particularly robust class of HDAC inhibitors that inhibit these enzymes by virtue of hydroxamate functionality that binds cationic zinc within the active sites of these enzymes. Exemplary inhibitors include trichostatin A, as well as Vorinostat (N-hydroxy-N′-phenyl-octanediamide, described in Marks et al., Nature Biotechnology 25, 84 to 90 (2007); Stenger, Community Oncology 4, 384-386 (2007), the disclosures of which are incorporated by reference herein). Other HDAC inhibitors include Panobinostat, described in Drugs of the Future 32(4): 315-322 (2007), the disclosure of which is incorporated herein by reference.

Additional examples of hydroxamic acid inhibitors of histone deacetylases include the compounds shown below, described in Bertrand, European Journal of Medicinal Chemistry 45:2095-2116 (2010), the disclosure of which is incorporated herein by reference.

Other HDAC inhibitors that do not contain a hydroxamate substituent have also been developed, including Valproic acid (Gottlicher, et al., EMBO J. 20(24): 6969-6978 (2001) and Mocetinostat (N-(2-Aminophenyl)-4-[[(4-pyridin-3-ylpyrimidin-2-yl)amino]methyl]benzamide, described in Balasubramanian et al., Cancer Letters 280: 211-221 (2009)), the disclosure of each of which is incorporated herein by reference. Other small molecule inhibitors that exploit chemical functionality distinct from a hydroxamate include those described in Bertrand, European Journal of Medicinal Chemistry 45:2095-2116 (2010), the disclosure of which is incorporated herein by reference.

Additional examples of chemical modulators of histone acetylation useful with the compositions and methods of the invention include modulators of HDAC1, HDAC2, HDAC3, HDAC4, HDAC5, HDAC6, HDAC7, HDAC8, HDAC9, HDAC10, Sirt1, Sirt2, and/or HAT, such as butyrylhydroxamic acid, M344, LAQ824 (Dacinostat), AR-42, Belinostat (PXD101), CUDC-101, Scriptaid, Sodium Phenylbutyrate, Tasquinimod, Quisinostat (JNJ-26481585), Pracinostat (SB939), CUDC-907, Entinostat (MS-275), Mocetinostat (MGCD0103), Tubastatin A HCl, PCI-34051, Droxinostat, PCI-24781 (Abexinostat), RGFP966, Rocilinostat (ACY-1215), CI994 (Tacedinaline), Tubacin, RG2833 (RGFP109), Resminostat, Tubastatin A, BRD73954, BG45, 4SC-202, CAY10603, LMK-235, Nexturastat A, TMP269, HPOB, Cambinol, and Anacardic Acid.

In some particular embodiments, the HDAC inhibitor is Scriptaid.

### Transduction Using a Cyclosporine

In some embodiments, therapeutic cells of the disclosure are produced by transducing the cells in the presence of a cyclosporine, such as cyclosporine A (CsA) or cyclosporine H (CsH).

In some embodiments, the concentration of the cyclosporine, when contacted with the cell, is from about 1 μM to about 10 μM (e.g., about 1 μM, 1.1 μM, 1.2 μM, 1.3 μM, 1.4 μM, 1.5 μM, 1.6 μM, 1.7 μM, 1.8 μM, 1.9 μM, 2 μM, 2.1 μM, 2.2 μM, 2.3 μM, 2.4 μM, 2.5 μM, 2.6 μM, 2.7 μM, 2.8 μM, 2.9 μM, 3 μM, 3.1 μM, 3.2 μM, 3.3 μM, 3.4 μM, 3.5 μM, 3.6 μM, 3.7 μM, 3.8 μM, 3.9 μM, 4 μM, 4.1 μM, 4.2 μM, 4.3 μM, 4.4 μM, 4.5 μM, 4.6 μM, 4.7 μM, 4.8 μM, 4.9 μM, 5 μM, 5.1 μM, 5.2 μM, 5.3 μM, 5.4 μM, 5.5 μM, 5.6 μM, 5.7 μM, 5.8 μM, 5.9 μM, 6 μM, 6.1 μM, 6.2 μM, 6.3 μM, 6.4 μM, 6.5 μM, 6.6 μM, 6.7 μM, 6.8 μM, 6.9 μM, 7 μM, 7.1 μM, 7.2 μM, 7.3 μM, 7.4 μM, 7.5 μM, 7.6 μM, 7.7 μM, 7.8 μM, 7.9 μM, 8 μM, 8.1 μM, 8.2 μM, 8.3 μM, 8.4 μM, 8.5 μM, 8.6 μM, 8.7 μM, 8.8 μM, 8.9 μM, 9 μM, 9.1 μM, 9.2 μM, 9.3 μM, 9.4 μM, 9.5 μM, 9.6 μM, 9.7 μM, 9.8 μM, 9.9 μM, or 10 μM).

### Transduction Using an Activator of Prostaglandin E Receptor Signaling

In some embodiments, therapeutic cells of the disclosure are produced by transducing the cells in the presence of an activator of prostaglandin E receptor signaling.

In some embodiments, the activator of prostaglandin E receptor signaling is a small molecule, such as a compound described in WO 2007/112084 or WO 2010/108028, the disclosures of each of which are incorporated herein by reference as they pertain to prostaglandin E receptor signaling activators.

In some embodiments, the activator of prostaglandin E receptor signaling is a small molecule, such as a small organic molecule, a prostaglandin, a Wnt pathway agonist, a cAMP/PI3K/AKT pathway agonist, a Ca²⁺ second messenger pathway agonist, a nitric oxide (NO)/angiotensin signaling agonist, or another compound known to stimulate the prostaglandin signaling pathway, such as a compound selected from Mebeverine, Flurandrenolide, Atenolol, Pindolol, Gaboxadol, Kynurenic Acid, Hydralazine, Thiabendazole, Bicuclline, Vesamicol, Peruvoside, Imipramine, Chlorpropamide, 1,5-Pentamethylenetetrazole, 4-Aminopyridine, Diazoxide, Benfotiamine, 12-Methoxydodecenoic acid, N-Formyl-Met-Leu-Phe, Gallamine, IAA 94, Chlorotrianisene, and or a derivative of any of these compounds.

In some embodiments, the activator of prostaglandin E receptor signaling is a naturally-occurring or synthetic chemical molecule or polypeptide that binds to and/or interacts with a prostaglandin E receptor, typically to activate or increase one or more of the downstream signaling pathways associated with a prostaglandin E receptor.

In some embodiments, the activator of prostaglandin E receptor signaling is selected from the group consisting of prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling is PGE2 or dmPGE2.

In some embodiments, the activator of prostaglandin E receptor signaling is 15d-PGJ2, delta12-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(−)-[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, II-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxy PGE2.

In some embodiments, the activator of prostaglandin E receptor signaling is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling is a non-PGE2-based ligand. In some embodiments, the activator of prostaglandin E receptor signaling is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP3 agonist include, but are not limited to, AE5-599, MB28767, GR 63799X, ONO-NT012, and ONO-AE-248. Illustrative examples of non-PGE2-based EP4 agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists

In some embodiments, the activator of prostaglandin E receptor signaling is a Wnt agonist. Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt1Oa, Wnt1Ob, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃, or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, Calif.)). The structure of CHIR-99021 is

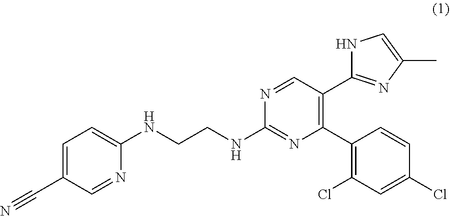 [SMILES: Cc1c[nH]c(-c2cnc(NCCNc3ccc(C#N)cn3)nc2-c2ccc(Cl)cc2Cl)n1]

or a salt thereof.

The structure of CHIR-98023 is

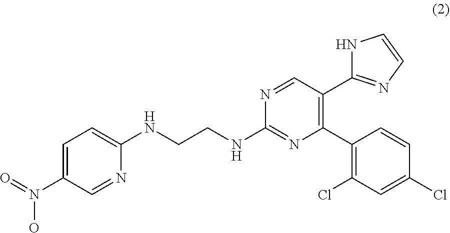

or a salt thereof.

In some embodiments, method further includes contacting the cell with a GSK3 inhibitor.

In some embodiments, the GSK3 inhibitor is CHIR-99021 or CHIR-98023.

In some embodiments, the GSK3 inhibitor is Li₂CO₃.

In some embodiments, the activator of prostaglandin E receptor signaling is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling is an agent that increases signaling through the NO/Angiotensin signaling, such as an agent selected from the group consisting of L-Arg, Sodium Nitroprusside, Sodium Vanadate, Bradykinin, and derivatives thereof.

### Transduction Using a Polycationic Polymer

In some embodiments, therapeutic cells of the disclosure are produced by transducing the cells in the presence of a polycationic polymer. In some embodiments, the polycationic polymer is polybrene, protamine sulfate, polyethylenimine, or a polyethylene glycol/poly-L-lysine block copolymer.

In some embodiments, the polycationic polymer is protamine sulfate.

In some embodiments, the cell is further contacted with an expansion agent during the transduction procedure. The cell may be, for example, a hematopoietic stem cell and the expansion agent may be a hematopoietic stem cell expansion agent, such as a hematopoietic stem cell expansion agent known in the art or described herein.

### Additional Transduction Enhancers

In some embodiments of the methods described herein, during the transduction procedure, the cell is further contacted with an agent that inhibits mTOR signaling. The agent that inhibits mTOR signaling may be, for example, rapamycin, among other suppressors of mTOR signaling.

Additional transduction enhancers that may be used in conjunction with the compositions and methods of the disclosure include, for example, tacrolimus and vectorfusin.

### Spinoculation

In some embodiments of the disclosure, a cell targeted for transduction may be spun e.g., by centrifugation, while being cultured with a viral vector (e.g., in combination with one or more additional agents described herein). This “spinoculation” process may occur with a centripetal force of, e.g., from about 200×g to about 2,000×g. The centripetal force may be, e.g., from about 300×g to about 1,200×g (e.g., about 300×g, 400×g, 500×g, 600×g, 700×g, 800×g, 900×g, 1,000×g, 1,100×g, or 1,200×g, or more). In some embodiments, the cell is spun for from about 10 minutes to about 3 hours (e.g., about 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, or more). In some embodiments, the cell is spun at room temperature, such as at a temperature of about 25° C.

Exemplary transduction protocols involving a spinoculation step are described, e.g., in Millington et al., PLoS One 4:e6461 (2009); Guo et al., Journal of Virology 85:9824-9833 (2011); O'Doherty et al., Journal of Virology 74:10074-10080 (2000); and Federico et al., Lentiviral Vectors and Exosomes as Gene and Protein Delivery Tools, Methods in Molecular Biology 1448, Chapter 4 (2016), the disclosures of each of which are incorporated herein by reference.

### Viral Vectors for NOD2 Expression

Viral genomes provide a rich source of vectors that can be used for the efficient delivery of exogenous genes into a mammalian cell. Viral genomes are particularly useful vectors for gene delivery as the polynucleotides contained within such genomes are typically incorporated into the nuclear genome of a mammalian cell by generalized or specialized transduction. These processes occur as part of the natural viral replication cycle, and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus, coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses are: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology, Third Edition (Lippincott-Raven, Philadelphia, (1996))). Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., (U.S. Pat. No. 5,801,030), the teachings of which are incorporated herein by reference.

### Retroviral Vectors

The delivery vector used in the methods and compositions described herein may be a retroviral vector. One type of retroviral vector that may be used in the methods and compositions described herein is a lentiviral vector. Lentiviral vectors (LVs), a subset of retroviruses, transduce a wide range of dividing and non-dividing cell types with high efficiency, conferring stable, long-term expression of the transgene. An overview of optimization strategies for packaging and transducing LVs is provided in Delenda, The Journal of Gene Medicine 6: S125 (2004), the disclosure of which is incorporated herein by reference.

The use of lentivirus-based gene transfer techniques relies on the in vitro production of recombinant lentiviral particles carrying a highly deleted viral genome in which the transgene of interest is accommodated. In particular, the recombinant lentivirus are recovered through the in trans coexpression in a permissive cell line of (1) the packaging constructs, i.e., a vector expressing the Gag-Pol precursors together with Rev (alternatively expressed in trans); (2) a vector expressing an envelope receptor, generally of an heterologous nature; and (3) the transfer vector, consisting in the viral cDNA deprived of all open reading frames, but maintaining the sequences required for replication, encapsidation, and expression, in which the sequences to be expressed are inserted.

A LV used in the methods and compositions described herein may include one or more of a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). The lentiviral vector optionally includes a central polypurine tract (cPPT) and a woodchuck hepatitis virus post-transcriptional regulatory element (WPRE), as described in U.S. Pat. No. 6,136,597, the disclosure of which is incorporated herein by reference as it pertains to WPRE. The lentiviral vector may further include a pHR′ backbone, which may include for example as provided below.

The Lentigen LV described in Lu et al., Journal of Gene Medicine 6:963 (2004) may be used to express the DNA molecules and/or transduce cells. A LV used in the methods and compositions described herein may a 5′-Long terminal repeat (LTR), HIV signal sequence, HIV Psi signal 5′-splice site (SD), delta-GAG element, Rev Responsive Element (RRE), 3′-splice site (SA), elongation factor (EF) 1-alpha promoter and 3′-self inactivating LTR (SIN-LTR). It will be readily apparent to one skilled in the art that optionally one or more of these regions is substituted with another region performing a similar function.

Enhancer elements can be used to increase expression of modified DNA molecules or increase the lentiviral integration efficiency. The LV used in the methods and compositions described herein may include a nef sequence. The LV used in the methods and compositions described herein may include a cPPT sequence which enhances vector integration. The cPPT acts as a second origin of the (+)-strand DNA synthesis and introduces a partial strand overlap in the middle of its native HIV genome. The introduction of the cPPT sequence in the transfer vector backbone strongly increased the nuclear transport and the total amount of genome integrated into the DNA of target cells. The LV used in the methods and compositions described herein may include a Woodchuck Posttranscriptional Regulatory Element (WPRE). The WPRE acts at the transcriptional level, by promoting nuclear export of transcripts and/or by increasing the efficiency of polyadenylation of the nascent transcript, thus increasing the total amount of mRNA in the cells. The addition of the WPRE to LV results in a substantial improvement in the level of transgene expression from several different promoters, both in vitro and in vivo. The LV used in the methods and compositions described herein may include both a cPPT sequence and WPRE sequence. The vector may also include an IRES sequence that permits the expression of multiple polypeptides from a single promoter.

In addition to IRES sequences, other elements which permit expression of multiple polypeptides are useful. The vector used in the methods and compositions described herein may include multiple promoters that permit expression more than one polypeptide. The vector used in the methods and compositions described herein may include a protein cleavage site that allows expression of more than one polypeptide. Examples of protein cleavage sites that allow expression of more than one polypeptide are described in Klump et al., Gene Ther.; 8:811 (2001), Osborn et al., Molecular Therapy 12:569 (2005), Szymczak and Vignali, Expert Opin Biol Ther. 5:627 (2005), and Szymczak et al., Nat Biotechnol. 22:589 (2004), the disclosures of which are incorporated herein by reference as they pertain to protein cleavage sites that allow expression of more than one polypeptide. It will be readily apparent to one skilled in the art that other elements that permit expression of multiple polypeptides identified in the future are useful and may be utilized in the vectors suitable for use with the compositions and methods described herein.

The vector used in the methods and compositions described herein may, be a clinical grade vector.

### Methods of Producing Functional NOD2-Expressing Cells by Ex Vivo Transfection

One platform that can be used to achieve therapeutically effective intracellular concentrations of one or more proteins described herein in mammalian cells is via the stable expression of genes encoding these agents (e.g., by integration into the nuclear or mitochondrial genome of a mammalian cell). These genes are polynucleotides that encode the primary amino acid sequence of the corresponding protein. In order to introduce such exogenous genes into a mammalian cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

Genes encoding therapeutic proteins of the disclosure can also be introduced into mammalian cells by targeting a vector containing a gene encoding such an agent to cell membrane phospholipids. For example, vectors can be targeted to the phospholipids on the extracellular surface of the cell membrane by linking the vector molecule to a VSV-G protein, a viral protein with affinity for all cell membrane phospholipids. Such, a construct can be produced using methods well known to those of skill in the field.

Recognition and binding of the polynucleotide encoding one or more therapeutic proteins of the disclosure by mammalian RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a mammalian promoter, the sequence of which can be recognized and bound by specific transcription initiation factors and ultimately RNA polymerase. Examples of mammalian promoters have been described in Smith et al., Mol. Sys. Biol., 3:73, online publication, the disclosure of which is incorporated herein by reference.

Once a polynucleotide encoding one or more therapeutic proteins has been incorporated into the nuclear DNA of a mammalian cell, transcription of this polynucleotide can be induced by methods known in the art. For example expression can be induced by exposing the mammalian cell to an external chemical reagent, such as an agent that modulates the binding of a transcription factor and/or RNA polymerase to the mammalian promoter and thus regulates gene expression. The chemical reagent can serve to facilitate the binding of RNA polymerase and/or transcription factors to the mammalian promoter, e.g., by removing a repressor protein that has bound the promoter. Alternatively, the chemical reagent can serve to enhance the affinity of the mammalian promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, Calif.) and can be administered to a mammalian cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode one or more therapeutic proteins and additionally include a mammalian enhancer sequence. Many enhancer sequences are now known from mammalian genes, and examples are enhancers from the genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982).

### Cells for Expression and Delivery of NOD2

Cells that may be used in conjunction with the compositions and methods described herein include cells that are capable of undergoing further differentiation. For example, one type of cell that can be used in conjunction with the compositions and methods described herein is a pluripotent cell. A pluripotent cell is a cell that possesses the ability to develop into more than one differentiated cell type. Examples of pluripotent cells are ESCs, iPSCs, and CD34+ cells. ESCs and iPSCs have the ability to differentiate into cells of the ectoderm, which gives rise to the skin and nervous system, endoderm, which forms the gastrointestinal and respiratory tracts, endocrine glands, liver, and pancreas, and mesoderm, which forms bone, cartilage, muscles, connective tissue, and most of the circulatory system.

Cells that may be used in conjunction with the compositions and methods described herein include hematopoietic stem cells and hematopoietic progenitor cells. Hematopoietic stem cells (HSCs) are immature blood cells that have the capacity to self-renew and to differentiate into mature blood cells including diverse lineages including but not limited to granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), dendritic cells, microglia, osteoclasts, and lymphocytes (e.g., NK cells, B-cells and T-cells). Human HSCs are CD34+. In addition, HSCs also refer to long term repopulating HSC (LT-HSC) and short-term repopulating HSC (ST-HSC). Any of these HSCs can be used in conjunction with the compositions and methods described herein.

HSCs and other pluripotent progenitors can be obtained from blood products. A blood product is a product obtained from the body or an organ of the body containing cells of hematopoietic origin. Such sources include unfractionated bone marrow, umbilical cord, placenta, peripheral blood, or mobilized peripheral blood. All of the aforementioned crude or unfractionated blood products can be enriched for cells having HSC or myeloid progenitor cell characteristics in a number of ways. For example, the more mature, differentiated cells can be selected against based on cell surface molecules they express. The blood product may be fractionated by positively selecting for CD34+ cells, which include a subpopulation of hematopoietic stem cells capable of self-renewal, multi-potency, and that can be re-introduced into a transplant recipient whereupon they home to the hematopoietic stem cell niche and reestablish productive and sustained hematopoiesis. Such selection is accomplished using, for example, commercially available magnetic anti-CD34 beads (Dynal, Lake Success, N.Y.). Myeloid progenitor cells can also be isolated based on the markers they express. Unfractionated blood products can be obtained directly from a donor or retrieved from cryopreservative storage. HSCs and myeloid progenitor cells can also be obtained from by differentiation of ES cells, iPS cells or other reprogrammed mature cells types.

Cells that may be used in conjunction with the compositions and methods described herein include allogeneic cells and autologous cells. When allogeneic cells are used, the cells may optionally be HLA-matched to the subject receiving a cell treatment.

Cells that may be used in conjunction with the compositions and methods described herein include CD34+/CD90+ cells and CD34+/CD164+ cells. These cells may contain a higher percentage of HSCs. These cells are described in Radtke et al. Sci. Transl. Med. 9: 1-10, 2017, and Pellin et al. Nat. Comm. 1-: 2395, 2019, the disclosures of each of which are hereby incorporated by reference in their entirety.

The cells described herein and above may be genetically modified so as to express NOD2 using, for example, a variety of methodologies (see, for example, the sections entitled “Methods of Producing Functional NOD2-Expressing Cells by Viral Transduction,” “Methods of Producing Functional NOD2-Expressing Cells by Ex Vivo Transfection,” and “Promoting Functional NOD2 Expression Using Gene Editing Techniques”). Once the cells have been adapted to express physiological levels of functional NOD2, these cells have therapeutic utility, and are referred to herein as “therapeutic cells of the disclosure.”

### Promoting Functional NOD2 Expression Using Gene Editing Techniques

Another useful tool for the disruption and/or integration of target genes into the genome of a cell (e.g., a pluripotent stem cell) is the clustered regularly interspaced short palindromic repeats (CRISPR)/Cas system, a system that originally evolved as an adaptive defense mechanism in bacteria and archaea against viral infection. The CRISPR/Cas system includes palindromic repeat sequences within plasmid DNA and a CRISPR-associated protein (Cas; e.g., Cas9 or Cas12a). This ensemble of DNA and protein directs site specific DNA cleavage of a target sequence by first incorporating foreign DNA into CRISPR loci. Polynucleotides containing these foreign sequences and the repeat-spacer elements of the CRISPR locus are in turn transcribed in a host cell to create a guide RNA, which can subsequently anneal to a target sequence and localize the Cas nuclease to this site. In this manner, highly site-specific Cas-mediated DNA cleavage can be engendered in a foreign polynucleotide because the interaction that brings Cas within close proximity of the target DNA molecule is governed by RNA:DNA hybridization. As a result, one can design a CRISPR/Cas system to cleave any target DNA molecule of interest. This technique has been exploited in order to edit eukaryotic genomes (Hwang et al. Nature Biotechnology 31:227 (2013), the disclosure of which is incorporated herein by reference) and can be used as an efficient means of site-specifically editing pluripotent stem cell genomes in order to cleave DNA prior to the incorporation of a gene encoding a target gene. The use of CRISPR/Cas to modulate gene expression has been described in, e.g., WO 2017/182881 and U.S. Pat. No. 8,697,359, the disclosures of each of which are incorporated herein by reference.

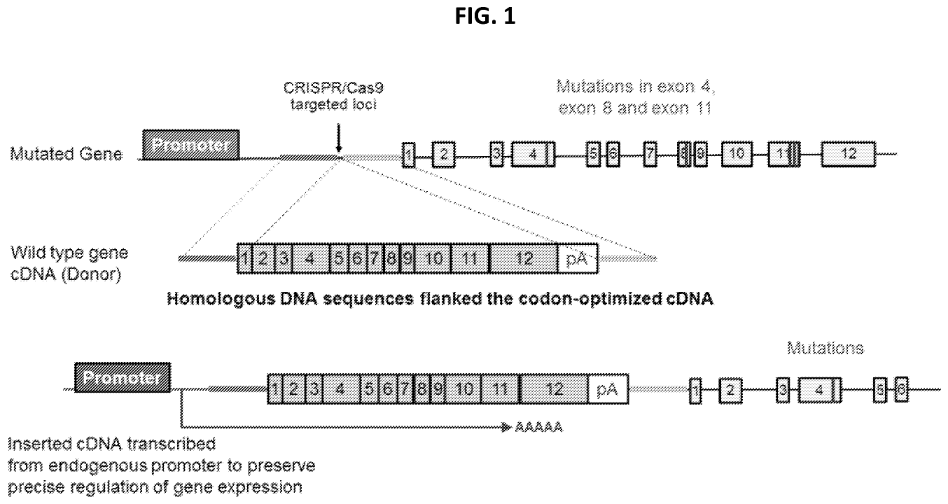

For example, using the compositions and methods of the disclosure, a genetic locus containing a nucleic acid that encodes a defective NOD2 protein may be edited so as to recapitulate functional NOD2 expression. An exemplary procedure for doing so is shown in FIG. 1. As depicted in FIG. 1, a genetic locus in a target cell, such as an autologous cell obtained from a patient suffering from Crohn's disease, may be edited at a site near or within the gene encoding endogenous NOD2. The gene encoding endogenous NOD2 may be one, for example, that has a mutation causing a NOD2 defect. To edit the target cell genome at this site, the cell may be provided a nuclease, such as a CRISPR-associated protein described above, along with a guide RNA (gRNA) and a template nucleic acid that encodes functional NOD2. The gRNA may direct the nuclease to the desired site within the target cell genome that is within or near a gene encoding a defective NOD2 protein. This may be achieved, for example, by base pair hybridization between the gRNA and the desired site in the target cell genome. Upon hybridization between the gRNA and the desired site, the nuclease may then catalyze a single-strand break or double-strand break at the desired site. Following this cleavage event, the template nucleic acid encoding functional NOD2 may then insert into the target cell genome at the desired site. In some embodiments, such as in the scenario depicted in FIG. 1, the template nucleic acid encoding functional NOD2 is inserted at a site that is operably joined to the endogenous NOD2 promoter, resulting in recapitulation of functional NOD2 protein expression.

Alternatively, base editing may be used to site-specifically edit one or more nucleobase at a desired site in the target cell genome so as to negate a NOD2 defect-causing mutation and recapitulate expression of a gene encoding functional NOD2. Base editing techniques may use, for example, a mutant Cas9 that induces a single-strand break in one strand of endogenous DNA in the target cell, at which point a fused deaminase then converts one base to another, such as adenine (A) to inosine (I), a proxy for guanine (G) following DNA replication. The accompanying T to C change in the remaining DNA strand occurs by way of DNA repair and replication. Base editing may also be used at the level of RNA, as mutant Cas13-ADAR fusion proteins have been deployed to bind RNA and catalyzing nucleobase modifications resulting in a change of A to I. Exemplary methods for DNA base editing that may be used to negate a defect-causing NOD2 mutation in the cells and recapitulate expression of a functional NOD2 protein are described in Cohen, “Novel CRISPR-derived ‘base editors’ surgically alter DNA or RNA, offering new ways to fix mutations,’ Science Magazine, October 2017, the disclosure of which is incorporated herein by reference.

Alternative methods for disruption of a target DNA by site-specifically cleaving genomic DNA prior to the incorporation of a gene of interest in a pluripotent stem cell include the use of zinc finger nucleases (ZFNs) and transcription activator-like effector nucleases (TALENs). Unlike the CRISPR/Cas system, these enzymes do not contain a guiding polynucleotide to localize to a specific target sequence. Target specificity is instead controlled by DNA binding domains within these enzymes. The use of ZFNs and TALENs in genome editing applications is described, e.g., in Urnov et al. Nature Reviews Genetics 11:636 (201 O); and in Joung et al. Nature Reviews Molecular Cell Biology 14:49 (2013), the disclosures of each of which are incorporated herein by reference. In some embodiments, an endogenous gene is disrupted, e.g., in a pluripotent stem cell, using the gene editing techniques described above.

In some embodiments, a gene editing approach, such as a CRISPR/Cas system or another of the nucleases described above, is used in order to insert a gene encoding a functional NOD2 protein (i.e., a NOD2 protein lacking an activity-disrupting mutation) directly into an endogenous NOD2 locus in a cell obtained from a patient suffering from Crohn's disease. In this way, expression of mutant NOD2 may be suppressed while simultaneously inducing expression of a functional NOD2 protein.

Agents that Enhance Cellular Engraftment

In some embodiments, the one or more agents administered to a patient that increase activity or expression of functional NOD2 is a population of cells (e.g., CD34+ cells) that express a NOD2 transgene. In such instances, prior to administration of the cells to the patient, the patient may be administered an agent that ablates an endogenous population of CD34+ cells, allowing the administered CD34+ cells to engraft in the patient. Examples of conditioning agents include myeloablative conditioning agents, which deplete a wide variety of hematopoietic cells in a patient. For instance, that patient may be pre-treated with an alkylating agent, such as a nitrogen mustard (e.g., bendamustine, chlorambucil, cyclophosphamide, ifosfamide, mechlorethamine, or melphalan), a nitrosourea (e.g., carmustine, lomustine, or streptozocin), an alkyl sulfonate (e.g., busulfan), a triazine (e.g., dacarbazine or temozolomide), or an ethylenimine (e.g., altretamine or thiotepa). In some embodiments, the patient is administered a conditioning agent that selectively ablates a specific population of endogenous cells, such as a population of endogenous CD34+ HSCs or HPCs.

In some embodiments, the patient is pre-treated with an activator of prostaglandin E receptor signaling in order to help facilitate the engraftment of administered NOD2-expressing cells. The prostaglandin E receptor signaling activator may be, for example, selected from the group consisting of prostaglandin (PG) A2 (PGA2), PGB2, PGD2, PGE1 (Alprostadil), PGE2, PGF2, PGI2 (Epoprostenol), PGH2, PGJ2, and derivatives and analogs thereof.

In some embodiments, the activator of prostaglandin E receptor signaling used to help facilitate engraftment of a NOD2-expressing cell is PGE2 or dmPG2.

In some embodiments, the activator of prostaglandin E receptor signaling used to help facilitate engraftment of a NOD2-expressing cell is 15d-PGJ2, delta12-PGJ2, 2-hydroxyheptadecatrienoic acid (HHT), Thromboxane (TXA2 and TXB2), PGI2 analogs, e.g., Iloprost and Treprostinil, PGF2 analogs, e.g., Travoprost, Carboprost tromethamine, Tafluprost, Latanoprost, Bimatoprost, Unoprostone isopropyl, Cloprostenol, Oestrophan, and Superphan, PGE1 analogs, e.g., 11-deoxy PGE1, Misoprostol, and Butaprost, and Corey alcohol-A ([3aa,4a,5,6aa]-(+[Hexahydro-4-(hydroxymetyl)-2-oxo-2H-cyclopenta/b/furan-5-yl][1,1′-biphenyl]-4-carboxylate), Corey alcohol-B (2H-Cyclopenta[b]furan-2-on,5-(benzoyloxy)hexahydro-4-(hydroxymethyl)[3aR-(3aa,4a,5,6aa)]), and Corey diol ((3aR,4S,5R,6aS)-hexahydro-5-hydroxy-4-(hydroxymethyl)-2H-cyclopenta[b]furan-2-one).

In some embodiments, the activator of prostaglandin E receptor signaling used to help facilitate engraftment of a NOD2-expressing cell is a prostaglandin E receptor ligand, such as prostaglandin E2 (PGE2), or an analogs or derivative thereof. Prostaglandins refer generally to hormone-like molecules that are derived from fatty acids containing 20 carbon atoms, including a 5-carbon ring, as described herein and known in the art. Illustrative examples of PGE2 “analogs” or “derivatives” include, but are not limited to, 16,16-dimethyl PGE2, 16-16 dimethyl PGE2 p-(p-acetamidobenzamido) phenyl ester, I I-deoxy-16,16-dimethyl PGE2, 9-deoxy-9-methylene-16, 16-dimethyl PGE2, 9-deoxy-9-methylene PGE2, 9-keto Fluprostenol, 5-trans PGE2, 17-phenyl-omega-trinor PGE2, PGE2 serinol amide, PGE2 methyl ester, 16-phenyl tetranor PGE2, 15(S)-15-methyl PGE2, 15 (R)-15-methyl PGE2, 8-iso-15-keto PGE2, 8-iso PGE2 isopropyl ester, 20-hydroxy PGE2, nocloprost, sulprostone, butaprost, 15-keto PGE2, and 19 (R) hydroxy PGE2.

In some embodiments, the activator of prostaglandin E receptor signaling used to help facilitate engraftment of a NOD2-expressing cell is a prostaglandin analog or derivative having a similar structure to PGE2 that is substituted with halogen at the 9-position (see, e.g., WO 2001/12596, herein incorporated by reference in its entirety), as well as 2-decarboxy-2-phosphinico prostaglandin derivatives, such as those described in US 2006/0247214, herein incorporated by reference in its entirety).

In some embodiments, the activator of prostaglandin E receptor signaling used to help facilitate engraftment of a NOD2-expressing cell is a non-PGE2-based ligand. In some embodiments, the activator of prostaglandin E receptor signaling used to help facilitate engraftment of a NOD2-expressing cell is CAY10399, ONO_8815Ly, ONO-AE1-259, or CP-533,536. Additional examples of non-PGE2-based EP2 agonists include the carbazoles and fluorenes disclosed in WO 2007/071456, herein incorporated by reference for its disclosure of such agents. Illustrative examples of non-PGE2-based EP₃ agonist include, but are not limited to, AE5-599, MB28767, GR 63799X, ONO-NT012, and ONO-AE-248. Illustrative examples of non-PGE₂-based EP₄ agonist include, but are not limited to, ONO-4819, APS-999 Na, AH23848, and ONO-AE 1-329. Additional examples of non-PGE2-based EP4 agonists can be found in WO 2000/038663; U.S. Pat. Nos. 6,747,037; and 6,610,719, each of which are incorporated by reference for their disclosure of such agonists

In some embodiments, the activator of prostaglandin E receptor signaling used to help facilitate engraftment of a NOD2-expressing cell is a Wnt agonist. Illustrative examples of Wnt agonists include, but are not limited to, Wnt polypeptides and glycogen synthase kinase 3 (GSK3) inhibitors. Illustrative examples of Wnt polypeptides suitable for use as compounds that stimulate the prostaglandin EP receptor signaling pathway include, but are not limited to, Wnt1, Wnt2, Wnt2b/13, Wnt3, Wnt3a, Wnt4, Wnt5a, Wnt5b, Wnt6, Wnt7a, Wnt7b, Wnt7c, Wnt8, Wnt8a, Wnt8b, Wnt8c, Wnt1Oa, Wnt1Ob, Wnt11, Wnt14, Wnt15, or biologically active fragments thereof. GSK3 inhibitors suitable for use as agents that stimulate the prostaglandin EP receptor signaling pathway bind to and decrease the activity of GSK3a, or GSK3. Illustrative examples of GSK3 inhibitors include, but are not limited to, BIO (6-bromoindirubin-3′-oxime), LiCl, Li₂CO₃ or other GSK-3 inhibitors, as exemplified in U.S. Pat. Nos. 6,057,117 and 6,608,063, as well as US 2004/0092535 and US 2004/0209878, and ATP-competitive, selective GSK-3 inhibitors CHIR-911 and CHIR-837 (also referred to as CT-99021/CHIR-99021 and CT-98023/CHIR-98023, respectively) (Chiron Corporation (Emeryville, Calif.)).

In some embodiments, the activator of prostaglandin E receptor signaling used to help facilitate engraftment of a NOD2-expressing cell is an agent that increases signaling through the cAMP/P13K/AKT second messenger pathway, such as an agent selected from the group consisting of dibutyryl cAMP (DBcAMP), phorbol ester, forskolin, sclareline, 8-bromo-cAMP, cholera toxin (CTx), aminophylline, 2,4 dinitrophenol (DNP), norepinephrine, epinephrine, isoproterenol, isobutylmethylxanthine (IBMX), caffeine, theophylline (dimethylxanthine), dopamine, rolipram, iloprost, pituitary adenylate cyclase activating polypeptide (PACAP), and vasoactive intestinal polypeptide (VIP), and derivatives of these agents.

In some embodiments, the activator of prostaglandin E receptor signaling used to help facilitate engraftment of a NOD2-expressing cell is an agent that increases signaling through the Ca²⁺ second messenger pathway, such as an agent selected from the group consisting of Bapta-AM, Fendiline, Nicardipine, and derivatives of these agents.

In some embodiments, the activator of prostaglandin E receptor signaling used to help facilitate engraftment of a NOD2-expressing cell is an agent that increases signaling through the NO/Angiotensin signaling, such as an agent selected from the group consisting of L-Arg, Sodium Nitroprusside, Sodium Vanadate, Bradykinin, and derivatives thereof.

### Methods of Measuring NOD2 Gene Expression

Preferably, the compositions and methods of the disclosure are used to facilitate expression of functional NOD2 at physiologically normal levels in a patient (e.g., a human patient having Crohn's disease). The therapeutic agents of the disclosure, for example, may stimulate functional NOD2 expression in a human patient (e.g., a human patient suffering from Crohn's disease) that has a NOD2 deficiency. For example, the therapeutic agents of the disclosure may facilitate NOD2 expression in a Crohn's disease patient at a level of, for example, from about 20% to about 200% of the level of functional NOD2 expression observed in a human subject of comparable age and body mass index that does not have a NOD2 deficiency (e.g., about 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, or 200% of the level of functional NOD2 expression observed in a human subject of comparable age and body mass index that does not have a NOD2 deficiency).

The expression level of functional NOD2 expressed in a patient can be ascertained, for example, by evaluating the concentration or relative abundance of mRNA transcripts derived from transcription of a functional NOD2 transgene. Additionally or alternatively, gene expression can be determined by evaluating the concentration or relative abundance of functional NOD2 protein produced by transcription and translation of a NOD2 transgene. Protein concentrations can also be assessed using functional assays, such as MDP detection assays. The sections that follow describe exemplary techniques that can be used to measure the expression level of a NOD2 transgene upon delivery to a patient, such as a patient having Crohn's disease as described herein. Transgene expression can be evaluated by a number of methodologies known in the art, including, but not limited to, nucleic acid sequencing, microarray analysis, proteomics, in-situ hybridization (e.g., fluorescence in-situ hybridization (FISH)), amplification-based assays, in situ hybridization, fluorescence activated cell sorting (FACS), northern analysis and/or PCR analysis of mRNAs.

### Nucleic Acid Detection

Nucleic acid-based methods for determining NOD2 transgene expression detection that may be used in conjunction with the compositions and methods described herein include imaging-based techniques (e.g., Northern blotting or Southern blotting). Such techniques may be performed using cells obtained from a patient following administration of the NOD2 transgene. Northern blot analysis is a conventional technique well known in the art and is described, for example, in Molecular Cloning, a Laboratory Manual, second edition, 1989, Sambrook, Fritch, Maniatis, Cold Spring Harbor Press, 10 Skyline Drive, Plainview, N.Y. 11803-2500. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis).

Transgene detection techniques that may be used in conjunction with the compositions and methods described herein to evaluate NOD2 expression further include microarray sequencing experiments (e.g., Sanger sequencing and next-generation sequencing methods, also known as high-throughput sequencing or deep sequencing). Exemplary next generation sequencing technologies include, without limitation, Illumina sequencing, Ion Torrent sequencing, 454 sequencing, SOLiD sequencing, and nanopore sequencing platforms. Additional methods of sequencing known in the art can also be used. For instance, transgene expression at the mRNA level may be determined using RNA-Seq (e.g., as described in Mortazavi et al., Nat. Methods 5:621-628 (2008) the disclosure of which is incorporated herein by reference in their entirety). RNA-Seq is a robust technology for monitoring expression by direct sequencing the RNA molecules in a sample. Briefly, this methodology may involve fragmentation of RNA to an average length of 200 nucleotides, conversion to cDNA by random priming, and synthesis of double-stranded cDNA (e.g., using the Just cDNA DoubleStranded cDNA Synthesis Kit from Agilent Technology). Then, the cDNA is converted into a molecular library for sequencing by addition of sequence adapters for each library (e.g., from Illumina®/Solexa), and the resulting 50-100 nucleotide reads are mapped onto the genome.

Transgene expression levels may be determined using microarray-based platforms (e.g., single-nucleotide polymorphism arrays), as microarray technology offers high resolution. Details of various microarray methods can be found in the literature. See, for example, U.S. Pat. No. 6,232,068 and Pollack et al., Nat. Genet. 23:41-46 (1999), the disclosures of each of which are incorporated herein by reference in their entirety. Using nucleic acid microarrays, mRNA samples are reverse transcribed and labeled to generate cDNA. The probes can then hybridize to one or more complementary nucleic acids arrayed and immobilized on a solid support. The array can be configured, for example, such that the sequence and position of each member of the array is known. Hybridization of a labeled probe with a particular array member indicates that the sample from which the probe was derived expresses that gene. Expression level may be quantified according to the amount of signal detected from hybridized probe-sample complexes. A typical microarray experiment involves the following steps: 1) preparation of fluorescently labeled target from RNA isolated from the sample, 2) hybridization of the labeled target to the microarray, 3) washing, staining, and scanning of the array, 4) analysis of the scanned image and 5) generation of gene expression profiles. One example of a microarray processor is the Affymetrix GENECHIP® system, which is commercially available and comprises arrays fabricated by direct synthesis of oligonucleotides on a glass surface. Other systems may be used as known to one skilled in the art.

Amplification-based assays also can be used to measure the expression level of a transgene in a target cell following delivery to a patient. In such assays, the nucleic acid sequences of the gene act as a template in an amplification reaction (for example, PCR, such as qPCR). In a quantitative amplification, the amount of amplification product is proportional to the amount of template in the original sample. Comparison to appropriate controls provides a measure of the expression level of the gene, corresponding to the specific probe used, according to the principles described herein. Methods of real-time qPCR using TaqMan probes are well known in the art. Detailed protocols for real-time qPCR are provided, for example, in Gibson et al., Genome Res. 6:995-1001 (1996), and in Heid et al., Genome Res. 6:986-994 (1996), the disclosures of each of which are incorporated herein by reference in their entirety. Levels of gene expression as described herein can be determined by RT-PCR technology. Probes used for PCR may be labeled with a detectable marker, such as, for example, a radioisotope, fluorescent compound, bioluminescent compound, a chemiluminescent compound, metal chelator, or enzyme.

### Protein Detection

Transgene expression can additionally be determined by measuring the concentration or relative abundance of a corresponding protein product (e.g., NOD2) encoded by a gene of interest. Protein levels can be assessed using standard detection techniques known in the art. Protein expression assays suitable for use with the compositions and methods described herein include proteomics approaches, immunohistochemical and/or western blot analysis, immunoprecipitation, molecular binding assays, ELISA, enzyme-linked immunofiltration assay (ELIFA), mass spectrometry, mass spectrometric immunoassay, and biochemical enzymatic activity assays. In particular, proteomics methods can be used to generate large-scale protein expression datasets in multiplex. Proteomics methods may utilize mass spectrometry to detect and quantify polypeptides (e.g., proteins) and/or peptide microarrays utilizing capture reagents (e.g., antibodies) specific to a panel of target proteins to identify and measure expression levels of proteins expressed in a sample (e.g., a single cell sample or a multi-cell population).

Exemplary peptide microarrays have a substrate-bound plurality of polypeptides, the binding of an oligonucleotide, a peptide, or a protein to each of the plurality of bound polypeptides being separately detectable. Alternatively, the peptide microarray may include a plurality of binders, including, but not limited to, monoclonal antibodies, polyclonal antibodies, phage display binders, yeast two-hybrid binders, aptamers, which can specifically detect the binding of specific oligonucleotides, peptides, or proteins. Examples of peptide arrays may be found in U.S. Pat. Nos. 6,268,210, 5,766,960, and 5,143,854, the disclosures of each of which are incorporated herein by reference in their entirety.

Mass spectrometry (MS) may be used in conjunction with the methods described herein to identify and characterize transgene expression in a cell from a patient (e.g., a human patient) following delivery of the transgene. Any method of MS known in the art may be used to determine, detect, and/or measure a protein or peptide fragment of interest, e.g., LC-MS, ESI-MS, ESI-MS/MS, MALDI-TOF-MS, MALDI-TOF/TOF-MS, tandem MS, and the like. Mass spectrometers generally contain an ion source and optics, mass analyzer, and data processing electronics. Mass analyzers include scanning and ion-beam mass spectrometers, such as time-of-flight (TOF) and quadruple (Q), and trapping mass spectrometers, such as ion trap (IT), Orbitrap, and Fourier transform ion cyclotron resonance (FT-ICR), may be used in the methods described herein. Details of various MS methods can be found in the literature. See, for example, Yates et al., Annu. Rev. Biomed. Eng. 11:49-79, 2009, the disclosure of which is incorporated herein by reference in its entirety.

Prior to MS analysis, proteins in a sample obtained from the patient can be first digested into smaller peptides by chemical (e.g., via cyanogen bromide cleavage) or enzymatic (e.g., trypsin) digestion. Complex peptide samples also benefit from the use of front-end separation techniques, e.g., 2D-PAGE, HPLC, RPLC, and affinity chromatography. The digested, and optionally separated, sample is then ionized using an ion source to create charged molecules for further analysis. Ionization of the sample may be performed, e.g., by electrospray ionization (ESI), atmospheric pressure chemical ionization (APCI), photoionization, electron ionization, fast atom bombardment (FAB)/liquid secondary ionization (LSIMS), matrix assisted laser desorption/ionization (MALDI), field ionization, field desorption, thermospray/plasmaspray ionization, and particle beam ionization. Additional information relating to the choice of ionization method is known to those of skill in the art.

After ionization, digested peptides may then be fragmented to generate signature MS/MS spectra. Tandem MS, also known as MS/MS, may be particularly useful for analyzing complex mixtures. Tandem MS involves multiple steps of MS selection, with some form of ion fragmentation occurring in between the stages, which may be accomplished with individual mass spectrometer elements separated in space or using a single mass spectrometer with the MS steps separated in time. In spatially separated tandem MS, the elements are physically separated and distinct, with a physical connection between the elements to maintain high vacuum. In temporally separated tandem MS, separation is accomplished with ions trapped in the same place, with multiple separation steps taking place over time. Signature MS/MS spectra may then be compared against a peptide sequence database (e.g., SEQUEST). Post-translational modifications to peptides may also be determined, for example, by searching spectra against a database while allowing for specific peptide modifications.

### Routes of Administration

The compositions described herein may be administered to a patient (e.g., a human patient suffering from Crohn's disease) by one or more of a variety of routes, such as intravenously or by means of a bone marrow transplant. The most suitable route for administration in any given case may depend on the particular composition administered, the patient, pharmaceutical formulation methods, administration methods (e.g., administration time and administration route), the patients age, body weight, sex, severity of the diseases being treated, the patient's diet, and the patient's excretion rate. Multiple routes of administration may be used to treat a single patient at one time, or the patient may receive treatment via one route of administration first, and receive treatment via another route of administration during a second appointment, e.g., 1 week later, 2 weeks later, 1 month later, 6 months later, or 1 year later. Compositions may be administered to a subject once, or cells may be administered one or more times (e.g., 2-10 times) per week, month, or year.

### Selection of Donor Cells

In some embodiments, the patient undergoing treatment is the donor that provides cells (e.g., pluripotent cells, such as CD34+ hematopoietic stem or progenitor cells) that are subsequently modified to express one or more therapeutic proteins of the disclosure before being re-administered to the patient. In such cases, withdrawn cells (e.g., hematopoietic stem or progenitor cells) may be re-infused into the subject following, for example, incorporation of a transgene encoding functional NOD2, such that the cells may subsequently home to hematopoietic tissue and establish productive hematopoiesis, thereby populating or repopulating a line of cells that is defective or deficient in the patient. In cases in which the patient undergoing treatment also serves as the cell donor, the transplanted cells (e.g., hematopoietic stem or progenitor cells) are less likely to undergo graft rejection. This stems from the fact that the infused cells are derived from the patient and express the same HLA class I and class II antigens as expressed by the patient. Alternatively, the patient and the donor may be distinct. In some embodiments, the patient and the donor are related, and may, for example, be HLA-matched. As described herein, HLA-matched donor-recipient pairs have a decreased risk of graft rejection, as endogenous T cells and NK cells within the transplant recipient are less likely to recognize the incoming hematopoietic stem or progenitor cell graft as foreign, and are thus less likely to mount an immune response against the transplant. Exemplary HLA-matched donor-recipient pairs are donors and recipients that are genetically related, such as familial donor-recipient pairs (e.g., sibling donor-recipient pairs). In some embodiments, the patient and the donor are HLA-mismatched, which occurs when at least one HLA antigen, in particular with respect to HLA-A, HLA-B and HLA-DR, is mismatched between the donor and recipient. To reduce the likelihood of graft rejection, for example, one haplotype may be matched between the donor and recipient, and the other may be mismatched.

### Pharmaceutical Compositions and Dosing

In cases in which a patient is administered a population of cells that together express one or more therapeutic proteins of the disclosure, the number of cells administered may depend, for example, on the expression level of the desired protein(s), the patient, pharmaceutical formulation methods, administration methods (e.g., administration time and administration route), the patients age, body weight, sex, severity of the disease being treated, and whether or not the patient has been treated with agents to ablate endogenous pluripotent cells (e.g., endogenous CD34+ cells, hematopoietic stem or progenitor cells, or microglia, among others). The number of cells administered may be, for example, from 1×10⁶ cells/kg to 1×10¹² cells/kg, or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more). Cells may be administered in an undifferentiated state, or after partial or complete differentiation into microglia. The number of pluripotent cells may be administered in any suitable dosage form.

Cells may be admixed with one or more pharmaceutically acceptable carriers, diluents, and/or excipients. Exemplary carriers, diluents, and excipients that may be used in conjunction with the compositions and methods of the disclosure are described, e.g., in Remington: The Science and Practice of Pharmacy (2012, 22nd ed.) and in The United States Pharmacopeia: The National Formulary (2015, USP 38 NF 33).

### EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure.

### Example 1. Generation of a Pluripotent Stem Cell Expressing Functional NOD2 for the Treatment of Crohn's Disease

An exemplary method for making pluripotent cells (e.g., embryonic stem cells (ESCs), induced pluripotent stem cells (iPSCs), or CD34+ cells) that express functional NOD2 is by way of transduction. Retroviral vectors (e.g., a lentiviral vector, alpharetroviral vector, or gammaretroviral vector) containing, e.g., a suitable promoter, such as a promoter described herein, and a polynucleotide encoding functional NOD2 can be engineered using vector production techniques described herein or known in the art. After the retroviral vector is engineered, the retrovirus can be used to transduce pluripotent cells (e.g., ESCs, iPSCs, or CD34+ cells) to generate a population of pluripotent cells that express functional NOD2.

Additional exemplary methods for making pluripotent cells that express functional NOD2 are transfection techniques. Using molecular biology procedures described herein and known in the art, plasmid DNA containing a promoter and a polynucleotide encoding functional NOD2 can be produced. For example, a nucleic acid encoding functional NOD2 may be amplified from a human cell line using PCR-based techniques known in the art, or a nucleic acid encoding functional NOD2 may be synthesized, for example, using solid-phase polynucleotide synthesis procedures. The nucleic acid and promoter can then be ligated into a plasmid of interest, for example, using suitable restriction endonuclease-mediated cleavage and ligation protocols. After the plasmid DNA is engineered, the plasmid can be used to transfect the pluripotent cells (e.g., ESCs, iPSCs, or CD34+ cells) using, for example, electroporation or another transfection technique described herein to generate a population of pluripotent cells that express the encoded protein(s).

### Example 2. Functional NOD2 is an Important Contributor to the Proinflammatory Cytokine Response, and NOD2 Restoration Rescues the Ability of NOD2-Impaired Cells—Such as Those Observed in Crohn's Disease—to Mount a Cytokine Response

### Introduction

This example describes the results of a series of experiments that were conducted with the aim of assessing the effects of NOD2 activation in various cell lines, including model systems of Crohn's disease in which functional NOD2 is depleted. As is described herein, Crohn's disease is a debilitating disorder characterized by the inability of endogenous cells to release inflammatory cytokines, particularly in response to muramyl dipeptide (MDP). As the results of these experiments demonstrate, functional NOD2 delivery to a system—such as by way of cell-based gene therapy, viral transduction, or a gene editing approach that inserts a functional NOD2 gene at a desired genetic locus—has the beneficial effect of restoring the ability of cells to mount an inflammatory immune response. In light of these results, NOD2 gene delivery represents a robust approach for the treatment of Crohn's disease.

### Results

NOD2 Signaling in Healthy Cells Stimulates a Proinflammatory Cytokine Response

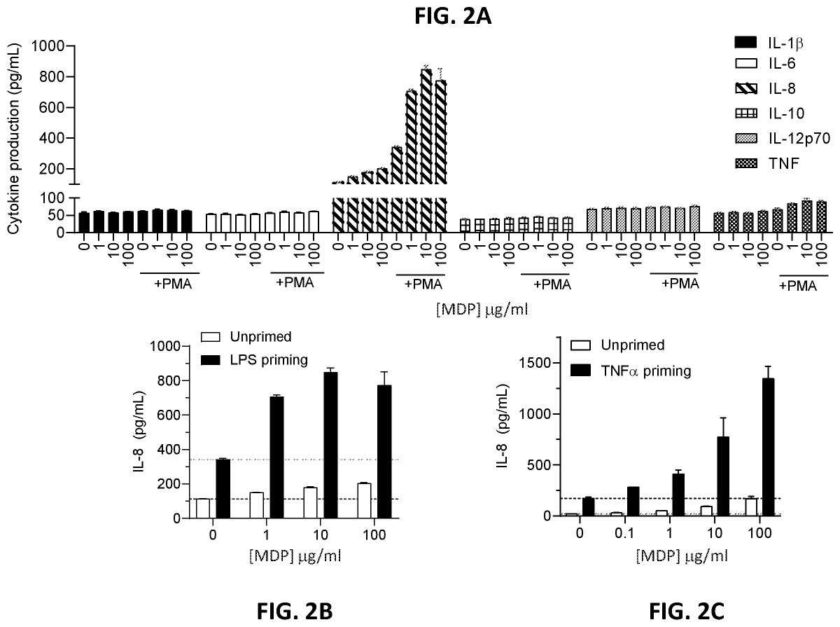

First, a set of experiments was conducted in order to demonstrate the ability of NOD2 activation to stimulate an inflammatory immune response in healthy human monocytes. FIGS. 2A-2C show that, indeed, NOD2 activation effectuates a robust inflammatory cytokine release in such cells. THP-1 human monocytic cells were pre-stimulated for 18 hours with 10 ng/mL phorbol 12-myristate (PMA), 10 ng/mL lipopolysaccharide (LPS) or 5 ng/mL TNFα followed by treatment with MDP to induce NOD2 signaling. NOD2-dependent cytokine production was assayed in cell supernatants after 18-24 hours by flow based cytometric bead array analysis and ELISA. As FIGS. 2A-2C demonstrate, NOD2 activation resulted in an augmented release of inflammatory cytokines in this system.

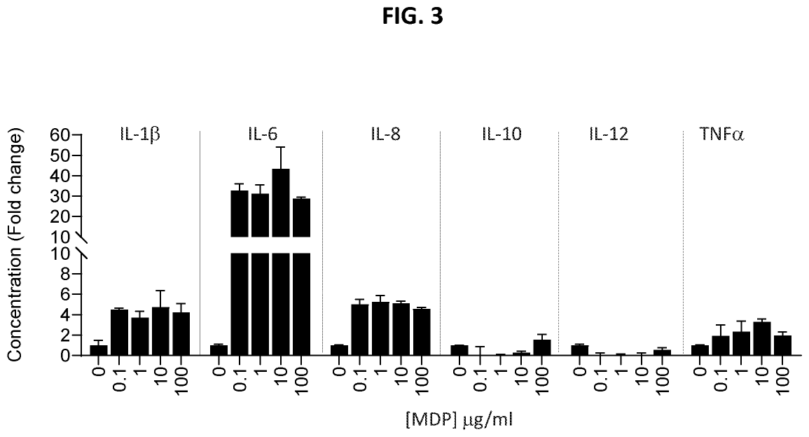

In addition, NOD2 activation was found to be capable of generating a robust inflammatory cytokine response even in the absence of priming. Peripheral blood CD14+ monocytes isolated from healthy human donors were treated with MDP to induce NOD2 signaling. NOD2-dependent cytokine production was then assayed in cell supernatants after 18-24 hours by flow based cytometric bead array analysis. As FIG. 3 shows, even without pre-stimulation, NOD2 activation resulted in a strong inflammatory cytokine response.

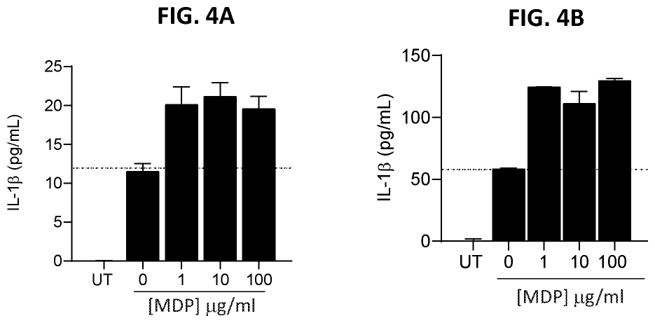

A robust proinflammatory response to NOD2 activation was also observed in wild-type murine tissue-isolated and bone marrow-derived monocytes (FIGS. 4A and 4B). Primary murine peritoneal macrophages (CD11b+) were primed overnight by LPS treatment, followed by stimulation of NOD2 signaling by MDP treatment (FIG. 4A). Separately, murine bone marrow-derived macrophages, generated by ex vivo culture in GM-CSF, were primed by overnight treatment with LPS (10 ng/mL), followed by stimulation of NOD2 signaling by MDP treatment. NOD2 stimulation resulted in release of active/processed IL-1β detected by ELISA.

Depletion of Functional NOD2 Impairs the Proinflammatory Cytokine Response

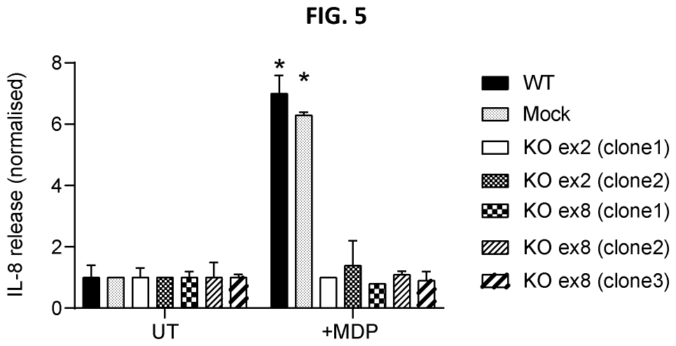

Having established that NOD2 activation engenders an inflammatory cytokine response, another set of experiments was then conducted to assess the effects of NOD2 disruption on inflammatory cytokine release. As FIG. 5 shows, NOD2 disruption impairs the THP-1 monocyte inflammatory cytokine response to MDP. Several NOD2-mutant THP-1 clonal cell lines were generated using CRISPR-Cas9 to model the NOD2-deficiency that is associated with Crohn's Disease. Wildtype (WT), several exon-2 and exon-8 targeted NOD2 knock out clones (KO), and THP-1 cells undergoing mock CRISPR-Cas9 NOD2 disruption (Mock) were primed with LPS overnight, followed by stimulation with MDP (10 μg/mL). NOD2 KO THP-1 clones showed an inability to generate a proinflammatory cytokine reaction to MDP stimulation, evidencing the role of NOD2 signaling in retaining the ability to mount an inflammatory cytokine response.

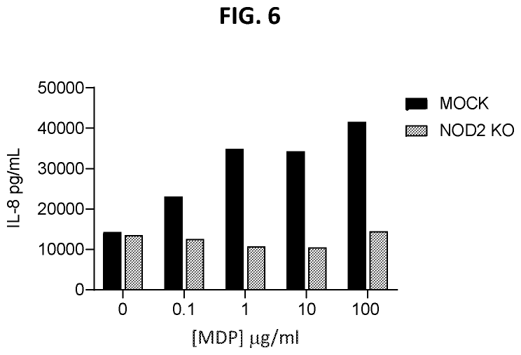

To demonstrate the effects of NOD2 disruption in CD34+ hematopoietic stem cells (HSCs), peripheral blood-derived CD34+ cells isolated from healthy human donors were subject to targeted disruption of NOD2 by CRISPR-Cas9 gene editing (RNP+guideRNA nucleofection). Gene edited cells, NOD2 KO cells, and MOCK edited cells (receiving RNP only) were then cultured for 14 days in the presence of cytokines to promote differentiation towards monocyte/macrophage lineage committed cells. Cell cultures were then stimulated with MDP (0-100 μg/mL) for 18-24 hours and cell supernatants were assayed for IL-8 cytokine release by ELISA. As FIG. 6 shows, disruption of NOD2 expression by way of CRISPR-Cas9 gene editing suppresses the release of inflammatory cytokines in response to MDP.

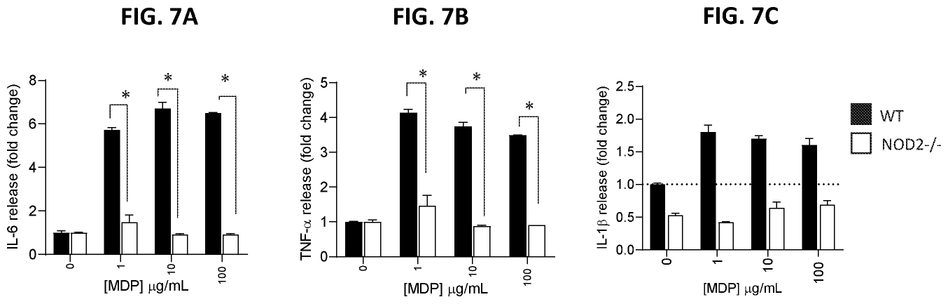

The effects of NOD2 disruption in murine cells was also investigated. As FIGS. 7A-7C show, NOD2−/− mice have an impaired macrophage inflammatory cytokine responses to MDP. WT and NOD2−/− murine bone marrow-derived macrophages and monocytes generated by ex vivo culture in GM-CSF or M-CSF, respectively, were primed by overnight treatment with LPS (1 ng/mL), followed by stimulation of NOD2 signaling by MDP treatment. NOD2 stimulation resulted in release of IL-6, TNFα, and active/processed IL-1β by WT-derived cells. Strikingly, this effect was absent in NOD2−/− cells, as detected by flow cytometric bead array analysis or ELISA.

Restoration of NOD2 Rescues the Ability of Cells to Release Proinflammatory Cytokines

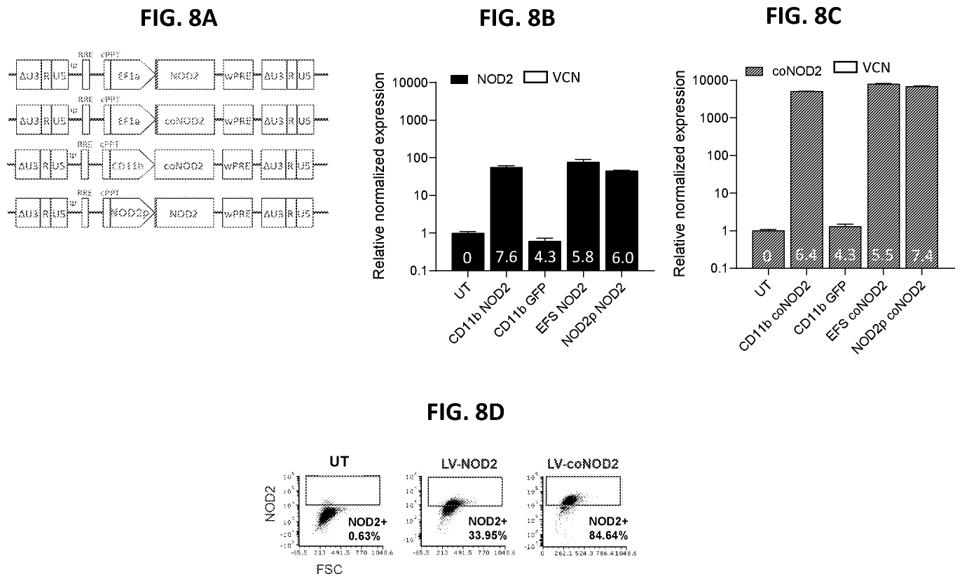

Having demonstrated that functional NOD2 is an important contributor to proinflammatory cytokine release, a series of experiments was then conducted to evaluate the ability of functional NOD2 delivery to restore the proinflammatory cytokine response in NOD2 deficient cells. FIGS. 8A-8D show the design and validation of a series of lentiviral vectors aimed at restoring functional NOD2 expression. As these figures illustrate, a series of different promoters were used to control NOD2 expression. Additionally, the NOD2 nucleic acid sequence may be codon-optimized to further enhance NOD2 expression. Examples of promoters that may be used include constitutive promoters (e.g., EF1α and EFS promoters) as well as myeloid lineage-specific promoters (e.g., CD11b promoter) and endogenous NOD2 (NOD2p) promoter. Briefly, THP-1 monocytes were transduced with lentiviral vectors (multiplicity of infection (moi) 10) and relative gene expression of WT NOD2 and codon-optimized (co) NOD2 were detected by transgene-specific RT-PCR analysis after 4 days (relative to untransduced cells). As FIGS. 8A-8D show, lentiviral transduction effectuated robust NOD2 expression.

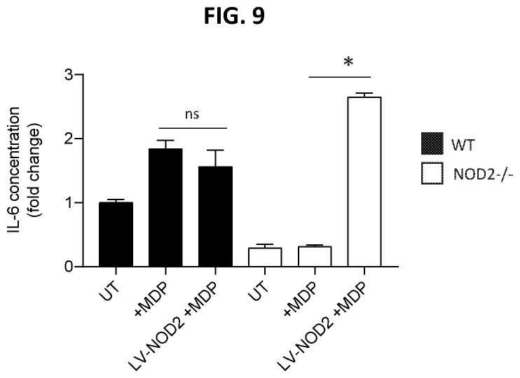

Not only was lentiviral transduction capable of restoring NOD2 expression, but this had the effect of improving inflammatory cytokine release. As FIG. 9 shows, lentiviral transduction of murine bone marrow HSCs restored functional NOD2 expression in NOD2−/− monocytes and rescued their ability to release IL-6. Briefly, bone marrow lineage negative HSC isolated from WT or NOD2−/− mice were transduced with a lentiviral vector encoding NOD2. Murine bone marrow-derived macrophages were then generated by ex vivo culture in GM-CSF. Cells were primed by overnight treatment with LPS (1 ng/mL), followed by stimulation of NOD2 signaling by MDP (10 μg/mL) treatment. NOD2-mediated IL-6 production was detected in cell supernatants by ELISA after 18-24 hours. FIG. 9 demonstrates that IL-6 production was enhanced as a result of the restored NOD2 expression.

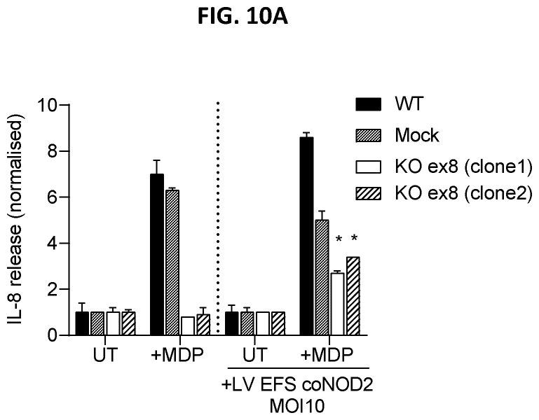

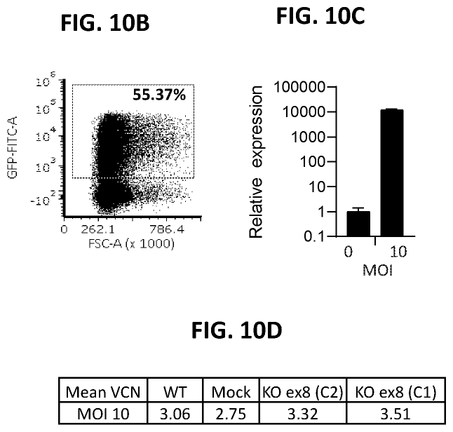

FIGS. 10A-10D demonstrate that this beneficial effect also applies to human monocytes. THP-1 WT and CRISPR-Cas9 gene edited clones (NOD2KO or mock edited) were transduced with LV-coNOD2 vector (moi 10). Three days after transduction, THP-1 cells were primed with LPS and then treated with MDP (10 μg/mL) to stimulate NOD2 activity. Lentiviral transduction of NOD2 KO THP-1 clones resulted in restoration of NOD2-dependent IL-8 cytokine release detected by ELISA (FIG. 10A). Lentiviral transduction efficiency of THP-1 cells was confirmed by assessing their transduction using a GFP reporter-LV construct. NOD2 gene expression was confirmed by RT-PCR analysis of transduced cells (relative to untransduced cells).

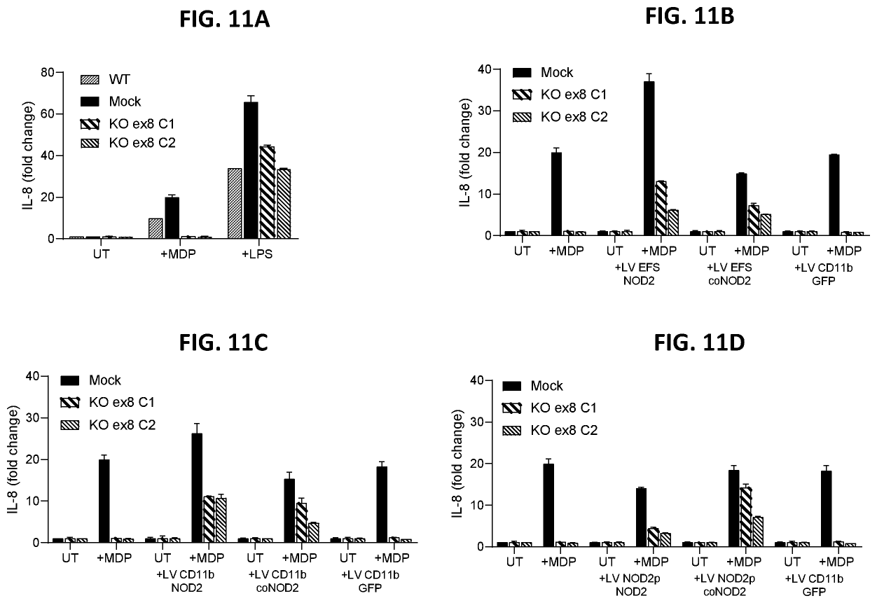

FIGS. 11A-11D reinforce this result and further demonstrate that lentiviral transduction of NOD2-deficient THP-1 cells can restore human monocyte inflammatory responses to MDP. THP-1 WT and CRISPR-Cas9 gene edited clones (NOD2KO or mock edited) were transduced with LV-coNOD2 vector (moi 10). Three days after transduction, THP-1 cells were primed with LPS and then treated with MDP (10 μg/mL) to stimulate NOD2 activity. Lentiviral transduction of NOD2 KO THP-1 clones resulted in restoration of NOD2-dependent IL-8 cytokine release detected by ELISA.

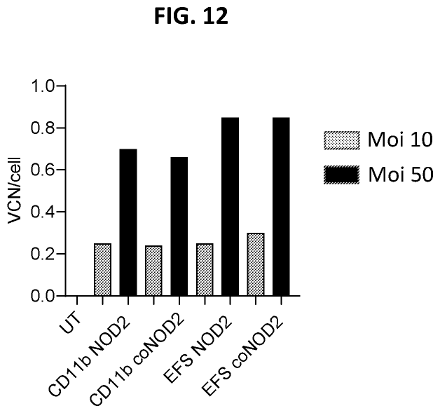

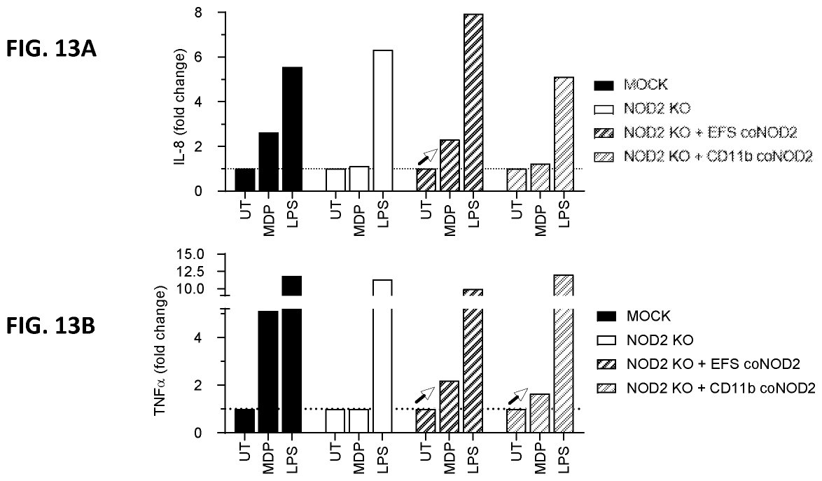

Significantly, the beneficial effects of functional NOD2 restoration are also observed in CD34+ HSCs. CD34+ cells isolated from mobilized peripheral blood of healthy human volunteers were transduced with lentiviral vectors (moi 10 & 50) generated to transfer WT or codon optimized NOD2 under the control of a myeloid lineage-specific promoter (CD11b promoter) or a constitutive promoter (EFS promoter) (FIG. 12). As FIGS. 13A and 13B show, the effect of NOD2 delivery to NOD2-knokout (NOD2-KO) peripheral blood-derived CD34+ cells is to restore MDP detection by differentiated CD34+ cell cultures. CD34+ cells isolated from mobilized peripheral blood of healthy human volunteers were firstly subject to gene editing by CRISPR-Cas9 to disrupt NOD2 (NOD2KO or mock) and then transduced with LV-coNOD2 vectors. CD34+ cells were then differentiated in vitro for 2 weeks (final cultures composed of 15-30% CD11b+CD14+ cells) after which cultures were treated with MDP to stimulate NOD2 activity. Lentiviral transduction of NOD2 KO cells resulted in partial restoration of NOD2-dependent (FIG. 13A) IL-8 and (FIG. 13B) TNFα cytokine release upon MDP stimulation (1 μg/mL) detected by ELISA. CD34+ cells were transduced with lentiviral vectors generated to transfer WT NOD2 or codon optimized NOD2 under the control of a myeloid lineage-specific promoter (CD11b promoter) or a constitutive promoter (EFS promoter).

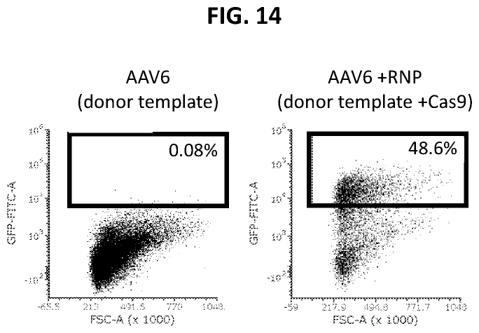

In addition to lentiviral delivery of NOD2, another approach for restoring NOD2 in a NOD2-deficient cell is by way of CRISPR-mediated gene editing, which can have the effect of inserting a functional NOD2 transgene at a desired genetic locus. FIG. 14 illustrates this proof of concept. As FIG. 14 shows, gene editing can be used to effectuate targeted GFP insertion into the NOD2 locus in CD34+ HSCs. A GFP reporter sequence was used to validate a gene editing strategy for targeted insertion of a payload donor template into exon2 of the NOD2 gene locus. Gene editing of peripheral blood derived CD34+ cells was performed using CRISPR-Cas9 RNP nucleofection. Donor payload delivery was achieved using a template sequence delivered by an AAV6 vector. Particularly, the donor payload included a transgene encoding GFP under the control of the EFS promoter. The efficiency of homology-directed repair was confirmed by flow cytometry detection of GFP+ cells in myeloid differentiated cell cultures. Data shown is representative of 2 independent experiments. Targeting to the NOD2 locus was confirmed by an ‘In-Out’ PCR approach, in which one primer is located in the targeted genomic locus outside the homology arm and the other primer is located inside the transgene cassette (data not shown).

By substituting a functional NOD2 transgene for the GFP reporter used in FIG. 14, one can deliver functional NOD2 to a desired genetic locus in a cell, thereby restoring NOD2 expression and rescuing the ability of the cell to mount a proinflammatory cytokine response to MDP.

### Conclusion

As the results of these experiments demonstrate, NOD2 is an important contributor to the ability of hematopoietic cells to mount a proinflammatory cytokine response, as the expression of NOD2 augments the release of proinflammatory cytokines and the disruption of NOD2 impairs the release of proinflammatory cytokines. Crohn's disease is associated with reduced NOD2 activity that, in turn, hinders the ability of cells to release proinflammatory cytokines. Importantly, the foregoing experiments demonstrate that the delivery of functional NOD2—whether by way of viral transduction, cell-based gene therapy, or a gene editing approach that inserts a functional NOD2 transgene at a desired genetic locus—rescues the ability of cells to mount a proinflammatory cytokine response.

### Example 3. Administration of a Therapeutic Composition to a Patient Suffering from Crohn's Disease

According to the methods disclosed herein, a patient, such as a human patient, can be treated so as to reduce or alleviate symptoms of Crohn's disease and/or so as to target an underlying biochemical etiology of the disease. To this end, the patient may be administered, for example, a population of pluripotent cells, (e.g., ESCs, iPSCs, CD34+ cells) expressing functional NOD2. The population of pluripotent cells may be administered to the patient, for example, systemically (e.g., intravenously). The cells may be administered in a therapeutically effective amount, such as from 1×10⁶ cells/kg to 1×10¹² cells/kg or more (e.g., 1×10⁷ cells/kg, 1×10⁸ cells/kg, 1×10⁹ cells/kg, 1×10¹⁰ cells/kg, 1×10¹¹ cells/kg, 1×10¹² cells/kg, or more).

Before the population of cells is administered to the patient, one or more agents may be administered to the patient to ablate the patient's endogenous hematopoietic cell population, for example, by administration of a conditioning agent described herein.

The success of the treatment may be monitored by way of various clinical indicators. Effective treatment of Crohn's disease using a composition of the disclosure may manifest, for example, as (i) sustained disease remission, such as sustained disease remission for at least one year; (ii) an observation that the patient no longer requires treatment with immunosuppressive agents, biologic agents, and/or corticosteroids; and/or (iii) an observation that the patient does not exhibit evidence of erosive disease in the gastrointestinal tract, as assessed by endoscopy and/or radiology.

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

What is claimed is:

1. A method of treating Crohn's disease in a patient in need thereof, the method comprising providing to the patient one or more agents that increase expression and/or activity of Nucleotide-binding oligomerization domain-containing protein 2 (NOD2).

2. A method of inducing sustained disease remission of Crohn's disease in a patient in need thereof, the method comprising providing to the patient one or more agents that increase expression and/or activity of NOD2.

3. A method of increasing muramyl dipeptide (MDP) sensing in a patient that has Crohn's disease, the method comprising providing to the patient one or more agents that increase expression and/or activity of NOD2.

4. A method of increasing NFκB signal transduction detection in a patient that has Crohn's disease, the method comprising providing to the patient one or more agents that increase expression and/or activity of NOD2.

5. The method of any one of claims 1-4, wherein the one or more agents comprise a nucleic acid molecule that encodes NOD2.

6. The method of claim 5, wherein the nucleic acid molecule is provided to the patient by administering to the patient a composition comprising a population of cells that express NOD2.

7. The method of claim 6, wherein the cells are pluripotent cells.

8. The method of claim 6 or 7, wherein the cells are human cells.

9. The method of any one of claims 6-8, wherein the cells are hematopoietic stem cells (HSCs) or hematopoietic progenitor cells (HPCs).

10. The method of any one of claims 6-8, wherein the cells are embryonic stem cells.

11. The method of any one of claims 6-8, wherein the cells are induced pluripotent stem cells.

12. The method of any one of claims 6-8, wherein the cells are CD34+ cells.

13. The method of claim 12, wherein the CD34+ cells are myeloid progenitor cells.

14. The method of any one of claims 6-13, wherein the composition is administered systemically to the patient.

15. The method of claim 14, wherein the composition is administered to the patient by way of intravenous injection.

16. The method of any one of claims 6-15, wherein the cells are autologous with respect to the patient.

17. The method of any one of claims 6-15, wherein the cells are allogeneic with respect to the patient.

18. The method of claim 17, wherein the cells are HLA-matched to the patient.

19. The method of any one of claims 6-18, wherein the cells are transduced ex vivo to express NOD2.

20. The method of claim 19, wherein the cells are transduced with a viral vector selected from the group consisting of a Retroviridae family virus, an adenovirus, a parvovirus, a coronavirus, a rhabdovirus, a paramyxovirus, a picornavirus, an alphavirus, a herpes virus, and a poxvirus.

21. The method of claim 20, wherein the viral vector is a Retroviridae family viral vector.

22. The method of claim 21, wherein the Retroviridae family viral vector is a lentiviral vector.

23. The method of claim 21, wherein the Retroviridae family viral vector is an alpharetroviral vector or a gammaretroviral vector.

24. The method of any one of claims 20-23, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

25. The method of any one of claims 20-24, wherein the viral vector is a pseudotyped viral vector.

26. The method of claim 25, wherein the pseudotyped viral vector selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

27. The method of claim 25 or 26, wherein the pseudotyped viral vector comprises one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

28. The method of claim 27, wherein the pseudotyped viral vector comprises a VSV-G envelope protein.

29. The method of any one of claims 6-18, wherein the cells are transfected ex vivo to express NOD2.

30. The method of claim 29, wherein the cells are transfected using a cationic polymer, diethylaminoethyldextran, polyethylenimine, a cationic lipid, a liposome, calcium phosphate, an activated dendrimer, and/or a magnetic bead.

31. The method of claim 29 or 30, wherein the cells are transfected by way of electroporation, Nucleofection, squeeze-poration, sonoporation, optical transfection, Magnetofection, and/or impalefection.

32. The method of any one of claims 6-18, wherein the cells are obtained by delivering to the cells a nuclease that catalyzes a single-strand break or a double-strand break at a target position within the genome of the cell, optionally wherein the target position is near or within a gene encoding an endogenous NOD2 protein.

33. The method of claim 32, wherein the nuclease is delivered to the cells in combination with a guide RNA (gRNA) that hybridizes to the target position within the genome of the cell.

34. The method of claim 32 or 33, wherein the nuclease is a clustered regularly interspaced short palindromic repeats (CRISPR)-associated protein.

35. The method of claim 34, wherein the CRISPR-associated protein is CRISPR-associated protein 9 (Cas9) or CRISPR-associated protein 12a (Cas12a).

36. The method of claim 32, wherein the nuclease is a transcription activator-like effector nuclease, a meganuclease, or a zinc finger nuclease.

37. The method of any one of claims 32-36, wherein the cells are additionally contacted with a template nucleic acid encoding NOD2 while the cells are contacted with the nuclease.

38. The method of claim 37, wherein the template nucleic acid molecule encoding NOD2 comprises a 5′ homology arm and a 3′ homology arm having nucleic acid sequences that are sufficiently similar to the nucleic acid sequences located 5′ to the target position and 3′ to the target position, respectively, to promote homologous recombination.

39. The method of claim 37 or 38, wherein the nuclease, gRNA, and template nucleic acid are delivered to the cells by contacting the cells with a viral vector that encodes the nuclease, gRNA, and template nucleic acid.

40. The method of claim 39, wherein the viral vector that encodes the nuclease, gRNA, and template nucleic acid is an adeno-associated virus (AAV), an adenovirus, a parvovirus, a coronavirus, a rhabdovirus, a paramyxovirus, a picornavirus, an alphavirus, a herpes virus, a poxvirus, or a Retroviridae family virus.

41. The method of claim 40, wherein the viral vector that encodes the nuclease, gRNA, and template nucleic acid is a Retroviridae family virus.

42. The method of claim 41, wherein the Retroviridae family virus is a lentiviral vector, alpharetroviral vector, or gammaretroviral vector.

43. The method of claim 41 or 42, wherein the Retroviridae family virus that encodes the nuclease, gRNA, and template nucleic acid comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

44. The method of claim 43, wherein the viral vector that encodes the nuclease, gRNA, and template nucleic acid is an integration-deficient lentiviral vector (IDLV).

45. The method of claim 44, wherein the viral vector that encodes the nuclease, gRNA, and template nucleic acid is an AAV selected from the group consisting of AAV1, AAV2, AAV3, AAV4, AAV5, AAV6, AAV7, AAV8, AAV9, AAV10, and AAVrh74, optionally wherein the AAV is AAV6.

46. The method of any one of claims 6-45, wherein prior to administering the composition to the patient, a population of precursor cells is isolated from the patient or a donor, and wherein the precursor cells are expanded ex vivo to yield the population of cells being administered to the patient.

47. The method of claim 46, wherein the precursor cells are CD34+ HSCs, and wherein the precursor cells are expanded without substantial loss of HSC functional potential.

48. The method of claim 46 or 47, wherein prior to isolation of the precursor cells from the patient or donor, the patient or donor is administered one or more pluripotent cell mobilization agents.

49. The method of any one of claims 6-48, wherein prior to administering the composition to the patient, a population of endogenous pluripotent cells is ablated in the patient by administration of one or more conditioning agents to the patient.

50. The method of any one of claims 6-48, the method comprising ablating a population of endogenous pluripotent cells in the patient by administering to the patient one or more conditioning agents prior to administering the composition to the patient.

51. The method of claim 49 or 50, wherein the one or more conditioning agents are non-myeloablative conditioning agents.

52. The method of any one of claims 6-51, wherein upon administration of the composition to the patient, the administered cells, or progeny thereof, differentiate into one or more cell types selected from megakaryocytes, thrombocytes, platelets, erythrocytes, mast cells, myeoblasts, basophils, neutrophils, eosinophils, microglia, granulocytes, monocytes, osteoclasts, antigen-presenting cells, macrophages, dendritic cells, natural killer cells, T-lymphocytes, and B-lymphocytes.

53. The method of any one of claims 5-52, wherein the nucleic acid molecule comprises a transgene encoding NOD2 operably linked to a ubiquitous promoter, optionally wherein the promoter is an EF1α promoter or an EFS promoter.

54. The method of any one of claims 5-52, wherein the nucleic acid molecule comprises a transgene encoding NOD2 operably linked to a tissue-specific promoter.

55. The method of claim 54, wherein the promoter is a CD11b promoter, sp146/p47 promoter, CD68 promoter, sp146/gp9 promoter, or an endogenous NOD2 promoter.

56. The method of any one of claims 1-55, wherein the patient is a mammal.

57. The method of claim 56, wherein the patient is a human.

58. The method of any one of claims 1-57, wherein the patient has a loss-of-function mutation in an endogenous gene encoding NOD2.

59. The method of claim 58, wherein the mutation is selected from the group consisting of R702W, G908R, and L1007fs.

60. The method of claim 58 or 59, wherein the mutation is heterozygous.

61. The method of claim 58 or 59, wherein the mutation is homozygous.

62. The method of any one of claims 1-61, wherein the patient has previously been treated with one or more immunosuppressive agents, biologic agents, and/or corticosteroids.

63. The method of claim 62, wherein the patient has not responded to treatment with the one or more immunosuppressive agents, biologic agents, and/or corticosteroids.

64. The method of claim 62 or 63, wherein the one or more immunosuppressive agents comprise azathioprine, methotrexate and/or infliximab.

65. The method of any one of claims 1-64, wherein, prior to providing the patient with the one or more agents that increase expression and/or activity of NOD2, the patient exhibits persistent disease activity, as assessed by endoscopy, colonoscopy, and/or magnetic resonance enterography.

66. The method of any one of claims 1-65, wherein, prior to providing the patient with the one or more agents that increase expression and/or activity of NOD2, the patient has been determined to be at risk of short bowel disease and/or refractory colonic disease if the patient were to undergo an imminent surgical procedure.

67. The method of any one of claims 1-66, wherein, prior to providing the patient with the one or more agents that increase expression and/or activity of NOD2, the patient exhibits a persistent perianal lesion such that the patient is not a candidate for coloproctectomy.

68. The method of any one of claims 1-67, wherein, prior to providing the patient with the one or more agents that increase expression and/or activity of NOD2, the patient exhibits impaired function and/or quality of life.

69. The method of claim 68, wherein function and/or quality of life are assessed by way of an Inflammatory Bowel Disease Questionnaire (IBDQ), a European Questionnaire of Life Quality, or a Karnofsky Index.

70. The method of any one of claims 1-69, wherein, after providing the patient with the one or more agents that increase expression and/or activity of NOD2, the patient exhibits sustained disease remission, optionally wherein the patient exhibits the sustained disease remission one year after providing the patient with the one or more agents that increase expression and/or activity of NOD2.

71. The method of any one of claims 1-70, wherein, after providing the patient with the one or more agents that increase expression and/or activity of NOD2, the patient no longer requires treatment with immunosuppressive agents, biologic agents, and/or corticosteroids, optionally wherein the patient does not require treatment with the immunosuppressive agents, biologic agents, and/or corticosteroids for at least three months.

72. The method of any one of claims 1-71, wherein, after providing the patient with the one or more agents that increase expression and/or activity of NOD2, the patient does not exhibit evidence of erosive disease in the gastrointestinal tract, as assessed by endoscopy and/or radiology.

73. A pharmaceutical composition comprising (i) a population of cells that express NOD2, optionally wherein the cells are pluripotent cells, and (ii) one or more carriers, diluents, and/or excipients.

74. The pharmaceutical composition of claim 73, wherein the cells are human cells.

75. The pharmaceutical composition of claim 73 or 74, wherein the cells are HSCs or HPCs.

76. The pharmaceutical composition of claim 73 or 74, wherein the cells are embryonic stem cells.

77. The pharmaceutical composition of claim 73 or 74, wherein the cells are induced pluripotent stem cells.

78. The pharmaceutical composition of claim 73 or 74, wherein the cells are CD34+ cells.

79. The pharmaceutical composition of claim 78, wherein the CD34+ cells are myeloid progenitor cells.

80. The pharmaceutical composition of any one of claims 73-79, wherein the composition is formulated for administration to a human patient.

81. The pharmaceutical composition of claim 80, wherein the composition is formulated for intravenous injection to the human patient.

82. The pharmaceutical composition of claim 80 or 81, wherein the cells are autologous with respect to the patient.

83. The pharmaceutical composition of claim 80 or 81, wherein the cells are allogeneic with respect to the patient.

84. The pharmaceutical composition of claim 83, wherein the cells are HLA-matched to the patient.

85. The pharmaceutical composition of any one of claims 73-84, wherein the cells comprise a transgene encoding NOD2 operably linked to a ubiquitous promoter, optionally wherein the promoter is an EF1α promoter or an EFS promoter.

86. The pharmaceutical composition of any one of claims 73-84, wherein the cells comprise a transgene encoding NOD2 operably linked to a tissue-specific promoter.

87. The pharmaceutical composition of claim 86, wherein the promoter is a CD11b promoter, sp146/p47 promoter, CD68 promoter, sp146/gp9 promoter, or an endogenous NOD2 promoter.

88. A pharmaceutical composition comprising (i) a viral vector that encodes NOD2 and (ii) one or more carriers, diluents, and/or excipients.

89. The pharmaceutical composition of claim 88, wherein the viral vector is selected from the group consisting of a Retroviridae family virus, an adeno-associated virus, an adenovirus, a parvovirus, a coronavirus, a rhabdovirus, a paramyxovirus, a picornavirus, an alphavirus, a herpes virus, and a poxvirus.

90. The pharmaceutical composition of claim 89, wherein the viral vector is a Retroviridae family viral vector.

91. The pharmaceutical composition of claim 90, wherein the Retroviridae family viral vector is a lentiviral vector.

92. The pharmaceutical composition of claim 90, wherein the Retroviridae family viral vector is an alpharetroviral vector or a gammaretroviral vector.

93. The pharmaceutical composition of any one of claims 90-92, wherein the Retroviridae family viral vector comprises a central polypurine tract, a woodchuck hepatitis virus post-transcriptional regulatory element, a 5′-LTR, HIV signal sequence, HIV Psi signal 5′-splice site, delta-GAG element, 3′-splice site, and a 3′-self inactivating LTR.

94. The pharmaceutical composition of any one of claims 90-93, wherein the viral vector is a pseudotyped viral vector.

95. The pharmaceutical composition of claim 94, wherein the pseudotyped viral vector selected from the group consisting of a pseudotyped adenovirus, a pseudotyped parvovirus, a pseudotyped coronavirus, a pseudotyped rhabdovirus, a pseudotyped paramyxovirus, a pseudotyped picornavirus, a pseudotyped alphavirus, a pseudotyped herpes virus, a pseudotyped poxvirus, and a pseudotyped Retroviridae family virus.

96. The pharmaceutical composition of claim 94 or 95, wherein the pseudotyped viral vector comprises one or more envelope proteins from a virus selected from vesicular stomatitis virus (VSV), RD114 virus, murine leukemia virus (MLV), feline leukemia virus (FeLV), Venezuelan equine encephalitis virus (VEE), human foamy virus (HFV), walleye dermal sarcoma virus (WDSV), Semliki Forest virus (SFV), Rabies virus, avian leukosis virus (ALV), bovine immunodeficiency virus (BIV), bovine leukemia virus (BLV), Epstein-Barr virus (EBV), Caprine arthritis encephalitis virus (CAEV), Sin Nombre virus (SNV), Cherry Twisted Leaf virus (ChTLV), Simian T-cell leukemia virus (STLV), Mason-Pfizer monkey virus (MPMV), squirrel monkey retrovirus (SMRV), Rous-associated virus (RAV), Fujinami sarcoma virus (FuSV), avian carcinoma virus (MH2), avian encephalomyelitis virus (AEV), Alfa mosaic virus (AMV), avian sarcoma virus CT10, and equine infectious anemia virus (EIAV).

97. The pharmaceutical composition of claim 96, wherein the pseudotyped viral vector comprises a VSV-G envelope protein.

98. The pharmaceutical composition of any one of claims 88-97, wherein the composition is formulated for administration to a human patient.

99. The pharmaceutical composition of claim 98, wherein the composition is formulated for intravenous injection to the human patient.

100. The pharmaceutical composition of any one of claims 88-99, wherein the viral vector comprises a transgene encoding NOD2 operably linked to a ubiquitous promoter, optionally wherein the promoter is an EF1α promoter or an EFS promoter.

101. The pharmaceutical composition of any one of claims 88-100, wherein the viral vector comprises a transgene encoding NOD2 operably linked to a tissue-specific promoter.

102. The pharmaceutical composition of claim 101, wherein the promoter is a CD11b promoter, sp146/p47 promoter, CD68 promoter, sp146/gp9 promoter, or an endogenous NOD2 promoter.

103. A kit comprising the pharmaceutical composition of any one of claims 73-102, wherein the kit further comprises a package insert instructing a user of the kit to administer the pharmaceutical composition to a human patient having Crohn's disease.

104. The kit of claim 103, wherein the package insert instructs a user of the kit to perform the method of any one of claims 1-72.


SEQUENCE LISTING (2 sequences)

SEQ ID NO: 1 - DNA, 4485 bp, Homo sapiens
gtagacagatccaggctcaccagtcctgtgccactgggcttttggcgttctgcacaaggcctacccgcagatgccatgcctgctcccccagcctaatgggctttgatgggggaagagggtggttcagcctctcacgatgaggaggaaagagcaagtgtcctcctcggacattctccgggttgtgaaatgtgctcgcaggaggcttttcaggcacagaggagccagctggtcgagctgctggtctcagggtccctggaaggcttcgagagtgtcctggactggctgctgtcctgggaggtcctctcctgggaggactacgagggcttccacctcctgggccagcctctctcccacttggccaggcgccttctggacaccgtctggaataagggtacttgggcctgtcagaagctcatcgcggctgcccaagaagcccaggccgacagccagtcccccaagctgcatggctgctgggacccccactcgctccacccagcccgagacctgcagagtcaccggccagccattgtcaggaggctccacagccatgtggagaacatgctggacctggcatgggagcggggtttcgtcagccagtatgaatgtgatgaaatcaggttgccgatcttcacaccgtcccagagggcaagaaggctgcttgatcttgccacggtgaaagcgaatggattggctgccttccttctacaacatgttcaggaattaccagtcccattggccctgcctttggaagctgccacatgcaagaagtatatggccaagctgaggaccacggtgtctgctcagtctcgcttcctcagtacctatgatggagcagagacgctctgcctggaggacatatacacagagaatgtcctggaggtctgggcagatgtgggcatggctggacccccgcagaagagcccagccaccctgggcctggaggagctcttcagcacccctggccacctcaatgacgatgcggacactgtgctggtggtgggtgaggcgggcagtggcaagagcacgctcctgcagcggctgcacttgctgtgggctgcagggcaagacttccaggaatttctctttgtcttcccattcagctgccggcagctgcagtgcatggccaaaccactctctgtgcggactctactctttgagcactgctgttggcctgatgttggtcaagaagacatcttccagttactccttgaccaccctgaccgtgtcctgttaacctttgatggctttgacgagttcaagttcaggttcacggatcgtgaacgccactgctccccgaccgaccccacctctgtccagaccctgctcttcaaccttctgcagggcaacctgctgaagaatgcccgcaaggtggtgaccagccgtccggccgctgtgtcggcgttcctcaggaagtacatccgcaccgagttcaacctcaagggcttctctgaacagggcatcgagctgtacctgaggaagcgccatcatgagcccggggtggcggaccgcctcatccgcctgctccaagagacctcagccctgcacggtttgtgccacctgcctgtcttctcatggatggtgtccaaatgccaccaggaactgttgctgcaggagggggggtccccaaagaccactacagatatgtacctgctgattctgcagcattttctgctgcatgccacccccccagactcagcttcccaaggtctgggacccagtcttcttcggggccgcctccccaccctcctgcacctgggcagactggctctgtggggcctgggcatgtgctgctacgtgttctcagcccagcagctccaggcagcacaggtcagccctgatgacatttctcttggcttcctggtgcgtgccaaaggtgtcgtgccagggagtacggcgcccctggaattccttcacatcactttccagtgcttctttgccgcgttctacctggcactcagtgctgatgtgccaccagctttgctcagacacctcttcaattgtggcaggccaggcaactcaccaatggccaggctcctgcccacgatgtgcatccaggcctcggagggaaaggacagcagcgtggcagctttgctgcagaaggccgagccgcacaaccttcagatcacagcagccttcctggcagggctgttgtcccgggagcactggggcctgctggctgagtgccagacatctgagaaggccctgctccggcgccaggcctgtgcccgctggtgtctggcccgcagcctccgcaagcacttccactccatcccgccagctgcaccgggtgaggccaagagcgtgcatgccatgcccgggttcatctggctcatccggagcctgtacgagatgcaggaggagcggctggctcggaaggctgcacgtggcctgaatgttgggcacctcaagttgacattttgcagtgtgggccccactgagtgtgctgccctggcctttgtgctgcagcacctccggcggcccgtggccctgcagctggactacaactctgtgggtgacattggcgtggagcagctgctgccttgccttggtgtctgcaaggctctgtatttgcgcgataacaatatctcagaccgaggcatctgcaagctcattgaatgtgctcttcactgcgagcaattgcagaagttagctctattcaacaacaaattgactgacggctgtgcacactccatggctaagctccttgcatgcaggcagaacttcttggcattgaggctggggaataactacatcactgccgcgggagcccaagtgctggccgaggggctccgaggcaacacctccttgcagttcctgggattctggggcaacagagtgggtgacgagggggcccaggccctggctgaagccttgggtgatcaccagagcttgaggtggctcagcctggtggggaacaacattggcagtgtgggtgcccaagccttggcactgatgctggcaaagaacgtcatgctagaagaactctgcctggaggagaaccatctccaggatgaaggtgtatgttctctcgcagaaggactgaagaaaaattcaagtttgaaaatcctgaagttgtccaataactgcatcacctacctaggggcagaagccctcctgcaggcccttgaaaggaatgacaccatcctggaagtctggctccgagggaacactttctctctagaggaggttgacaagctcggctgcagggacaccagactcttgctttgaagtctccgggaggatgttcgtctcagtttgtttgtgagcaggctgtgagtttgggccccagaggctgggtgacatgtgttggcagcctcttcaaaatgagccctgtcctgcctaaggctgaacttgttttctgggaacaccataggtcacctttattctggcagaggagggagcatcagtgccctccaggatagacttttcccaagcctacttttgccattgacttcttcccaagattcaatcccaggatgtacaaggacagcccctcctccatagtatgggactggcctctgctgatcctcccaggcttccgtgtgggtcagtggggcccatggatgtgcttgttaactgagtgccttttggtggagaggcccggcctctcacaaaagaccccttaccactgctctgatgaagaggagtacacagaacacataattcaggaagcagctttccccatgtctcgactcatccatccaggccattccccgtctctggttcctcccctcctcctggactcctgcacacgctccttcctctgaggctgaaattcagaatattagtgacctcagctttgatatttcacttacagcacccccaaccctggcacccagggtgggaagggctacaccttagcctgccctcctttccggtgtttaagacatttttggaaggggacacgtgacagccgtttgttccccaagacattctaggtttgcaagaaaaatatgaccacactccagctgggatcacatgtggacttttatttccagtgaaatcagttactcttcagttaagcctttggaaacagctcgactttaaaaagctccaaatgcagctttaaaaaattaatctgggccagaatttcaaacggcctcactaggcttctggttgatgcctgtgaactgaactctgacaacagacttctgaaatagacccacaagaggcagttccatttcatttgtgccagaatgctttaggatgtacagttatggattgaaagtttacaggaaaaaaaattaggccgttccttcaaagcaaatgtcttcctggattattcaaaatgatgtatgttgaagcctttgtaaattgtcagatgctgtgcaaatgttattattttaaacattatgatgtgtgaaaactggttaatatttataggtcactttgttttactgtcttaagtttatactcttatagacaacatggccgtgaactttatgctgtaaataatcagaggggaataaactgttgagtcaaaac

SEQ ID NO: 2 - protein, 1040 aa, Homo sapiens
MGEEGGSASHDEEERASVLLGHSPGCEMCSQEAFQAQRSQLVELLVSGSLEGFESVLDWLLSWEVLSWEDYEGFHLLGQPLSHLARRLLDTVWNKGTWACQKLIAAAQEAQADSQSPKLHGCWDPHSLHPARDLQSHRPAIVRRLHSHVENMLDLAWERGFVSQYECDEIRLPIFTPSQRARRLLDLATVKANGLAAFLLQHVQELPVPLALPLEAATCKKYMAKLRTTVSAQSRFLSTYDGAETLCLEDIYTENVLEVWADVGMAGPPQKSPATLGLEELFSTPGHLNDDADTVLVVGEAGSGKSTLLQRLHLLWAAGQDFQEFLFVFPFSCRQLQCMAKPLSVRTLLFEHCCWPDVGQEDIFQLLLDHPDRVLLTFDGFDEFKFRFTDRERHCSPTDPTSVQTLLFNLLQGNLLKNARKVVTSRPAAVSAFLRKYIRTEFNLKGFSEQGIELYLRKRHHEPGVADRLIRLLQETSALHGLCHLPVFSWMVSKCHQELLLQEGGSPKTTTDMYLLILQHFLLHATPPDSASQGLGPSLLRGRLPTLLHLGRLALWGLGMCCYVFSAQQLQAAQVSPDDISLGFLVRAKGVVPGSTAPLEFLHITFQCFFAAFYLALSADVPPALLRHLFNCGRPGNSPMARLLPTMCIQASEGKDSSVAALLQKAEPHNLQITAAFLAGLLSREHWGLLAECQTSEKALLRRQACARWCLARSLRKHFHSIPPAAPGEAKSVHAMPGFIWLIRSLYEMQEERLARKAARGLNVGHLKLTFCSVGPTECAALAFVLQHLRRPVALQLDYNSVGDIGVEQLLPCLGVCKALYLRDNNISDRGICKLIECALHCEQLQKLALFNNKLTDGCAHSMAKLLACRQNFLALRLGNNYITAAGAQVLAEGLRGNTSLQFLGFWGNRVGDEGAQALAEALGDHQSLRWLSLVGNNIGSVGAQALALMLAKNVMLEELCLEENHLQDEGVCSLAEGLKKNSSLKILKLSNNCITYLGAEALLQALERNDTILEVWLRGNTFSLEEVDKLGCRDTRLLL